(19) United States
(12) Patent Application Publication
(10) Pub. No.: US 2021/0145799 A1
(43) Pub. Date: May 20, 2021

(54) PHARMACEUTICAL COMBINATION PRODUCTS COMPRISING A HISTONE DEACETYLASE (HDAC) INHIBITOR AND A TLR7 AGONIST AND/OR TLR8 AGONIST FOR THE TREATMENT OF CANCER

(71) Applicant: 4SC AG, Planegg-Martinsried (DE)

(72) Inventor: Svetlana HAMM, Eichenau (DE)

(73) Assignee: 4SC AG, Planegg-Martinsried (DE)

(21) Appl. No.: 17/047,529

(22) PCT Filed: Apr. 15, 2019
(86) PCT No.: PCT/EP2019/059651

§ 371 Date: Oct. 14, 2020

(30) Foreign Application Priority Data
Apr. 14, 2018 (EP) 18167403.7

(51) Int. Cl. A61K 31/4155; A61K 31/4745; A61K 9/00; A61P 35/00

(52) U.S. Cl. CPC A61K 31/4155; A61P 35/00; A61K 9/0053; A61K 31/4745

(57) ABSTRACT

The invention relates to pharmaceutical combination products comprising an HD AC inhibitor, e.g. (E)-N-(2-amino-phenyl)-3-{1-[4-(1-methyl-1H-pyrazol-4-yl)-benzenesulfonyl]-1H-pyrrol-3-yl}-acrylamide as well as salts and or solvents thereof and TLR7 agonist and/or TLR8 agonist for the treatment of cancer. The present invention also relates to methods of treatment of patients suffering from cancer with these pharmaceutical combination products.

### TECHNICAL FIELD

The invention relates to pharmaceutical combination products comprising at least one HDAC inhibitor, e.g., (E)-N-(2-amino-phenyl)-3-{1-[4-(1-methyl-1H-pyrazol-4-yl)-benzenesulfonyl]-1H-pyrrol-3-yl}-acrylamide or a salt thereof and at least one TLR7 agonist and/or TLR8 agonist for the treatment of cancer. The present invention also relates to methods of treatment of patients suffering from cancer with these pharmaceutical combination products.

### TECHNICAL BACKGROUND

Cancer relates to a class of diseases in which cells have lost growth control, i.e. these cells divide beyond the normal limits. Cancer cells are often capable of invading adjacent tissues, thereby destroying the tissue integrity. Sometimes metastasis occurs, which is a process in which cancer cells spread to other locations in the body via lymph or blood. Cancer represents one of the leading causes of death in the world. Cancers can be classified according to the organ, tissue and cell-type from which the cancerous cells originate: brain, liver, bone, liver, kidney, skin, etc. While a number of anti-cancer drugs are available and significant progress has been made as to how to use these drugs, novel types of anti-cancer compounds and therapies are urgently needed.

Toll-Like Receptors (TLR) are cell-surface or endogenous receptors by which cells recognize Pathogen Associated Molecular Patterns (PAMP) (Killen SD 2006). These receptors are involved in the recognition of molecular patterns, e.g., single-stranded and double-stranded RNA, double-stranded DNA, LPS, lipoteichoic acid, etc. TLR's are mainly expressed by cells of the immune system. Immunotherapeutic treatments based on the use of TLR9 ligands had been tested for the treatment of solid cancers, such as NSCLC (Kanzler H. et al. 2007 Nature Medicine, 13:532; Krieg A. M. 2007, JCI, 117:1184).

US patent application 11,184,191 to Lebecque et al. describes methods for treating Toll-like receptor expressing cancers and tumor cells by selecting a TLR expressing tumor cell and contacting the cell with a therapeutically effective amount of a TLR ligand. In particular, 11,184,191 describes methods for treating TLR3 expressing cancers and tumors cells using TLR3 agonists. The TLR3 agonist induces apoptosis of the tumor cells expressing TLR3.

WO 2017/181128 discloses a method of treating cancer in a mammal, the method comprising administering to the subject an effective amount of an immunogenic composition by intratumoural delivery of an immunogenic composition comprising a particle comprising a TLR nine agonist and tumour antigen associated with the biocompatible modularisation agent.

The use of TLR7 or 8 agonists as adjuvants to prime the antitumoral immune response is also known from various publications. The lead compound of the imidazoquinoline family, imiquimod, is marketed as a topical formulation for use against primary skin tumors and cutaneous metastasis (Schón & Schón, Oncogene, 2008). In skin cancer, increased immune functions, particularly enhancement of Natural Killer cell was observed and antitumor activity, dendritic cell maturation and T cell immunity to tumor antigens (Schon 2008, Kanzler 2007, Stary 2007) was described.

There is a persistent need for new therapies of different types of cancers using anti-cancer drugs, alongside with agents stimulating the affected body's own immune system. The present invention addresses this need.

### SUBJECT MATTER OF THE INVENTION

Subject matter of the invention according to a first item is a pharmaceutical combination product comprising a compound of Formula (I)

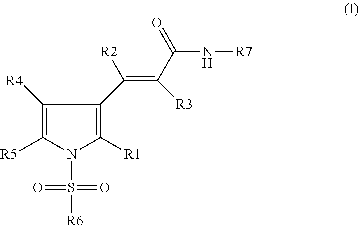

in which
R1, R4 and R5 are independently hydrogen, 1-4C-alkyl, halogen, 1-4C-alkoxy,
R2 and R3 are independently hydrogen or 1-4C-alkyl,
R6 is -T1Q1, in which T1 is a bond or 1-4C-alkylene,
either Q1 is substituted by R61 and/or R62, and is Aa1, Hh1, Ha1, Ha2, Ha3, Ha4 or Ah1, or
Q1 is unsubstituted, and is Ha2, Ha3 or Ha4, in which
R61 is 1-4C-alkyl, phenyl-1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, hydroxy-1-4C-alkyl, 1-4C-alkoxy-1-4C-alkyl, 1-4C-alkylsulphonylamino, tolylsulphonylamino, phenylsulphonylamino, 1-4C-alkylcarbonylamino, carbamoyl, sulphamoyl, mono- or di-1-4C-alkylaminocarbonyl, mono- or di-1-4C-alkylaminosulphonyl, -T2-N(R611)R612, —U-T3-N(R613)R614, -T4-Het3, or —V-T5-Het4, in which
T2 is a bond or 1-4C-alkylene,
R611 is hydrogen, 1-4C-alkyl, 3-7C-cycloalkyl, 3-7C-cycloalkylmethyl, hydroxy-2-4C-alkyl, 1-4C-alkoxy-2-4C-alkyl, 1-4C-alkylcarbonyl, or 1-4C-alkylsulphonyl,
R612 is hydrogen or 1-4C-alkyl,
or R611 and R612 together and with inclusion of the nitrogen atom, to which they are bonded, form a heterocyclic ring Het1, in which Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl, 3-7C-cycloalkylmethyl, hydroxy-2-4C-alkyl or 1-4C-alkoxy-2-4C-alkyl, 1-4C-alkylcarbonyl, or 1-4C-alkylsulphonyl
R614 is hydrogen or 1-4C-alkyl,
or R613 and R614 together and with inclusion of the nitrogen atom, to which they are bonded, form a heterocyclic ring Het2, in which
Het2 is morpholino, thiomorpholino, S-oxo-thiomorpholino, S,S-dioxo-thiomorpholino, piperidino, pyrrolidino, piperazino, or 4N-(1-4C-alkyl)-piperazino,
T4 is a bond or 1-4C-alkylene,
Het3 is 1N-(1-4C-alkyl)-piperidinyl or 1N-(1-4C-alkyl)-pyrrolidinyl,
V is —O— (oxygen) or —C(O)NH—,
T5 is a bond or 1-4C-alkylene,
Het4 is 1N-(1-4C-alkyl)-piperidinyl or 1N-(1-4C-alkyl)-pyrrolidinyl,
R62 is 1-4C-alkyl, 1-4C-alkoxy or halogen,
Aa1 is a bisaryl radical made up of two aryl groups,
which are selected independently from a group consisting of phenyl and naphthyl,
and
which are linked together via a single bond,
Hh1 is a bisheteroaryl radical made up of two heteroaryl groups,
which are selected independently from a group consisting of monocyclic 5- or 6-membered heteroaryl radicals comprising one or two heteroatoms, each of which is selected from the group consisting of nitrogen, oxygen and sulfur, and
which are linked together via a single bond,
Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
Ha4 is a heteroarylaryl radical made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha4 is bonded via said aryl moiety to the to the parent molecular group,
R7 is hydroxyl, or Cycl, in which Cycl is a ring system of formula Ia

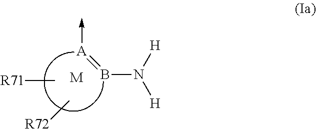

in which
A and B are C (carbon),
R71 and R72 are independently hydrogen, halogen, 1-4C-alkyl, or 1-4C-alkoxy,
M with inclusion of A and B is either a ring Ar2 or a ring Har2, in which Ar2 is a benzene ring, Har2 is a monocyclic 5- or 6-membered unsaturated heteroaromatic ring comprising one to three heteroatoms, each of which is selected from the group consisting of nitrogen, oxygen and sulfur,
and/or a salt or solvate of these compounds,
further comprising a TLR7 and/or TLR8 agonist for use in the treatment of cancer.

In a second item, subject matter of the present invention is also pharmaceutical combination product comprising a compound of Formula (I) and a TLR7 and/or TLR8 agonist for use in the treatment of cancer according to item 1,

wherein in the compound of formula (I),
R1, R4 and R5 are independently hydrogen, 1-4C-alkyl, halogen, or 1-4C-alkoxy,
R2 and R3 are independently hydrogen or 1-4C-alkyl,
R6 is -T1-Q1, in which T1 is a bond or 1-4C-alkylene,
either
Q1 is substituted by R61 and/or R62, and is Aa1, Hh1, Ha1, Ha2, Ha3 or Ah1,
or Q1 is unsubstituted, and is Ha2 or Ha3,
in which
R61 is 1-4C-alkyl, phenyl-1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, hydroxy-1-4C-alkyl, 1-4C-alkoxy-1-4C-alkyl, 1-4C-alkylsulphonylamino, tolylsulphonylamino, phenylsulphonylamino, 1-4C-alkylcarbonylamino, carbamoyl, sulphamoyl, mono- or di-1-4C-alkylaminocarbonyl, mono- or di-1-4C-alkylaminosulphonyl, -T2-N(R611)R612, or —U-T3-N(R613)R614, in which
T2 is a bond or 1-4C-alkylene,
R611 is hydrogen, 1-4C-alkyl, 3-7C-cycloalkyl, 3-7C-cycloalkylmethyl, hydroxy-2-4C-alkyl or 1-4C-alkoxy-2-4C-alkyl,
R612 is hydrogen or 1-4C-alkyl,
or R611 and R612 together and with inclusion of the nitrogen atom, to which they are bonded, form a heterocyclic ring Het1, in which
Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl, 3-7C-cycloalkylmethyl, hydroxy-2-4C-alkyl or 1-4C-alkoxy-2-4C-alkyl,
R614 is hydrogen or 1-4C-alkyl,
or R613 and R614 together and with inclusion of the nitrogen atom, to which they are bonded, form a heterocyclic ring Het2, in which Het2 is morpholino, thiomorpholino, S-oxo-thiomorpholino, S,S-dioxo-thiomorpholino, piperidino, pyrrolidino, piperazino, or 4N-(1-4C-alkyl)-piperazino,
R62 is 1-4C-alkyl, 1-4C-alkoxy or halogen,
Aa1 is a bisaryl radical made up of two aryl groups,
   which are selected independently from a group consisting of phenyl and naphthyl, and
   which are linked together via a single bond,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
R7 is hydroxyl, or Cycl, in which Cycl is a ring system of formula Ia

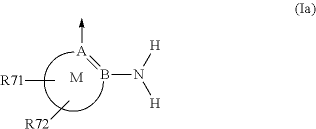

in which
A and B are C (carbon),
R71 and R72 are independently hydrogen, halogen, 1-4C-alkyl, or 1-4C-alkoxy,
M with inclusion of A and B is either a ring Ar2 or a ring Har2, in which
Ar2 is a benzene ring,
Har2 is a monocyclic 5- or 6-membered unsaturated heteroaromatic ring comprising one to three heteroatoms, each of which is selected from the group consisting of nitrogen, oxygen and sulfur,
and/or the salts or solvates or of these compounds
and
a TLR7 and/or TLR8 agonist.

In a third item, subject matter of the present invention is the pharmaceutical combination product comprising a compound of Formula (I) and a TLR7 and/or TLR8 agonist for use in the treatment of cancer according to item 1 or item 2,
wherein in the compound of formula (I),
R1, R4 and R5 are independently hydrogen, 1-4C-alkyl, halogen, or 1-4C-alkoxy,
R2 and R3 are independently hydrogen or 1-4C-alkyl,
R6 is -T1-Q1, in which T1 is a bond, or 1-4C-alkylene,
either
Q1 is substituted by R61 and/or R62, and is Aa1, Hh1, Ha1, Ha2, Ha3 or Ah1,
or Q1 is unsubstituted, and is Ha2 or Ha3,
in which
R61 is 1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, or -T2-N(R611)R612, in which
T2 is a bond or 1-4C-alkylene,
R611 and R612 are indenpendently hydrogen or 1-4C-alkyl,
or
R611 and R612 together and with inclusion of the nitrogen atom, to which they are bonded, form a heterocyclic ring Het1, in which
Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
R62 is 1-4C-alkyl, 1-4C-alkoxy or halogen,
Aa1 is a bisaryl radical made up of two aryl groups,
   which are selected independently from a group consisting of phenyl and naphthyl, and which are linked together via a single bond,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an aryl-heteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
R7 is hydroxyl, or Cycl, in which Cycl is a ring system of formula Ia

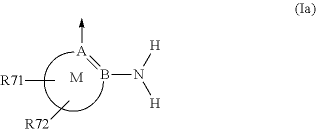

in which
A and B are C (carbon),
R71 and R72 are independently hydrogen, halogen, 1-4C-alkyl, or 1-4C-alkoxy,
M with inclusion of A and B is either a ring Ar2 or a ring Har2, in which Ar2 is a benzene ring,
Har2 is a monocyclic 5- or 6-membered unsaturated heteroaromatic ring comprising one to three heteroatoms, each of which is selected from the group consisting of nitrogen, oxygen and sulfur,
and/or a salt or a solvate of these compounds.

According to item 4, subject matter of the present invention is a pharmaceutical combination product comprising a compound of Formula (I) and a TLR7 and/or TLR8 agonist for use in the treatment of cancer according to any one of items 1 to 3, wherein in the compound of formula (I) is (E)-N-(2-amino-phenyl)-3-{1-[4-(1-methyl-1H-pyrazol-4-yl)-benzenesulfonyl]-1H-pyrrol-3-yl}-acrylamide or a salt or solvate thereof.

According to item 5, subject matter of the present invention is the pharmaceutical combination product comprising a compound of Formula (I) and a TLR7 and/or TLR8 agonist for use in the treatment of cancer according to any one of items 1 to 4, wherein in the compound of formula (I) is (E)-N-(2-amino-phenyl)-3-{1-[4-(1-methyl-1H-pyrazol-4-yl)-benzenesulfonyl]-1H-pyrrol-3-yl}-acrylamide or salt thereof, wherein the salt is the tosylate salt.

According to item 6, subject matter of the present invention is the pharmaceutical combination product comprising a compound of Formula (I) and a TLR7 and/or TLR8 agonist for use in the treatment of cancer according to any one of items 1 to 5, wherein the TLR7 and/or TLR8 agonist is a TLR8 agonist selected from the group comprising bazlitoran sodium, motolimod, NKTR-262, resiquimod, RSLV-132, durvalumab+MEDI-9197, E-6742, GS-9688, IMO-9200, MEDI-9197, VTX-1463, DN-1508052, 854-A, DV-1001, JB-6121, SC-2, CPG-52364, IMO-4200, VTX-294, VTX-763, IPH-3201, and CUCPT-8m, Gardiquimod, Imiquimod and R848 (resiquimod); or a TLR7 agonist selected from the group comprising Gardiquimod, Imiquimod and R848 (resiquimod), hydroxychloroquine bazlitoran sodium, GSK-2245035, NKTR-262, RSLV-132, vesatolimod, 852, AL-034, durvalumab+MEDI-9197, E-6742, IMO-9200, MEDI-9197, PAL-TIV, RG-7854, 854-A, AT-791, DSP-0509, DSR-6434, DV-1001, E-6446, GS-986, imiquimod SR, JB-6121, MBS-2, MBS-5, S-34240, SC-1, SC-2, DV-1079, 852-A, AZ-12441970, CPG-52364, DV-1179, imiquimod, IMO-3100, IMO-4200, IRS-661, isatoribine, loxoribine, bazlitoran sodium, GSK-2245035, NKTR-262, RSLV-132, vesatolimod, 852, AL-034, durvalumab+MEDI-9197, E-6742, IMO-9200, MEDI-9197, PAL-TIV, RG-7854, 854-A, AT-791, DSP-0509, DSR-6434, DV-1001, E-6446, GS-986, imiquimod SR, JB-6121, MBS-2, MBS-5, S-34240, SC-1, SC-2, DV-1079, 852-A, AZ-12441970, CPG-52364, DV-1179, imiquimod, IMO-3100, LMO-4200, IRS-661, isatoribine, loxoribine, SB-9922, PF-4878691, RG-7795, SM-324405, SM-276001, sotirimod, TMX-202, TMX-201, TMX-302, ANA-971, ANA-975, DSP-3025, IPH-3201, RG-7863, HUM-8P.342. Particularly, the TLR7 and/or TLR8 agonist is selected from Gardiquimod, Imiquimod and R848 (resiquimod).

According to item 7, subject matter of the present invention is the pharmaceutical combination product comprising a compound of Formula (I) and a TLR7 and/or TLR8 agonist for use in the treatment of cancer according to any one of items 1 to 6, wherein the TLR7 and/or TLR8 agonist is resiquimod.

According to item 8, subject matter of the present invention is pharmaceutical combination product comprising a compound of Formula (I) and a TLR7 and/or TLR8 agonist for use in the treatment of cancer according to any one of items 1 to 7, wherein the compound of formula (I) and the TLR7 and/or TLR8 agonist are administered concomitantly or separately.

According to item 9, subject matter of the present invention is the pharmaceutical combination product comprising a compound of Formula (I) and a TLR7 and/or TLR8 agonist for use in the treatment of cancer according to any one of items 1 to 8, wherein the compound of formula (I) and the TLR7 and/or TLR8 agonist are administered separately.

According to item 10, subject matter of the present invention is the pharmaceutical combination product comprising a compound of Formula (I) and a TLR7 and/or TLR8 agonist for use in the treatment of cancer according to any one of items 1 to 9, wherein the compound of Formula (I) is administered initially and the TLR7 and/or TLR8 agonist is administered subsequently.

According to item 11, subject matter of the present invention is the pharmaceutical combination product comprising a compound of Formula (I) and a TLR7 and/or TLR8 agonist for use in the treatment of cancer according to any one of items 1 to 10, wherein the compound of Formula (I) is formulated for oral administration and the TLR7 and/or TLR8 agonist is formulated for oral, parenteral or enteral administration, particularly for oral adminstration.

According to item 12, subject matter of the present invention is the pharmaceutical combination product comprising a compound of Formula (I) and a TLR7 and/or TLR8 agonist for use in the treatment of cancer according to any one of items 1 to 11, wherein the cancer is selected from the group comprising hepatocarcinoma, adrenocortical carcinoma, AIDS-related cancers including AIDS-related lymphoma, anal cancer, basal cell carcinoma, bile duct cancer, bone cancer, brain tumors including brain stem glioma, cerebellar astrocytoma, cerebral astrocytoma, malignant glioma, ependymoma, medulloblastoma, supratentorial primitive neuroectodermal tumors, visual pathway and hypothalamic glioma, breast cancer, bronchial adenomas/carcinoids, Burkitt's lymphoma, gastrointestinal, carcinoma of unknown primary site, central nervous system lymphoma, cervical cancer, chronic myeloproliferative disorders, colon cancer, colorectal cancer, cutaneous T-cell lymphoma, endometrial cancer, ependymoma, esophageal cancer, extracranial germ cell tumor, extragonadal germ cell tumor, ovarian germ cell tumor, eye cancer including intraocular melanoma and retinoblastoma, gallbladder cancer, gastrointestinal carcinoid tumor, gestational trophoblastic tumor, glioma, childhood brain stem glioma, head and neck cancer, hematologic cancer, adult and childhood (primary) hepatocellular cancer, hypopharyngeal cancer, islet cell or pancreatic cancer, renal cancer, laryngeal cancer, acute lymphoblastic leukemia, adult and childhood acute myeloid leukemia, chronic lymphocytic leukemia, chronic myelogenous leukemia, hairy cell leukemia, lip and oral cavity cancer, liver cancer, lung cancer, including non-small cell lung cancer and small cell lung cancer, Hodgkin's lymphoma, non-Hodgkin's lymphoma, primary central nervous system lymphoma, Waldenstrom's macroglobulinemia, merkel cell carcinoma, mesothelioma, metastatic squamous neck cancer with occult primary site, multiple endocrine neoplasia syndrome, multiple myeloma/plasma cell neoplasm, mycosis fungoides, myelodysplastic syndromes, myelodysplastic myeloproliferative diseases, multiple myeloma, chronic myeloproliferative disorders, nasal cavity and paranasal sinus cancer, nasopharyngeal cancer, neuroblastoma, oral cancer, oropharyngeal cancer, osteosarcoma/malignant fibrous histiocytoma of bone, ovarian cancer, ovarian epithelial cancer, ovarian low malignant potential tumor, pancreatic cancer, parathyroid cancer, penile cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter cancer, transitional cell cancer, rhabdomyosarcoma, salivary gland cancer, Ewing's sarcoma, Kaposi's sarcoma, soft tissue sarcoma, uterine sarcoma, sezary syndrome, skin cancer, including melanoma and non-melanoma skin cancer, small intestine cancer, squamous cell carcinoma, gastric cancer, supratentorial primitive neuroectodermal tumors, testicular cancer, thymoma, thymoma and thymic carcinoma, thyroid cancer, trophoblastic tumor, gestational, endometrial uterine cancer, uterine sarcoma, vaginal cancer, vulvar cancer, Waldenstrom's macroglobulinemia, Wilms' tumor.

According to item 13, subject matter of the present invention is the pharmaceutical combination product comprising a compound of Formula (I) and a TLR7 and/or TLR8 agonist for use in the treatment of cancer according to any one of items 1 to 12, wherein the cancer is selected from the group comprising cancer of the prostate, bladder, kidney, muscle, ovary, skin, lung, pancreas, breast, cervix, colon, liver, connective tissue, placenta, bone, brain, uterus, salivary gland, or testes.

### FIGURE DESCRIPTION

FIG. 1. C38 model, mean values over time, days on the x-axes: days, tumor volume(mm3) on the y-axes.

FIG. 2: Tumor curves for individual animals, days on the x-axes: days, tumor volume(mm3) on the y-axes. A: vehicle, B. R848, C. 4SC-202, D. 4SC-202+R848

### DEFINITIONS AND EMBODIMENTS OF THE INVENTION

as used herein, the pharmaceutical combination products may comprise more than one pharmaceutical formulation, or the active compounds, i.e. the HDAC inhibitors, and the TFR7 and/or TFR8 agonists may be present in one pharmaceutical formulation, i.e. a pharmaceutical formulation comprising more than one active ingredient of formula (I) and the TFR7 and/or TFR8 agonists, or the respective active agents may be administered in separate pharmaceutical formulations.

In one embodiment according to the present invention, a pharmaceutical formulation comprises the pharmaceutical combination product according to the present invention, i.e. the active ingredients are present in the same pharmaceutical formulation. In other embodiments of the present invention, the combination products comprise at least one pharmaceutical formulation comprising an HD AC inhibitor and at least one further pharmaceutical formulation at least one TLR7 or TLR8 agonist. In those embodiments, where more than one pharmaceutical formulation according to the present invention is administered to a patient in need thereof, the order of administration of the respective formulations is not relevant, as long as the pharmaceutical formulations are administered at essentially the same point in time, for example, simultaneously, or within a period of about 1 to several hours, e.g., within an interval of 12 hours, 9 hours, 6 hours, 3 hours, 60 minutes, 45 minutes, 30 minutes, 15 minutes, 10 minutes, 5 minutes, or shorter.

### HDAC Inhibitors

The HDAC inhibitors of formula (I) in the pharmaceutical combination products of the present invention will be described below.

Formula (I) is

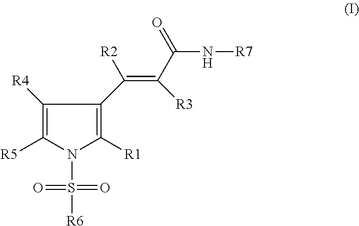

in which
R1, R4 and R5 are independently hydrogen, 1-4C-alkyl, halogen, or 1-4C-alkoxy,
R2 and R3 are independently hydrogen or 1-4C-alkyl,
R6 is -T1-Q1, in which T1 is a bond or 1-4C-alkylene,
either
Q1 is substituted by R61 and/or R62, and is Aa1, Hh1, Ha1, Ha2, Ha3, Ha4 or Ah1,
or
Q1 is unsubstituted, and is Ha2, Ha3 or Ha4,
in which
R61 is 1-4C-alkyl, phenyl-1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, hydroxy-1-4C-alkyl, 1-4C-alkoxy-1-4C-alkyl, 1-4C-alkylsulphonylamino, tolylsulphonylamino, phenylsulphonylamino, 1-4C-alkylcarbonylamino, carbamoyl, sulphamoyl, mono- or di-1-4C-alkylaminocarbonyl, mono- or di-1-4C-alkylaminosulphonyl, -T2-N(R611)R612, —U-T3-N(R613)R614, -T4-Het3, or —V-T5-Het4, in which
T2 is a bond or 1-4C-alkylene,
R611 is hydrogen, 1-4C-alkyl, 3-7C-cycloalkyl, 3-7C-cycloalkylmethyl, hydroxy-2-4C-alkyl, 1-4C-alkoxy-2-4C-alkyl, 1-4C-alkylcarbonyl, or 1-4C-alkylsulphonyl,
R612 is hydrogen or 1-4C-alkyl,
or R611 and R612 together and with inclusion of the nitrogen atom, to which they are bonded, form a heterocyclic ring Het1, in which Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl, 3-7C-cycloalkylmethyl, hydroxy-2-4C-alkyl or 1-4C-alkoxy-2-4C-alkyl, 1-4C-alkylcarbonyl, or 1-4C-alkylsulphonyl
R614 is hydrogen or 1-4C-alkyl,
or R613 and R614 together and with inclusion of the nitrogen atom, to which they are bonded, form a heterocyclic ring Het2, in which
Het2 is morpholino, thiomorpholino, S-oxo-thiomorpholino, S,S-dioxo-thiomorpholino, piperidino, pyrrolidino, piperazino, or 4N-(1-4C-alkyl)-piperazino,
T4 is a bond or 1-4C-alkylene,
Het3 is 1N-(1-4C-alkyl)-piperidinyl or 1N-(1-4C-alkyl)-pyrrolidinyl,
V is —O— (oxygen) or —C(O)NH—,
T5 is a bond or 1-4C-alkylene,
Het4 is 1N-(1-4C-alkyl)-piperidinyl or 1N-(1-4C-alkyl)-pyrrolidinyl,
R62 is 1-4C-alkyl, 1-4C-alkoxy or halogen,
Aa1 is a bisaryl radical made up of two aryl groups,
   which are selected independently from a group consisting of phenyl and naphthyl, and
   which are linked together via a single bond,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an arylheteroaryl radical made up of an aryl group selected from a g you you group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
Ha4 is a heteroarylaryl radical made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha4 is bonded via said aryl moiety to the to the parent molecular group,
R7 is hydroxyl, or Cycl, in which Cycl is a ring system of formula Ia

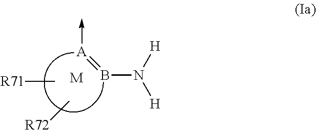

in which
A and B are C (carbon),
R71 and R72 are independently hydrogen, halogen, 1-4C-alkyl, or 1-4C-alkoxy,
M with inclusion of A and B is either a ring Ar2 or a ring Har2, in which Ar2 is a benzene ring, Har2 is a monocyclic 5- or 6-membered unsaturated heteroaromatic ring comprising one to three heteroatoms, each of which is selected from the group consisting of nitrogen, oxygen and sulfur,
and the salts of these compounds.

The HDAC inhibitor is in a second aspect (aspect B), which is an embodiment of aspect A, a compound of formula I,
in which
R1, R4 and R5 are independently hydrogen, 1-4C-alkyl, halogen, or 1-4C-alkoxy,
R2 and R3 are independently hydrogen or 1-4C-alkyl,
R6 is -T1-Q1, in which T1 is a bond or 1-4C-alkylene,
either
Q1 is substituted by R61 and/or R62, and is Aa1, Hh1, Ha1, Ha2, Ha3 or Ah1, or Q1 is unsubstituted, and is Ha2 or Ha3,
in which
R61 is 1-4C-alkyl, phenyl-1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, hydroxy-1-4C-alkyl, 1-4C-alkoxy-1-4C-alkyl, 1-4C-alkylsulphonylamino, tolylsulphonylamino, phenylsulphonylamino, 1-4C-alkylcarbonylamino, carbamoyl, sulphamoyl, mono- or di-1-4C-alkylaminocarbonyl, mono- or di-1-4C-alkylaminosulphonyl, -T2-N(R611)R612, or —U-T3-N(R613)R614, in which
T2 is a bond or 1-4C-alkylene,
R611 is hydrogen, 1-4C-alkyl, 3-7C-cycloalkyl, 3-7C-cycloalkylmethyl, hydroxy-2-4C-alkyl or 1-4C-alkoxy-2-4C-alkyl,
R612 is hydrogen or 1-4C-alkyl,
or R611 and R612 together and with inclusion of the nitrogen atom, to which they are bonded, form a heterocyclic ring Het1, in which
Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl, 3-7C-cycloalkylmethyl, hydroxy-2-4C-alkyl or 1-4C-alkoxy-2-4C-alkyl,
R614 is hydrogen or 1-4C-alkyl,
or R613 and R614 together and with inclusion of the nitrogen atom, to which they are bonded, form a heterocyclic ring Het2, in which Het2 is morpholino, thiomorpholino, S-oxo-thiomorpholino, S,S-dioxo-thiomorpholino, piperidino, pyrrolidino, piperazino, or 4N-(1-4C-alkyl)-piperazino,
R62 is 1-4C-alkyl, 1-4C-alkoxy or halogen,
Aa1 is a bisaryl radical made up of two aryl groups,
   which are selected independently from a group consisting of phenyl and naphthyl, and which are linked together via a single bond,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
R7 is hydroxyl, or Cycl, in which Cycl is a ring system of formula Ia

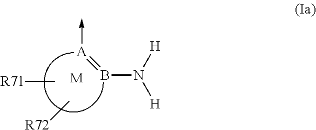

in which
A and B are C (carbon),
R71 and R72 are independently hydrogen, halogen, 1-4C-alkyl, or 1-4C-alkoxy,
M with inclusion of A and B is either a ring Ar2 or a ring Har2, in which
Ar2 is a benzene ring,
Har2 is a monocyclic 5- or 6-membered unsaturated heteroaromatic ring comprising one to three heteroatoms, each of which is selected from the group consisting of nitrogen, oxygen and sulfur,
and the salts of these compounds.

The HDAC inhibitor is in a third aspect (aspect C), which is also an embodiment of aspect A, a compound of formula I,
in which
R1, R4 and R5 are independently hydrogen, 1-4C-alkyl, halogen, or 1-4C-alkoxy,
R2 and R3 are independently hydrogen or 1-4C-alkyl,
R6 is -T1-Q1, in which T1 is a bond, or 1-4C-alkylene, either
Q1 is substituted by R61 and/or R62, and is Aa1, Hh1, Ha1, Ha2, Ha3 or Ah1, or Q1 is unsubstituted, and is Ha2 or Ha3,
in which
R61 is 1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, or -T2-N(R611)R612, in which
T2 is a bond or 1-4C-alkylene,
R611 and R612 are independently hydrogen or 1-4C-alkyl,
or R611 and R612 together and with inclusion of the nitrogen atom, to which they are bonded, form a heterocyclic ring Het1, in which
Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
R62 is 1-4C-alkyl, 1-4C-alkoxy or halogen,
Aa1 is a bisaryl radical made up of two aryl groups, which are selected independently from a group consisting of phenyl and naphthyl, and which are linked together via a single bond,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an aryl-heteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
   R7 is hydroxyl, or Cycl, in which Cycl is a ring system of formula Ia

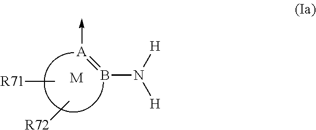

in which
A and B are C (carbon),
R71 and R72 are independently hydrogen, halogen, 1-4C-alkyl, or 1-4C-alkoxy,
M with inclusion of A and B is either a ring Ar2 or a ring Har2, in which Ar2 is a benzene ring,
Har2 is a monocyclic 5- or 6-membered unsaturated heteroaromatic ring comprising one to three heteroatoms, each of which is selected from the group consisting of nitrogen, oxygen and sulfur,
and the salts of these compounds.

As used herein, 1-4C-Alkyl represents a straight-chain or branched alkyl radical having 1 to 4 carbon atoms. Examples which may be mentioned are the butyl, isobutyl, sec-butyl, tert-butyl, propyl, isopropyl and particularly the ethyl and methyl radicals.

As used herein, 2-4C-Alkyl represents a straight-chain or branched alkyl radical having 2 to 4 carbon atoms. Examples which may be mentioned are the butyl, isobutyl, sec-butyl, tert-butyl, propyl, isopropyl and particularly the ethyl radicals.

As used herein, 3-7C-Cycloalkyl stands for cyclopropyl, cyclobutyl, cyclopentyl, cyclohexyl and cycloheptyl, of which cyclopropyl, cyclobutyl and cyclopentyl are particular examples.

As used herein, 3-7C-Cycloalkylmethyl stands for a methyl radical, which is substituted by one of the abovementioned 3-7C-cycloalkyl radicals. Particular examples which may be mentioned are the cyclopropylmethyl, the cyclobutylmethyl and the cyclopentylmethyl radicals.

As used herein, 1-4C-Alkylene is a branched or, particularly, straight chain alkylene radical having 1 to 4 carbon atoms. Examples which may be mentioned are the methylene (—CH₂—), ethylene (dimethylene) (—CH₂—CH₂—), trimethylene (—CH₂—CH₂—CH₂—) and the tetramethylene (—CH₂—CH₂—CH₂—CH₂—) radical.

As used herein, 2-4C-Alkylene is a branched or, particularly, straight chain alkylene radical having 2 to 4 carbon atoms. Examples which may be mentioned are the ethylene (dimethylene) (—CH₂—CH₂—), trimethylene (—CH₂—CH₂—CH₂—) and the tetramethylene (—CH₂—CH₂—CH₂—CH₂—) radical.

As used herein, 1-4C-Alkoxy represents radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy and particularly the ethoxy and methoxy radicals.

As used herein, 1-4C-Alkoxy-1-4C-alkyl stands for one of the abovementioned 1-4C-alkyl radicals, which is substituted by one of the abovementioned 1-4C-alkoxy radicals. Examples which may be mentioned are the methoxymethyl, 2-methoxyethyl, 3-methoxypropyl and the 2-ethoxyethyl radical.

As used herein, 1-4C-Alkoxy-2-4C-alkyl stands for one of the abovementioned 2-4C-alkyl radicals, which is substituted by one of the abovementioned 1-4C-alkoxy radicals. Examples which may be mentioned are the 2-methoxyethyl, 3-methoxypropyl and the 2-ethoxyethyl radical.

As used herein, Hydroxy-1-4C-alkyl stands for one of the abovementioned 1-4C-alkyl radicals which is substituted by hydroxyl. Examples which may be mentioned are the hydroxymethyl radical, the 2-hydroxyethyl radical or the 3-hydroxypropyl radical.

As used herein, Hydroxy-2-4C-alkyl stands for one of the abovementioned 2-4C-alkyl radicals which is substituted by hydroxyl. Examples which may be mentioned are the 2-hydroxyethyl radical or the 3-hydroxypropyl radical.

As used herein, Phenyl-1-4C-alkyl represents one of the abovementioned 1-4C-alkyl radicals, which is substituted by a phenyl radical. Examples which may be mentioned are the benzyl and phenethyl radicals.

As used herein, Mono- or Di-1-4C-alkylamino radicals contain in addition to the nitrogen atom, one or two of the abovementioned 1-4C-alkyl radicals. Particular examples red are the di-1-4C-alkylamino radicals, especially the dimethylamino, the diethylamino and the diisopropylamino radical.

As used herein, Mono- or Di-1-4C-alkylaminocarbonyl radicals contain in addition to the carbonyl group one of the abovementioned mono- or di-1-4C-alkylamino radicals. Examples which may be mentioned are the N-methyl- the N,N-dimethyl-, the N-ethyl-, the N-propyl-, the N,N-diethyl- and the N-isopropylaminocarbonyl radical, of which the N,N-dimethylaminocarbonyl radical is a particular example.

As used herein, Mono- or Di-1-4C-alkylaminosulphonyl stands for a sulphonyl group to which one of the abovementioned mono- or di-1-4C-alkylamino radicals is bonded. Examples which may be mentioned are the methylaminosulphonyl, the dimethylaminosulphonyl and the ethylaminosulphonyl radical, of which the N,N-dimethylaminosulphonyl (dimethylsulphamoyl) radical [(CH₃)₂NS(O)₂-] is a particular example.

As used herein, An 1-4C-Alkylcarbonylamino radical is, for example, the propionylamino (C₂H₅C(O)NH—) and the acetylamino (acetamido) radical (CH₃C(O)NH—).

As used herein, An 1-4C-Alkylsulphonylamino radical is, for example, the ethanesulphonylamino (ethylsulphonylamino) (C₂H₅S(O)₂NH—) and the methanesulphonylamino (methylsulphonylamino) radical (CH₃S(O)₂NH—).

As used herein, 1-4C-Alkylsulfonyl is a sulfonyl group to which one of the abovementioned 1-4C-alkyl radicals is bonded. An example is the methanesulphonyl (methylsulphonyl) radical (CH₃SO₂—).

As used herein, 1-4C-Alkylcarbonyl is a carbonyl group to which one of the abovementioned 1-4C-alkyl radicals is bonded. An example is the acetyl radical (CH₃CO—).

As used herein, Tolyl alone or as part of another group includes o-tolyl, m-tolyl and p-tolyl.

As used herein, Halogen within the meaning of the invention is bromine or, in particular, chlorine or fluorine.

As used herein, Aa1 is a bisaryl radical made up of two aryl groups, which are selected independently from a group consisting of phenyl and naphthyl, and which are linked together via a single bond. Aa1 may include, without being restricted thereto, the biphenyl radical, e.g. the 1,1′-biphenyl-4-yl or 1,1′-biphenyl-3-yl radical.

As used herein, non-limiting examples of R61-substituted derivatives of Aa1 may be mentioned the following radicals:

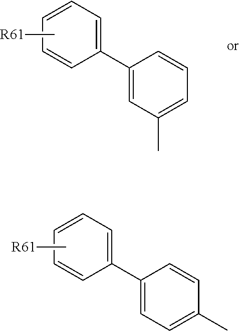

in which the substituent R61 can be attached in the ortho, or, in particular, meta or para position with respect to the binding position in which the benzene ring is bonded to the phenyl radical, such as e.g. 2′-(R61)-1,1′-biphenyl-3-yl, 2′-(R61)-1,1′-biphenyl-4-yl, or, in particular, 3′-(R61)-1,1′-biphenyl-3-yl or 3′-(R61)-1,1′-biphenyl-4-yl, or, yet in particular, 4′-(R61)-1,1′-biphenyl-3-yl or 4′-(R61)-1,1′-biphenyl-4-yl.

As exemplary R61-substituted Aa1 radicals may be more detailed mentioned, for example, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which

R61 is -T2-N(R611)R612, in which
T2 is methylene, dimethylene or trimethylene, and
R611 and R612 together and with inclusion of the nitrogen atom, to which they are bonded, form a morpholino or 4N-methyl-piperazino, or a piperidino or pyrrolidino radical;
such as, for example, any selected from
3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 3′-(morpholin-4-yl-methyl)-biphenyl-4-yl, 4′-(morpholin-4-yl-methyl)-biphenyl-4-yl, 4′-(3-morpholin-4-yl-propyl)-biphenyl-3-yl and 4′-(4-methyl-piperazin-1-ylmethyl)-biphenyl-3-yl.

Yet as exemplary R61-substituted Aa1 radicals may be more detailed mentioned, for example, 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which

R61 is -T2-N(R611)R612, in which T2 is methylene, dimethylene or trimethylene, and
R611 and R612 are both methyl;
such as, for example, any selected from 2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 3′-dimethylaminomethyl-biphenyl-4-yl and 3′-dimethylaminomethyl-biphenyl-3-yl.

Yet as exemplary R61-substituted Aa1 radicals may be more detailed mentioned, for example, 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which

R61 is -T2-N(R611)R612, in which

T2 is methylene, dimethylene or trimethylene, and

R611 is hydrogen, cyclopropyl, cyclopentyl, 2-methoxyethyl, acetyl or methylsulfonyl,

R612 is hydrogen;

for example,

either

R611 is cyclopropyl or 2-methoxy ethyl, and

R612 is hydrogen,

such as, for example, any selected from

4′-(2-methoxy-ethylamino)methyl-biphenyl-3-yl and 4′-cyclopropylaminomethyl-biphenyl-3-yl, or

R611 is hydrogen, cyclopentyl, acetyl or methylsulfonyl, and

R612 is hydrogen,

such as, for example, any selected from

4′-aminomethyl-biphenyl-3-yl, 4′-aminomethyl-biphenyl-4-yl, 4′-(acetylamino)-methyl-biphenyl-4-yl, 4′-(methylsulphonylamino)-methyl-biphenyl-4-yl, 3′-(acetylamino)-methyl-biphenyl-3-yl, 3′-(methylsulphonylamino)-methyl-biphenyl-3-yl and 4′-cyclopentylaminomethyl-biphenyl-4-yl.

Yet as exemplary R61-substituted Aa1 radicals may be more detailed mentioned, for example, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which

R61 is —O-T3-N(R613)R614, in which T3 is dimethylene or trimethylene, and

R613 and R614 together and with inclusion of the nitrogen atom, to which they are bonded, form a morpholino, pyrrolidino or 4N-methyl-piperazino, or a piperidino radical;

such as, for example, any selected from

4′-(2-morpholin-4-yl-ethoxy)-biphenyl-3-yl, 4′-(3-morpholin-4-yl-propoxy)-biphenyl-3-yl, 4′-[2-(4-methyl-piperazin-1-yl)-ethoxy]-biphenyl-3-yl, 4′-(2-pyrrolidin-1-yl-ethoxy]-biphenyl-3-yl, 3′-(3-pyrrolidin-1-yl-propoxy]-biphenyl-4-yl, 4′-(3-pyrrolidin-1-yl-propoxy]-biphenyl-4-yl, 3′-(2-pyrrolidin-1-yl-ethoxy]-biphenyl-4-yl, 4′-(3-morpholin-4-yl-propoxy)-biphenyl-4-yl, 3′-(3-morpholin-4-yl-propoxy)-biphenyl-4-yl, 3′-(2-morpholin-4-yl-ethoxy)-biphenyl-4-yl, 4′-(2-morpholin-4-yl-ethoxy)-biphenyl-4-yl, 4′-[2-(4-methyl-piperazin-1-yl)-ethoxy]-biphenyl-4-yl, 4′-[3-(4-methyl-piperazin-1-yl)-propoxy]-biphenyl-4-yl and 3′-[3-(4-methyl-piperazin-1-yl)-propoxy]-biphenyl-4-yl.

Yet as exemplary R61-substituted Aa1 radicals may be more detailed mentioned, for example, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which

R61 is —O-T5-Het4, in which T5 is a bond, methylene, dimethylene or trimethylene, and

Het4 is 1-methyl-piperidin-4-yl;

such as e.g. 4′-(2-(1-methyl-piperidin-4-yl)-ethoxy)-biphenyl-4-yl.

Yet as exemplary R61-substituted Aa1 radicals may be more detailed mentioned, for example, 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is methylsulphonylamino, N,N-dimethylaminosulphonyl, acetamido, hydroxymethyl, amino, dimethylamino, morpholino, hydroxyl, trifluoromethyl or methoxy;

for example,

either

R61 is methylsulphonylamino, N,N-dimethylaminosulphonyl, acetamido or hydroxymethyl, such as, for example, any selected from 2′-methylsulphonylamino-biphenyl-4-yl, 3′-methylsulphonylamino-biphenyl-4-yl, 4′-methylsulphonylamino-biphenyl-4-yl, 4′-methylsulphonylamino-biphenyl-3-yl, 4′-dimethylsulphamoyl-biphenyl-4-yl, 3′-acetamido-biphenyl-4-yl, 4′-acetamido-biphenyl-4-yl and 3′-hydroxymethyl-biphenyl-4-yl,

or

R61 is amino, dimethylamino, morpholino, hydroxyl, trifluoromethyl or methoxy, such as, for example, any selected from 3′-amino-biphenyl-4-yl, 4′-morpholin-4-yl-biphenyl-4-yl, 4′-hydroxy-biphenyl-4-yl, 3′-trifluoromethyl-biphenyl-4-yl, 3′-dimethylamino-biphenyl-4-yl and 4′-methoxy-biphenyl-4-yl.

Yet as exemplary R61-substituted Aa1 radicals may be more detailed mentioned, for example, 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which R61 is —C(O)—N(H)-T3-N(R613)R614, in which T3 is dimethylene or trimethylene, and

R613 and R614 are both methyl;

such as, for example, any selected from

3′-[(2-dimethylamino-ethylamino)-carbonyl]-biphenyl-4-yl, 4′-[(2-dimethylamino-ethylamino)-carbonyl]-biphenyl-4-yl and 4′-[(2-dimethylamino-ethylamino)-carbonyl]-biphenyl-3-yl.

An example of R61-substituted Aa1 radicals may be 3′-(R61)-1,1′-biphenyl-3-yl, in which R61 is any one selected from the group GABI consisting of 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl.

Another example of R61-substituted Aa1 radicals may be 3′-(R61)-1,1′-biphenyl-4-yl, in which R61 is any one selected from the group GABI given above.

Another example of R61-substituted Aa1 radicals may be 4′-(R61)-1,1′-biphenyl-3-yl, in which R61 is any one selected from the group GABI given above.

Another example of R61-substituted Aa1 radicals may be 4′-(R61)-1,1′-biphenyl-4-yl, in which R61 is any one selected from the group GABI given above.

Specifically, as an exemplary R61-substituted Aa1 radical may be explicitely mentioned, for example, any one selected from 3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 3′-(morpholin-4-yl-methyl)-biphenyl-4-yl, 4′-(morpholin-4-yl-methyl)-biphenyl-4-yl, 4′-(3-morpholin-4-yl-propyl)-biphenyl-3-yl,

4′-(4-methyl-piperazin-1-ylmethyl)-biphenyl-3-yl, 4′-(2-morpholin-4-yl-ethoxy)-biphenyl-3-yl, 4′-(3-morpholin-4-yl-propoxy)-biphenyl-3-yl, 4′-[2-(4-methyl-piperazin-1-yl)-ethoxy]-biphenyl-3-yl, 4′-(2-pyrrolidin-1-yl-ethoxy]-biphenyl-3-yl, 3′-(3-pyrrolidin-1-yl-propoxy]-biphenyl-4-yl, 4′-(3-pyrrolidin-1-yl-propoxy]-biphenyl-4-yl, 3′-(2-pyrrolidin-1-yl-ethoxy]-biphenyl-4-yl, 4′-(3-morpholin-4-yl-propoxy)-biphenyl-4-yl, 3′-(3-morpholin-4-yl-propoxy)-biphenyl-4-yl, 3′-(2-morpholin-4-yl-ethoxy)-biphenyl-4-yl, 4′-(2-morpholin-4-yl-ethoxy)-biphenyl-4-yl, 4′-[2-(4-methyl-piperazin-1-yl)-ethoxy]-biphenyl-4-yl, 4′-[3-(4-methyl-piperazin-1-yl)-propoxy]-biphenyl-4-yl, 3′-[3-(4-methyl-piperazin-1-yl)-propoxy]-biphenyl-4-yl,

4′-(2-(1-methyl-piperidin-4-yl)-ethoxy)-biphenyl-4-yl, 2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 3′-dimethylaminomethyl-biphenyl-4-yl, 3′-dimethylaminomethyl-biphenyl-3-yl,

3′-[(2-dimethylamino-ethylamino)-carbonyl]-biphenyl-4-yl, 4′-[(2-dimethylamino-ethylamino)-carbonyl]-biphenyl-4-yl, 4′-[(2-dimethylamino-ethylamino)-carbonyl]-biphenyl-3-yl, 2′-methylsulphonylamino-biphenyl-4-yl, 3′-methylsulphonylamino-biphenyl-4-yl, 4′-methylsulphonylamino-biphenyl-4-yl, 4′-methylsulphonylamino-biphenyl-3-yl, 4′-dimethylsulphamoyl-biphenyl-4-yl, 3′-acetamido-biphenyl-4-yl, 4′-acetamido-biphenyl-4-yl, 3′-amino-biphenyl-4-yl, 4′-morpholin-4-yl-biphenyl-4-yl, 4′-hydroxy-biphenyl-4-yl, 3′-trifluoromethyl-biphenyl-4-yl and 4′-methoxy-biphenyl-4-yl, 4′-(2-methoxy-ethylamino)methyl-biphenyl-3-yl, 4′-aminomethyl-biphenyl-3-yl, 4′-aminomethyl-biphenyl-4-yl, 4′-(acetylamino)-methyl-biphenyl-4-yl, 4′-(methylsulphonylamino)-methyl-biphenyl-4-yl, 3′-(acetylamino)-methyl-biphenyl-3-yl, 3′-(methylsulphonylamino)-methyl-biphenyl-3-yl, 4′-cyclopentylaminomethyl-biphenyl-4-yl, 4′-cyclopropylaminomethyl-biphenyl-3-yl, and 3′-hydroxymethyl-biphenyl-4-yl.

More specifically, as an exemplary R61-substituted Aa1 radical may be more explicitely mentioned, for example, any one selected from 4′-(2-morpholin-4-yl-ethyl)-biphenyl-3-yl, 4′-(3-morpholin-4-yl-propoxy)-biphenyl-3-yl, 4′-[2-(4-methyl-piperazin-1-yl)-ethoxy]-biphenyl-3-yl, and 4′-dimethylaminomethyl-biphenyl-4-yl.

As used herein, Hh1 is a bisheteroaryl radical made up of two heteroaryl groups, all which are selected independently from a group consisting of monocyclic 5- or 6-membered heteroaryl radicals comprising one or two heteroatoms, each of which is selected from the group consisting of nitrogen, oxygen and sulfur, and which are linked together via a single bond.

Hh1 may include, without being restricted thereto, the bithiophenyl e.g. thiophen-3-yl-thiophenyl or thiophen-2-yl-thiophenyl, bipyridyl, pyrazolyl-pyridinyl e.g. pyrazol-1-yl-pyridinyl or pyrazol-4-yl-pyridinyl like 6-(pyrazol-4-yl)-pyridin-3-yl, imidazolyl-pyridinyl e.g. imidazol-1-yl-pyridinyl, pyrazolyl-thiophenyl e.g. pyrazol-4-yl-thiophenyl like 5-(pyrazol-4-yl)-thiophen-2-yl, or pyridinyl-thiophenyl radical e.g. pyridin-2-yl-thiophenyl, pyridin-3-yl-thiophenyl or pyridin-4-yl-thiophenyl like 5-(pyridin-2-yl)-thiophen-2-yl or 5-(pyridin-4-yl)-thiophen-2-yl, or the thiazolyl-thiophenyl e.g. thiazol-4-yl-thiophenyl like 5-(thiazol-4-yl)-thiophen-2-yl, or thiazolyl-pyridinyl radical like 6-(thiazol-4-yl)-pyridin-3-yl.

In a special detail, exemplary Hh1 radicals may include pyridinyl-thiophenyl, e.g. 5-(pyridin-4-yl)-thiophen-2-yl. In another special detail, exemplary Hh1 radicals may include pyrazolyl-thiophenyl, e.g. 5-(pyrazol-4-yl)-thiophen-2-yl. In another special detail, exemplary Hh1 radicals may include bipyridyl, e.g. 2,4′-bipyridyl-5-yl. In another special detail, exemplary Hh1 radicals may include thiazolyl-thiophenyl, e.g. 5-(thiazol-4-yl)-thiophen-2-yl. In another special detail, exemplary Hh1 radicals may include pyrazolyl-pyridinyl, e.g. 6-(pyrazol-4-yl)-pyridin-3-yl. In another special detail, exemplary Hh1 radicals may include thiazolyl-pyridinyl, e.g. 6-(thiazol-4-yl)-pyridin-3-yl.

As non-limiting example of R61-substituted derivatives of Hh1 may be mentioned [1N-(1-4C-alkyl)-pyrazolyl]-thiophenyl, such as e.g. [1N-(1-4C-alkyl)-pyrazol-4-yl]-thiophenyl, like 5-[1N-(1-2C-alkyl)-pyrazol-4-yl]-thiophen-2-yl, e.g. 5-(1N-methyl-pyrazol-4-yl)-thiophen-2-yl.

Yet as non-limiting example of R61-substituted derivatives of Hh1 may be mentioned [1N-(1-4C-alkyl)-pyrazolyl]-pyridinyl, such as e.g. [1N-(1-4C-alkyl)-pyrazol-4-yl]-pyridinyl or 6-[1N-(1-4C-alkyl)-pyrazolyl]-pyridin-3-yl, like 6-[1N-(1-2C-alkyl)-pyrazol-4-yl]-pyridin-3-yl, e.g. 6-(1N-methyl-pyrazol-4-yl)-pyridin-3-yl.

Yet as non-limiting example of R61-substituted derivatives of Hh1 may be mentioned [(R61)-pyridinyl]-thiophenyl, such as e.g. the following radicals:

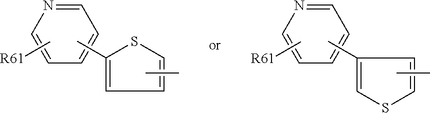

in which the substituent R61 can be attached in the ortho, or, in particular, meta or para position with respect to the binding position in which the pyridinyl ring is bonded to the thiophenyl radical, such as e.g. [2-(R61)-pyridin-4-yl]-thiophenyl or [6-(R61)-pyridin-3-yl]-thiophenyl, like 5-[2-(R61)-pyridin-4-yl]-thiophen-2-yl or 5-[6-(R61)-pyridin-3-yl]-thiophen-2-yl.

Yet as non-limiting example of R61-substituted derivatives of Hh1 may be mentioned [(R61)-thiazolyl]-thiophenyl, such as e.g. the following radicals:

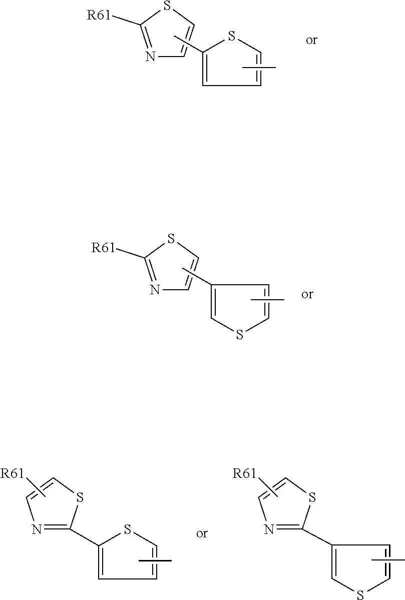

such as e.g. [2-(R61)-thiazol-4-yl]-thiophenyl, like 5-[2-(R61)-thiazol-4-yl]-thiophen-2-yl.

Yet as non-limiting example of R61-substituted derivatives of Hh1 may be mentioned [(R61)-pyridinyl]-pyridinyl, such as e.g. the following radicals:

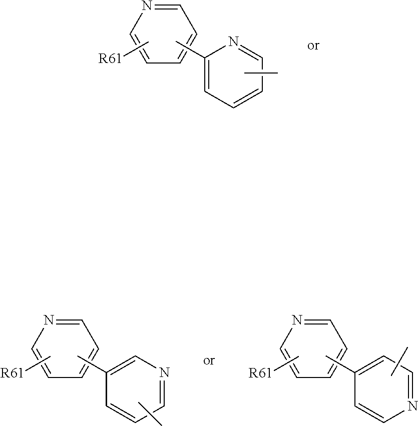

in which the substituent R61 can be attached in the ortho, or, in particular, meta or para position with respect to the binding position in which the terminal pyridinyl ring is bonded to the other pyridinyl radical, such as e.g. [2-(R61)-pyridin-4-yl]-pyridinyl or [6-(R61)-pyridin-3-yl]-pyridinyl or 6-[(R61)-pyridinyl]-pyridin-3-yl, like 6-[2-(R61)-pyridin-4-yl]-pyridin-3-yl [i.e. 2′-(R61)-2,4′-bipyridyl-5-yl] or 6-[6-(R61)-pyridin-3-yl]-pyridin-3-yl [i.e. 6′-(R61)-2,3′-bipyridyl-5-yl].

As exemplary R61-substituted Hh1 radicals may be more detailed mentioned, for example, 5-[2-(R61)-pyridin-4-yl]-thiophen-2-yl or 5-[6-(R61)-pyridin-3-yl]-thiophen-2-yl, in which

R61 is -T2-N(R611)R612, in which T2 is a bond, and

R611 and R612 are both hydrogen, or

R611 and R612 together and with inclusion of the nitrogen atom, to which they are bonded, form a morpholino or 4N-methyl-piperazino, or a piperidino or pyrrolidino radical;

such as e.g. 5-[2-(4-methyl-piperazin-1-yl)-pyridin-4-yl]-thiophen-2-yl.

Yet as exemplary R61-substituted Hh1 radicals may be more detailed mentioned, for example, 2′-(R61)-2,4′-bipyridyl-5-yl or 6′-(R61)-2,3′-bipyridyl-5-yl, in which

R61 is -T2-N(R611)R612, in which T2 is a bond, and

R611 and R612 are both hydrogen, or

R611 and R612 together and with inclusion of the nitrogen atom, to which they are bonded, form a morpholino, 4N-methyl-piperazino, piperidino or pyrrolidino radical;

such as e.g. 2′-(4-methyl-piperazin-1-yl)-2,4′-bipyridyl-5-yl.

Specifically, as an exemplary R61-substituted Hh1 radical may be explicitely mentioned, for example, any one selected from 5-[2-(4-methyl-piperazin-1-yl)-pyridin-4-yl]-thiophen-2-yl, 5-(1N-methyl-pyrazol-4-yl)-thiophen-2-yl, 2′-(4-methyl-piperazin-1-yl)-2,4′-bipyridyl-5-yl, 5-(2-methyl-thiazol-4-yl)-thiophen-2-yl, and 6-(1N-methyl-pyrazol-4-yl)-pyridin-3-yl.

More specifically, as an exemplary R61-substituted Hh1 radical may be more explicitely mentioned, for example, 5-[2-(4-methyl-piperazin-1-yl)-pyridin-4-yl]-thiophen-2-yl.

Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group.

Ah1 may include, without being restricted thereto, the phenyl-thiophenyl e.g. 5-phenyl-thiophen-2-yl, or the phenyl-pyridyl e.g. 6-phenyl-pyridin-3-yl, radical.

In a special detail, exemplary Ah1 radicals may include phenyl-thiophenyl, e.g. 5-(phenyl)-thiophen-2-yl.

Yet in a special detail, exemplary Ah1 radicals may include phenyl-pyridinyl, e.g. 6-(phenyl)-pyridin-3-yl.

As non-limiting example of R61-substituted derivatives of Ah1 may be mentioned [(R61)-phenyl]-thiophenyl, such as e.g. the following radicals:

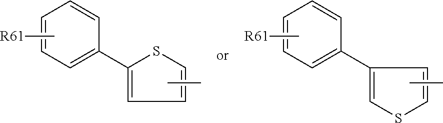

in which the substituent R61 can be attached in the ortho, or, in particular, meta or para position with respect to the binding position in which the phenyl ring is bonded to the thiophenyl radical, such as e.g. [3-(R61)-phenyl]-thiophenyl or [4-(R61)-phenyl]-thiophenyl, like 5-[3-(R61)-phenyl]-thiophen-2-yl or 5-[4-(R61)-phenyl]-thiophen-2-yl.

Yet as non-limiting example of R61-substituted derivatives of Ah1 may be mentioned [(R61 j-phenyl]-pyridinyl, such as e.g. the following radicals:

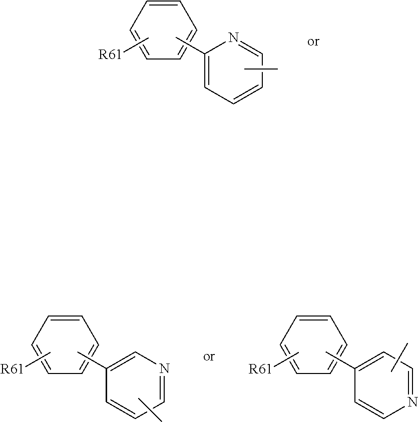

in which the substituent R61 can be attached in the ortho, or, in particular, meta or para position with respect to the binding position in which the phenyl ring is bonded to the pyridinyl radical, such as e.g. [3-(R61)-phenyl]-pyridinyl or [4-(R61)-phenyl]-pyridinyl or 6-[(R61)-phenyl]-pyridin-3-yl, like 6-[3-(R61)-phenyl]-pyridin-3-yl or 6-[4-(R61)-phenyl]-pyridin-3-yl.

As exemplary R61-substituted Ah1 radicals may be more detailed mentioned, for example, 5-[3-(R61)-phenyl]-thiophen-2-yl or 5-[4-(R61)-phenyl]-thiophen-2-yl, in which

R61 is -T2-N(R611)R612, in which T2 is methylene, dimethylene or trimethylene, and

R611 and R612 together and with inclusion of the nitrogen atom, to which they are bonded, form a morpholino or 4N-methyl-piperazino, or a piperidino or pyrrolidino radical;

such as, for example, any selected from 5-[4-(2-morpholin-4-yl-ethyl)-phenyl]-thiophen-2-yl, 5-[4-(morpholin-4-yl-methyl)-phenyl]-thiophen-2-yl and 5-[3-(morpholin-4-yl-methyl)-phenyl]-thiophen-2-yl.

Yet as exemplary R61-substituted Ah1 radicals may be more detailed mentioned, for example, 5-[3-(R61)-phenyl]-thiophen-2-yl or 5-[4-(R61)-phenyl]-thiophen-2-yl, in which

R61 is -T2-N(R611)R612, in which T2 is methylene, dimethylene or trimethylene, and

R611 and R612 are both methyl;

such as, for example, any selected from 5-(4-dimethylaminomethyl-phenyl)-thiophen-2-yl and 5-(3-dimethylaminomethyl-phenyl)-thiophen-2-yl.

Yet as exemplary R61-substituted Ah1 radicals may be more detailed mentioned, for example, 5-[3-(R61)-phenyl]-thiophen-2-yl or 5-[4-(R61)-phenyl]-thiophen-2-yl, in which

R61 is -T2-N(R611)R612, in which T2 is methylene, dimethylene or trimethylene, and

R611 is hydrogen, cyclopropyl, cyclopentyl, 2-methoxyethyl, acetyl or methylsulfonyl, R612 is hydrogen;

such as, for example, any selected from 5-(3-aminomethyl-phenyl)-thiophen-2-yl, 5-[3-(acetylamino)-methyl-phenyl]-thiophen-2-yl and 5-[3-(methylsulphonylamino)-methyl-phenyl]-thiophen-2-yl.

Yet as exemplary R61-substituted Ah1 radicals may be more detailed mentioned, for example, 5-[3-(R61)-phenyl]-thiophen-2-yl or 5-[4-(R61)-phenyl]-thiophen-2-yl, in which
R61 is methylsulphonylamino, N,N-dimethylaminosulphonyl, acetamido, hydroxymethyl, amino, dimethylamino, morpholino, hydroxyl, trifluoromethyl or methoxy;

such as e.g. 5-(4-dimethylsulphamoyl-phenyl)-thiophen-2-yl.

Yet as exemplary R61-substituted Ah1 radicals may be more detailed mentioned, for example, 5-[3-(R61)-phenyl]-thiophen-2-yl or 5-[4-(R61)-phenyl]-thiophen-2-yl, in which

R61 is —O-T3-N(R613)R614, in which T3 is dimethylene or trimethylene, and

R613 and R614 together and with inclusion of the nitrogen atom, to which they are bonded, form a morpholino, pyrrolidino or 4N-methyl-piperazino, or a piperidino radical;

such as, for example, any selected from 5-[4-(2-morpholin-4-yl-ethoxy)-phenyl]-thiophen-2-yl, 5-[4-(3-morpholin-4-yl-propoxy)-phenyl]-thiophen-2-yl, 5-{4-[2-(4-methyl-piperazin-1-yl)-ethoxy]-phenyl}-thiophen-2-yl and 5-[4-(2-pyrrolidin-1-yl-ethoxy)-phenyl]-thiophen-2-yl.

Yet as exemplary R61-substituted Ah1 radicals may be more detailed mentioned, for example, 6-[3-(R61)-phenyl]-pyridin-3-yl or 6-[4-(R61)-phenyl]-pyridin-3-yl, in which

R61 is -T2-N(R611)R612, in which T2 is methylene, dimethylene or trimethylene, and

R611 and R612 are both methyl;

such as, for example, any selected from

6-(4-dimethylaminomethyl-phenyl)-pyridin-3-yl and 6-(3-dimethylaminomethyl-phenyl)-pyridin-3-yl.

Yet as exemplary R61-substituted Ah1 radicals may be more detailed mentioned, for example, 6-[3-(R61)-phenyl]-pyridin-3-yl or 6-[4-(R61)-phenyl]-pyridin-3-yl, in which

R61 is —O-T3-N(R613)R614, in which T3 is dimethylene or trimethylene, and

R613 and R614 together and with inclusion of the nitrogen atom, to which they are bonded, form a morpholino, piperidino, pyrrolidino or 4N-methyl-piperazino radical;

such as e.g. 6-[4-(2-pyrrolidin-1-yl-ethoxy)-phenyl]-pyridin-3-yl.

An example of R61-substituted Ah1 radicals may be [4-(R61)-phenyl]-pyridinyl, e.g. 6-[4-(R61)-phenyl]-pyridin-3-yl, in which R61 is any one selected from the group G_{Ah1} consisting of 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl.

Another example of R61-substituted Ah1 radicals may be [3-(R61)-phenyl]-pyridinyl, e.g. 6-[3-(R61)-phenyl]-pyridin-3-yl, in which R61 is any one selected from the group G_{Ah1} given above. A further example of R61-substituted Ah1 radicals may be [4-(R61)-phenyl]-thiophenyl, e.g. 5-[4-(R61)-phenyl]-thiophen-2-yl, in which R61 is any one selected from the group G_{Ah1} given above. Another example of R61-substituted Ah1 radicals may be [3-(R61)-phenyl]-thiophenyl, e.g. 5-[3-(R61)-phenyl]-thiophen-2-yl, in which R61 is any one selected from the group G_{Ah1} given above.

Specifically, as an exemplary R61-substituted Ah1 radical may be explicitely mentioned, for example, any one selected from 5-[4-(2-morpholin-4-yl-ethyl)-phenyl]-thiophen-2-yl, 5-[4-(morpholin-4-yl-methyl)-phenyl]-thiophen-2-yl, 5-[3-(morpholin-4-yl-methyl)-phenyl]-thiophen-2-yl, 5-[4-(2-morpholin-4-yl-ethoxy)-phenyl]-thiophen-2-yl, 5-[4-(3-morpholin-4-yl-propoxy)-phenyl]-thiophen-2-yl, 5-{4-[2-(4-methyl-piperazin-1-yl)-ethoxy]-phenyl}-thiophen-2-yl, 5-[4-(2-pyrrolidin-1-yl-ethoxy)-phenyl]-thiophen-2-yl, 5-(4-dimethylaminomethyl-phenyl)-thiophen-2-yl, 5-(3-dimethylaminomethyl-phenyl)-thiophen-2-yl, 6-(4-dimethylaminomethyl-phenyl)-pyridin-3-yl, 6-(3-dimethylaminomethyl-phenyl)-pyridin-3-yl, and

6-[4-(2-pyrrolidin-1-yl-ethoxy)-phenyl]-pyridin-3-yl, 5-(3-aminomethyl-phenyl)-thiophen-2-yl, 5-[3-(acetylamino)-methyl-phenyl]-thiophen-2-yl, 5-[3-(methylsulphonylamino)-methyl-phenyl]-thiophen-2-yl, and 5-(4-dimethylsulphamoyl-phenyl)-thiophen-2-yl.

More specifically, as an exemplary R61-substituted Ah1 radical may be more explicitely mentioned, for example, 5-(4-dimethylaminomethyl-phenyl)-thiophen-2-yl.

It is to be stated, that each of the radicals Hh1 and Ah1 is bonded via a ring carbon atom to the moiety T1.

Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group.

A particular embodiment of said Ha1 radicals refers to heteroaryl-phenyl radicals, particularly 3-(heteroaryl)-phenyl or 4-(heteroaryl)-phenyl radicals.

Ha1 may include, without being restricted thereto, the furanyl-phenyl, thiophenyl-phenyl, pyrazolyl-phenyl e.g. pyrazol-1-yl-phenyl or pyrazol-4-yl-phenyl, imidazolyl-phenyl e.g. imidazol-1-yl-phenyl, isoxazolyl-phenyl, or pyridinyl-phenyl radicals, or the thiazolyl-phenyl e.g. thiazol-4-yl-phenyl radical.

In a special detail, exemplary Ha1 radicals may include pyrazolyl-phenyl, e.g. 3-(pyrazolyl)-phenyl or 4-(pyrazolyl)-phenyl. Yet in a special detail, exemplary Ha1 radicals may include pyridinyl-phenyl, e.g. 4-(pyridinyl)-phenyl or 3-(pyridinyl)-phenyl. Yet in a special detail, exemplary Ha1 radicals may include isoxazolyl-phenyl, e.g. 4-(isoxazolyl)-phenyl or 3-(isoxazolyl)-phenyl. Yet in a special detail, exemplary Ha1 radicals may include thiazolyl-phenyl, e.g. 4-(thiazolyl)-phenyl or 3-(thiazolyl)-phenyl.

In a further special detail, exemplary Ha1 radicals may include 3-(pyrazol-1-yl)-phenyl, 4-(pyrazol-1-yl)-phenyl, 4-(pyridin-4-yl)-phenyl, 3-(pyridin-4-yl)-phenyl, 4-(pyridin-3-yl)-phenyl, 3-(pyridin-3-yl)-phenyl, 4-(isoxazol-4-yl)-phenyl, 3-(isoxazol-4-yl)-phenyl, 3-(pyrazol-4-yl)-phenyl or 4-(pyrazol-4-yl)-phenyl.

As non-limiting example of R61-substituted derivatives of Ha1 may be mentioned [1N-(1-4C-alkyl)-pyrazolyl]-phenyl, such as e.g. [1N-(1-4C-alkyl)-pyrazol-4-yl]-phenyl, like 3-[1N-(1-2C-alkyl)-pyrazol-4-yl]-phenyl or 4-[1N-(1-2C-alkyl)-pyrazol-4-yl]-phenyl, e.g. 3-(1N-methyl-pyrazol-4-yl)-phenyl or 4-(1N-methyl-pyrazol-4-yl)-phenyl.

As non-limiting example of R61- and/or R62-substituted derivatives of Ha1 may be mentioned (methyl-isoxazolyl)-phenyl or (dimethyl-isoxazolyl)-phenyl, such as e.g. 3-(3,5-dimethyl-isoxazol-4-yl)-phenyl or 4-(3,5-dimethyl-isoxazol-4-yl)-phenyl.

Yet as non-limiting example of R61-substituted derivatives of Ha1 may be mentioned [(R61)-pyridinyl]-phenyl, such as e.g. the following radicals:

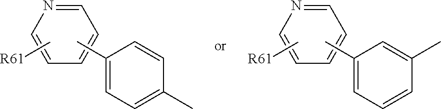

in which the substituent R61 can be attached in the ortho, or, in particular, meta or para position with respect to the binding position in which the pyridinyl ring is bonded to the phenyl radical, such as e.g. 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl.

As exemplary R61-substituted Ha1 radicals may be more detailed mentioned, for example, 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl, in which

R61 is -T2-N(R611)R612, in which T2 is a bond, and

R611 and R612 together and with inclusion of the nitrogen atom, to which they are bonded, form a morpholino or 4N-methyl-piperazino, or a piperidino or pyrrolidino radical;

such as, for example, any selected from 4-[2-(4-methyl-piperazin-1-yl)-pyridin-4-yl]-phenyl and 3-[2-(4-methyl-piperazin-1-yl)-pyridin-4-yl]-phenyl.

Yet as exemplary R61-substituted Ha1 radicals may be more detailed mentioned, for example, 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl, in which

R61 is -T2-N(R611)R612, in which T2 is a bond, and

R611 and R612 are both hydrogen;

such as, for example, any selected from 4-[6-amino-pyridin-3-yl]-phenyl and 3-[6-amino-pyridin-3-yl]-phenyl.

Yet as exemplary R61-substituted Ha1 radicals may be more detailed mentioned, for example, 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl, in which R61 is methoxy; such as, for example, any selected from 4-[6-methoxy-pyridin-3-yl]-phenyl and 3-[6-methoxy-pyridin-3-yl]-phenyl.

Specifically, as an exemplary R61-substituted Ha1 radical may be explicitely mentioned, for example, any one selected from 4-[2-(4-methyl-piperazin-1-yl)-pyridin-4-yl]-phenyl, 3-[2-(4-methyl-piperazin-1-yl)-pyridin-4-yl]-phenyl, 4-[6-amino-pyridin-3-yl]-phenyl, 3-[6-amino-pyridin-3-yl]-phenyl, 4-[6-methoxy-pyridin-3-yl]-phenyl, 3-[6-methoxy-pyridin-3-yl]-phenyl, 3-(1N-methyl-pyrazol-4-yl)-phenyl, 4-(1N-methyl-pyrazol-4-yl)-phenyl, and 4-(3,5-dimethyl-isoxazol-4-yl)-phenyl.

More specifically, as an exemplary R61-substituted Ha1 radical may be more explicitely mentioned, for example, any one selected from 4-[2-(4-methyl-piperazin-1-yl)-pyridin-4-yl]-phenyl, 3-[2-(4-methyl-piperazin-1-yl)-pyridin-4-yl]-phenyl, 4-[6-amino-pyridin-3-yl]-phenyl, and 4-(1N-methyl-pyrazol-4-yl)-phenyl.

As part of the radicals Hh1, Ah1 and Ha1, the mentioned heteroaryl group selected from a group consisting of monocyclic 5- or 6-membered heteroaryl radicals comprising one or two heteroatoms, each of which is selected from the group consisting of nitrogen, oxygen and sulphur, may be choosen, for example, from the group consisting of, the 5-membered heteroaryl radicals, pyrrolyl, furanyl, thiophenyl, oxazolyl, isoxazolyl, thiazolyl, isothiazolyl, imidazolyl and pyrazolyl, and, the 6-membered heteroaryl radicals, pyridinyl, pyrimidinyl, pyrazinyl and pyridazinyl.

Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the to the parent molecular group.

A particular embodiment of said Ha2 radicals refers to heteroaryl-phenyl radicals, particularly 3-(heteroaryl)-phenyl or 4-(heteroaryl)-phenyl radicals.

Another particular embodiment of said Ha2 radicals refers to heteroaryl-phenyl radicals, particularly 3-(heteroaryl)-phenyl or 4-(heteroaryl)-phenyl radicals, in which the heteroaryl moiety contains a benzene ring.

Another particular embodiment of said Ha2 radicals refers to heteroaryl-phenyl radicals, particularly 3-(heteroaryl)-phenyl or 4-(heteroaryl)-phenyl radicals, in which the heteroaryl moiety contains a benzene ring, and whereby the heteroaryl moiety is attached via said benzene ring to the phenyl moiety.

Ha2 may include, without being restricted thereto, the indolyl-phenyl, benzothiophenyl-phenyl, benzofuranyl-phenyl, benzoxazolyl-phenyl, benzothiazolyl-phenyl, indazolyl-phenyl, benzimidazolyl-phenyl, benzisoxazolyl-phenyl, benzisothiazolyl-phenyl, benzofurazanyl-phenyl, benzotriazolyl-phenyl, benzothiadiazolyl-phenyl, quinolinyl-phenyl, isoquinolinyl-phenyl, quinazolinyl-phenyl, quinoxalinyl-phenyl, cinnolinyl-phenyl, indolizinyl-phenyl or naphthyridinyl-phenyl.

In a special detail, exemplary Ha2 radicals may include 3-(indolyl)-phenyl or 4-(indolyl)-phenyl. In a further special detail, exemplary Ha2 radicals may include 3-(indol-5-yl)-phenyl or 4-(indol-5-yl)-phenyl.

Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,

A particular embodiment of said Ha3 radicals refers to heteroaryl-phenyl radicals, particularly 3-(heteroaryl)-phenyl or 4-(heteroaryl)-phenyl radicals. Ha3 may include, without being restricted thereto, the thiadiazolyl-phenyl (e.g. [1,3,4]thiadiazol-2-yl-phenyl or [1,2,5]thiadiazol-3-yl-phenyl), oxadiazolyl-phenyl (e.g. [1,3,4]oxadiazol-2-yl-phenyl or [1,2,4]oxadiazol-5-yl-phenyl), triazolyl-phenyl (e.g. triazol-1-yl-phenyl or [1,2,3]triazol-4-yl) or tetrazolyl-phenyl (e.g. tetrazol-1-yl-phenyl or tetrazol-5-yl-phenyl) radicals.

In a special detail, exemplary Ha3 radicals may include triazolyl-phenyl, e.g. 3-(triazolyl)-phenyl or 4-(triazolyl)-phenyl. In a further special detail, exemplary Ha3 radicals may include 3-[1,2,3]triazol-4-yl-phenyl or 4-[1,2,3]triazol-4-yl-phenyl.

As non-limiting example of R61-substituted derivatives of Ha3 may be mentioned {1N—(R61)-[1,2,3]triazolyl}-phenyl, such as e.g. {1N—(R61)-[1,2,3]triazol-4-yl}-phenyl, like 3-{1N—(R61)-[1,2,3]triazol-4-yl}-phenyl or 4-{1N—(R61)-[1,2,3]triazol-4-yl}-phenyl.

As exemplary R61-substituted Ha3 radicals may be more detailed mentioned, for example, 3-[1N—(R61)-1,2,3-triazol-4-yl]-phenyl or 4-{1N—(R61)-[1,2,3]triazol-4-yl}-phenyl, in which

R61 is -T2-N(R611)R612, in which

T2 is dimethylene or trimethylene, and

R611 and R612 together and with inclusion of the nitrogen atom, to which they are bonded, form a piperidino, pyrrolidino, morpholino or 4N-methyl-piperazino radical;

such as e.g. 4-{1-(2-morpholin-4-yl-ethyl)-[1,2,3]triazol-4-yl}-phenyl or 4-{1-(2-piperidin-1-yl-ethyl)-[1,2,3]triazol-4-yl}-phenyl.

Ha4 is a heteroarylaryl radical made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby

Ha4 is bonded via said aryl moiety to the to the parent molecular group,

A particular embodiment of said Ha4 radicals refers to heteroaryl-phenyl radicals, particularly 3-(heteroaryl)-phenyl or 4-(heteroaryl)-phenyl radicals.

Another particular embodiment of said Ha4 radicals refers to heteroaryl-phenyl radicals, particularly 3-(heteroaryl)-phenyl or 4-(heteroaryl)-phenyl radicals, whereby the heteroaryl moiety is attached via its benzene ring to the phenyl moiety.

Ha4 may include, without being restricted thereto, the indolinyl-phenyl, isoindolinyl-phenyl, (1,2,3,4-tetrahydroquinolinyl)-phenyl or (1,2,3,4-tetrahydroisoquinolinyl)-phenyl, (2,3-dihydrobenzofuranyl)-phenyl, (2,3-dihydrobenzothiophenyl)-phenyl, (benzo[1,3]dioxolyl)-phenyl, (2,3-dihydrobenzo[1,4]dioxinyl)-phenyl, chromanyl-phenyl, chromenyl-phenyl or (2,3-dihydrobenzo[1,4]oxazinyl)-phenyl.

In a special detail, exemplary Ha4 radicals may include (benzo[1,3]dioxolyl)-phenyl, e.g. 3-(benzo[1,3]dioxolyl)-phenyl or 4-(benzo[1,3]dioxolyl)-phenyl, such as, for example, (benzo[1,3]dioxol-5-yl)-phenyl, e.g. 3-(benzo[1,3]dioxol-5-yl)-phenyl or 4-(benzo[1,3]dioxol-5-yl)-phenyl. Yet in a special detail, exemplary Ha4 radicals may include (2,3-dihydrobenzofuranyl)-phenyl, e.g. 3-(2,3-dihydrobenzofuranyl)-phenyl or 4-(2,3-dihydrobenzofuranyl)-phenyl, such as, for example, (2,3-dihydrobenzofuran-5-yl)-phenyl or (2,3-dihydrobenzofuran-6-yl)-phenyl, e.g. 3-(2,3-dihydrobenzofuran-5-yl)-phenyl or 4-(2,3-dihydrobenzofuran-5-yl)-phenyl. In a further special detail, exemplary Ha4 radicals may include 4-(2,3-dihydrobenzofuran-5-yl)-phenyl.

Har2 stands for a monocyclic 5- or 6-membered unsaturated heteroaromatic ring comprising one to three heteroatoms, each of which is selected from the group consisting of nitrogen, oxygen and sulfur.

Har2 may include, without being restricted thereto, thiophene, oxazole, isoxazole, thiazole, isothiazole, imidazole, pyrazole, triazole, thiadiazole, oxadiazole, pyridine, pyrimidine, pyrazine or pyridazine.

In a special detail, an exemplary Har2 radical may be pyridine.

As used herein, Cycl stands for a ring system of formula Ia, which is bonded to the nitrogen atom of the carboxamide group via the moiety A. Cycl may include, without being restricted thereto, 2-aminophenyl substituted by R71 and/or R72. In a special detail, an exemplary Cycl radical may be 2-aminophenyl.

As used herein, naphthyl, alone or as part of another group, includes naphthalen-1-yl and naphthalen-2-yl.

In the meaning of the present invention, it is to be understood, that, when two structural portions of the compounds of formula (I) are linked via a constituent which has the meaning “bond”, then said two portions are directly attached to another via a single bond.

When R61 has the meaning of —U-T3-N(R613)R614, in which U stands for —C(O)NH—, then R61 is the radical —C(O)NH-T3-N(R613)R614.

As it is known for the skilled person, the expressions morpholino, 4N-(1-4C-alkyl)-piperazino, pyrrolidino and the like stand for morpholin-4-yl, 4N-(1-4C-alkyl)-piperazin-1-yl, pyrrolidin-1-yl and the like, respectively.

In general, unless otherwise mentioned the heterocyclic groups mentioned herein refer to all of the possible isomeric forms thereof. The heterocyclic groups mentioned herein refer, unless otherwise noted, in particular to all of the possible positional isomers thereof. Thus, for example, the term pyridyl or pyridinyl, alone or as part of another group, includes pyridin-2-yl, pyridin-3-yl and pyridin-4-yl.

Constituents which are optionally substituted as stated herein, may be substituted, unless otherwise noted, at any possible position.

The carbocyclic groups, alone or as part of other groups, mentioned herein may be substituted by their given substituents or parent molecular groups, unless otherwise noted, at any substitutable ring carbon atom.

The heterocyclic groups, alone or as part of other groups, mentioned herein may be substituted by their given substituents or parent molecular groups, unless otherwise noted, at any possible position, such as e.g. at any substitutable ring carbon or ring nitrogen atom.

Rings containing quaternizable imino-type ring nitrogen atoms (—N═) may be particularly not quaternized on these imino-type ring nitrogen atoms by the mentioned substituents or parent molecular groups.

Any heteroatom of a heterocyclic ring with unsatisfied valences mentioned herein is assumed to have the hydrogen atom(s) to satisfy the valences.

When any variable occurs more than one time in any constituent, each definition is independent.

According to expert's knowledge the compounds of formula I of the invention as well as their salts may contain, e.g. when isolated in crystalline form, varying amounts of solvents. Included within the scope of the invention are therefore all solvates and in particular all hydrates of the compounds of formula I as well as all solvates and in particular all hydrates of the salts of the compounds of formula I.

The substituents R61 and R62 of compounds of formula I can be attached in any possible position of the Aa1, Hh1, Ha1, Ha2, Ha3, Ha4 or Ah1 radical, whereby emphasis is given to the attachment at the terminal ring; in another embodiment, Q1 is monosubstituted by R61, and is Aa1, Hh1, Ha1 or Ah1, whereby emphasis is given to the attachment of R61 at the terminal ring; in yet another embodiment, R6 is Aa1, Ha1 or Ha2, each of which is monosubstituted by R61, whereby emphasis is given to the attachment of R61 at the terminal ring; in yet another embodiment, R6 is Aa1, Hh1, Ha1, Ha2 or Ah1, each of which is monosubstituted by R61, whereby emphasis is given to the attachment of R61 at the terminal ring; in yet another embodiment, R6 is Aa1, Hh1, Ha1, Ha2, Ha3 or Ah1, each of which is monosubstituted by R61, whereby emphasis is given to the attachement of R61 at the terminal ring; in yet another embodiment, R6 is Ha2, Ha3 or Ha4, each of which is unsubstituted.

Within the meaning of this invention, the terminal ring of Aa1, Hh1, Ha1, Ha2, Ha3, Ha4 or Ah1 refers to those ring portions of these radicals which is not directly attached to the T1 moiety.

The person skilled in the art is aware on account of his/her expert knowledge that certain combinations of the variable characteristics mentioned in the description of this invention may lead to chemically les stable compounds. This can apply, for example, to certain compounds, in which-in a manner being disadvantageous for chemical stability—two heteroatoms (S, N or O) would directly meet or would only be separated by one carbon atom. Particularly, the compounds according to this invention are those, in which the combination of the abovementioned variable substituents does not lead to chemically less stable compounds.

Compounds according to aspect A of the present invention more worthy to be mentioned are those compounds of formula I in which

R1, R2, R3, R4 and R5 are independently hydrogen or 1-4C-alkyl,

R6 is -T1-Q1, in which T1 is a bond,

either

Q1 is substituted by R61 and/or R62, and is Aa1, Hh1, Ha1, Ha2, Ha3, Ha4 or Ah1,

Or Q1 is unsubstituted, and is Ha2, Ha3 or Ha4,

in which
R61 is 1-4C-alkyl, 1-4C-alkoxy, hydroxyl, trifluoromethyl, halogen, hydroxy-1-4C-alkyl, 1-4C-alkylsulphonylamino, tolylsulphonylamino, phenylsulphonylamino, 1-4C-alkylcarbonylamino, di-1-4C-alkylaminosulphonyl, -T2-N(R611)R612, —U-T3-N(R613)R614, -T4-Het3, or —V-T5-Het4, in which T2 is a bond or 1-4C-alkylene,
R611 is hydrogen, 1-4C-alkyl, 3-7C-cycloalkyl, 1-4C-alkoxy-2-4C-alkyl, 1-4C-alkylcarbonyl, or 1-4C-alkylsulphonyl,
R612 is hydrogen or 1-4C-alkyl,
or R611 and R612 together and with inclusion of the nitrogen atom, to which they are bonded, form a heterocyclic ring Het1, in which
Het1 is morpholino, piperidino, pyrrolidino, piperazino or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl, 1-4C-alkoxy-2-4C-alkyl, 1-4C-alkylcarbonyl, or 1-4C-alkylsulphonyl,
R614 is hydrogen or 1-4C-alkyl,
or R613 and R614 together and with inclusion of the nitrogen atom, to which they are bonded, form a heterocyclic ring Het2, in which
Het2 is morpholino, piperidino, pyrrolidino, piperazino or 4N-(1-4C-alkyl)-piperazino,
T4 is a bond or 1-4C-alkylene,
Het3 is 1N-(1-4C-alkyl)-piperidinyl or 1N-(1-4C-alkyl)-pyrrolidinyl,
V is —O— (oxygen) or —C(O)NH—,
T5 is a bond, or 1-4C-alkylene,
Het4 is 1N-(1-4C-alkyl)-piperidinyl or 1N-(1-4C-alkyl)-pyrrolidinyl,
R62 is 1-4C-alkyl,
Aa1 is biphenyl,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an phenyl-heteroaryl radical made up of an phenyl group and a heteroaryl group selected from a group consisting of monocyclic 5- or 6-membered heteroaryl radicals comprising one or two heteroatoms, each of which is selected from the group consisting of nitrogen, oxygen and sulfur, whereby said phenyl and heteroaryl groups are linked together via a single bond, and whereby Ah1 is bonded via said heteroaryl moiety to the parent molecular group,
Ha1 is a heteroaryl-phenyl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a heteroaryl-phenyl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
Ha3 is a heteroaryl-phenyl radical made up of a heteroaryl group selected from a group consisting of monocyclic 5-membered heteroaryl radicals comprising three or four heteroatoms, each of which is selected from the group consisting of nitrogen, oxygen and sulfur, and a phenyl group, whereby said heteroaryl and phenyl groups are linked together via a single bond, and whereby Ha3 is bonded via said phenyl moiety to the to the parent molecular group,
Ha4 is a heteroaryl-phenyl radical made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and a phenyl group, whereby said heteroaryl and phenyl groups are linked together via a single bond, and whereby Ha4 is bonded via said phenyl moiety to the to the parent molecular group,
R7 is hydroxyl, or 2-aminophenyl,
   and the salts of these compounds.

Compounds according to aspect A of the present invention in particular worthy to be mentioned are those compounds of formula I in which
R1, R2, R3, R4 and R5 are hydrogen,
R6 is -T1-Q1, in which T1 is a bond,
either
Q1 is substituted by R61 and/or R62 on the terminal ring, and is Aa1, Hh1, Ha1, Ha2, Ha3, Ha4 or Ah1,
Or Q1 is unsubstituted, and is Ha2, Ha3 or Ha4,
in which
R61 is 1-2C-alkyl, 1-2C-alkoxy, hydroxyl, trifluoromethyl, halogen, hydroxy-1-2C-alkyl, 1-2C-alkylsulphonylamino, 1-2C-alkylcarbonylamino, di-1-2C-alkylaminosulphonyl, -T2-N(R611)R612, —U-T3-N(R613)R614, -T4-Het3, or —V-T5-Het4, in which
T2 is a bond or straight chain 1-4C-alkylene,
R611 is hydrogen, 1-2C-alkyl, 3-5C-cycloalkyl, 1-2C-alkoxy-2-3C-alkyl, 1-2C-alkylcarbonyl, or 1-2C-alkylsulphonyl,
R612 is hydrogen or 1-2C-alkyl,
or R611 and R612 together and with inclusion of the nitrogen atom, to which they are bonded, form a heterocyclic ring Het1, in which
Het1 is morpholino, piperidino, pyrrolidino, piperazino or 4N-(1-2C-alkyl)-piperazino,
U is —O— (oxygen) or —C(O)NH—,
T3 is straight chain 2-4C-alkylene,
R613 is hydrogen, 1-2C-alkyl, 3-5C-cycloalkyl, 1-2C-alkoxy-2-3C-alkyl, 1-2C-alkylcarbonyl, or 1-2C-alkylsulphonyl,
R614 is hydrogen or 1-2C-alkyl,
or R613 and R614 together and with inclusion of the nitrogen atom, to which they are bonded, form a heterocyclic ring Het2, in which
Het2 is morpholino, piperidino, pyrrolidino, piperazino or 4N-(1-2C-alkyl)-piperazino,
T4 is a bond or straight chain 1-4C-alkylene,
Het3 is 1N-(1-2C-alkyl)-piperidinyl or 1N-(1-2C-alkyl)-pyrrolidinyl,
V is —O— (oxygen) or —C(O)NH—,
T5 is a bond or straight chain 1-4C-alkylene,
Het4 is 1N-(1-2C-alkyl)-piperidinyl or 1N-(1-2C-alkyl)-pyrrolidinyl,
R62 is 1-2C-alkyl,
Aa1 is 1,1′-biphenyl-3-yl or 1,1′-biphenyl-4-yl,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an phenyl-heteroaryl radical made up of an phenyl group and a heteroaryl group selected from a group consisting of monocyclic 5- or 6-membered heteroaryl radicals comprising one or two heteroatoms, each of which is selected from the group consisting of nitrogen, oxygen and sulfur, whereby said phenyl and heteroaryl groups are linked together via a single bond, and whereby Ah1 is bonded via said heteroaryl moiety to the parent molecular group,
Ha1 is a 3-(heteroaryl)-phenyl or 4-(heteroaryl)-phenyl radical each made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a 3-(heteroaryl)-phenyl or 4-(heteroaryl)-phenyl radical each made up of a heteroaryl group selected from a group consisting of fused bicyclic 9- or 10-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
Ha3 is a 3-(heteroaryl)-phenyl or 4-(heteroaryl)-phenyl radical each made up of a heteroaryl group selected from a group consisting of monocyclic 5-membered heteroaryl radicals comprising three or four heteroatoms, each of which is selected from the group consisting of nitrogen, oxygen and sulfur, and a phenyl group, whereby said heteroaryl and phenyl groups are linked together via a single bond, and whereby Ha3 is bonded via said phenyl moiety to the to the parent molecular group,
Ha4 is a 3-(heteroaryl)-phenyl or 4-(heteroaryl)-phenyl radical each made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and a phenyl group, whereby said heteroaryl and phenyl groups are linked together via a single bond, and whereby Ha4 is bonded via said phenyl moiety to the to the parent molecular group,
R7 is hydroxyl, or 2-aminophenyl,
   and the salts of these compounds.

Compounds according to aspect A of the present invention in more particular worthy to be mentioned are those compounds of formula I in which
R1, R2, R3, R4 and R5 are hydrogen,
R6 is -T1-Q1, in which T1 is a bond, either
Q1 is substituted by R61 and/or R62 on the terminal ring, and is Aa1, Hh1, Ha1, Ha2, Ha3, Ha4 or Ah1,
or Q1 is unsubstituted, and is Ha2, Ha3 or Ha4,
in which
R61 is methyl, methoxy, hydroxyl, trifluoromethyl, hydroxymethyl, methylsulphonylamino, methylcarbonylamino, dimethylaminosulphonyl, -T2-N(R611)R612, —U-T3-N(R613)R614, -T4-Het3, or —V-T5-Het4, in which
T2 is a bond, methylene, dimethylene or trimethylene,
R611 is hydrogen, methyl, cyclopropyl, cyclopentyl, 2-methoxyethyl, acetyl or methylsulphonyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino, piperazino or 4-methyl-piperazino,
U is —O— (oxygen) or —C(O)NH—,
T3 is dimethylene or trimethylene,
R613 is hydrogen, methyl, cyclopropyl, cyclopentyl, 2-methoxyethyl, acetyl or methylsulphonyl,
R614 is hydrogen or methyl,
or R613 and R614 together and with inclusion of the nitrogen atom, to which they are bonded, form a heterocyclic ring Het2, in which
Het2 is morpholino, piperidino, pyrrolidino, piperazino or 4-methyl-piperazino,
T4 is a bond, methylene, dimethylene or trimethylene,
Het3 is 1-methyl-piperidinyl or 1-methyl-pyrrolidinyl,
V is —O— (oxygen) or —C(O)NH—,
T5 is a bond, methylene, dimethylene or trimethylene,
Het4 is 1-methyl-piperidinyl or 1-methyl-pyrrolidinyl,
R62 is methyl,
Aa1 is 1,1′-biphenyl-3-yl, or 1,1′-biphenyl-4-yl,
Hh1 is a bisheteroaryl radical made up of two heteroaryl groups, which are selected independently from a group consisting of pyrrolyl, furanyl, thiophenyl, oxazolyl, isoxazolyl, thiazolyl, isothiazolyl, imidazolyl, pyrazolyl, pyridinyl, pyrimidinyl, pyrazinyl and pyridazinyl, and
   which are linked together via a single bond,
such as, for example,
Hh1 is pyridinyl-thiophenyl, thiazolyl-thiophenyl, pyrazolyl-thiophenyl, bipyridyl, pyrazolyl-pyridinyl, or thiazolyl-pyridinyl,
Ah1 is a phenyl-heteroaryl radical made up of an phenyl group and a heteroaryl group selected from a group consisting of pyrrolyl, furanyl, thiophenyl, oxazolyl, isoxazolyl, thiazolyl, isothiazolyl, imidazolyl, pyrazolyl, pyridinyl, pyrimidinyl, pyrazinyl and pyridazinyl, whereby said phenyl and heteroaryl groups are linked together via a single bond, and whereby Ah1 is bonded via said heteroaryl moiety to the parent molecular group,
such as, for example,
Ah1 is phenyl-thiophenyl, or phenyl-pyridinyl,
Ha1 is a 3-(heteroaryl)-phenyl or 4-(heteroaryl)-phenyl radical each made up of a heteroaryl group selected from a group consisting of pyrrolyl, furanyl, thiophenyl, oxazolyl, isoxazolyl, thiazolyl, isothiazolyl, imidazolyl, pyrazolyl, pyridinyl, pyrimidinyl, pyrazinyl and pyridazinyl, and a phenyl group, whereby said heteroaryl and phenyl groups are linked together via a single bond, and whereby Ha1 is bonded via said phenyl moiety to the to the parent molecular group,
such as, for example,
Ha1 is 3-(pyridinyl)-phenyl, 3-(thiazolyl)-phenyl, 3-(pyrazolyl)-phenyl, 3-(isoxazolyl)-phenyl, 4-(pyridinyl)-phenyl, 4-(thiazolyl)-phenyl, 4-(pyrazolyl)-phenyl, or 4-(isoxazolyl)-phenyl,
Ha2 is a 3-(heteroaryl)-phenyl or 4-(heteroaryl)-phenyl radical each made up of a heteroaryl group selected from a group consisting of indolyl, benzothiophenyl, benzofuranyl, benzoxazolyl, benzothiazolyl, indazolyl, benzimidazolyl, benzisoxazolyl, benzisothiazolyl, benzofurazanyl, benzotriazolyl, benzothiadiazolyl, quinolinyl, isoquinolinyl, quinazolinyl, quinoxalinyl, cinnolinyl, indolizinyl and naphthyridinyl, and a phenyl group, whereby said heteroaryl and phenyl groups are linked together via a single bond, and whereby Ha2 is bonded via said phenyl moiety to the to the parent molecular group,
such as, for example,
Ha2 is 3-(indolyl)-phenyl, or 4-(indolyl)-phenyl,
Ha3 is a 3-(heteroaryl)-phenyl or 4-(heteroaryl)-phenyl radical each made up of a heteroaryl group selected from a group consisting of thiadiazolyl, oxadiazolyl, triazolyl and tetrazolyl, and a phenyl group, whereby said heteroaryl and phenyl groups are linked together via a single bond, and whereby Ha3 is bonded via said phenyl moiety to the to the parent molecular group,
such as, for example,
Ha3 is 3-(triazolyl)-phenyl, or 4-(triazolyl)-phenyl,
Ha4 is a 3-(heteroaryl)-phenyl or 4-(heteroaryl)-phenyl radical each made up of a heteroaryl group selected from a group consisting of indolinyl, isoindolinyl, 1,2,3,4-tetrahydroquinolinyl, 1,2,3,4-tetrahydroisoquinolinyl, 2,3-dihydrobenzofuranyl, 2,3-dihydrobenzothiophenyl, benzo[1,3]dioxolyl, 2,3-dihydrobenzo[1,4]dioxinyl, chromanyl, chromenyl and 2,3-dihydrobenzo[1,4]oxazinyl, and a phenyl group, whereby said heteroaryl and phenyl groups are linked together via a single bond, and whereby Ha3 is bonded via said phenyl moiety to the to the parent molecular group,
such as, for example,
Ha4 is 3-(benzo[1,3]dioxolyl)-phenyl, 4-(benzo[1,3]dioxolyl)-phenyl, 3-(2,3-dihydrobenzofuranyl)-phenyl, or 4-(2,3-dihydrobenzofuranyl)-phenyl,
R7 is hydroxyl, or 2-aminophenyl,
and the salts of these compounds.

Compounds according to aspect A of the present invention to be emphasized are those compounds of formula I in which
R1, R2, R3, R4 and R5 are hydrogen,
R6 is -T1-Q1, in which T1 is a bond;
either
Q1 is substituted by R61 on the terminal ring, and is Aa1 or Ah1, in which
Aa1 is 1,1′-biphenyl-3-yl, or 1,1′-biphenyl-4-yl,
such as, for example,
   3′-(R61)-1,1′-biphenyl-3-yl, 4′-(R61)-1,1′-biphenyl-3-yl, 3′-(R61)-1,1′-biphenyl-4-yl or 4′-(R61)-1,1′-biphenyl-4-yl,
Ah1 is phenyl-thiophenyl, or phenyl-pyridinyl,
such as, for example,
   [3-(R61)-phenyl]-thiophenyl, [4-(R61)-phenyl]-thiophenyl, [3-(R61)-phenyl]-pyridinyl or [4-(R61)-phenyl]-pyridinyl,
   e.g. 5-[3-(R61)-phenyl]-thiophen-2-yl, 5-[4-(R61)-phenyl]-thiophen-2-yl, 2-[3-(R61)-phenyl]-pyridin-4-yl, 2-[4-(R61)-phenyl]-pyridin-4-yl, 6-[3-(R61)-phenyl]-pyridin-3-yl or 6-[4-(R61)-phenyl]-pyridin-3-yl,
in which
R61 is methoxy, hydroxyl, trifluoromethyl, hydroxymethyl, methylsulphonylamino, methylcarbonylamino, dimethylaminosulphonyl, -T2-N(R611)R612, —U-T3-N(R613)R614, -T4-Het3, or —V-T5-Het4, in which T2 is a bond, methylene, dimethylene or trimethylene,
R611 is hydrogen, methyl, cyclopropyl, cyclopentyl, 2-methoxyethyl, acetyl or methylsulphonyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4-methyl-piperazino,
U is —O— (oxygen) or —C(O)NH—,
T3 is dimethylene or trimethylene,
R613 and R614 are methyl,
or R613 and R614 together and with inclusion of the nitrogen atom, to which they are bonded, form a heterocyclic ring Het2, in which Het2 is morpholino, piperidino, pyrrolidino or 4-methyl-piperazino,
T4 is a bond, methylene, dimethylene or trimethylene,
Het3 is 1-methyl-piperidinyl or 1-methyl-pyrrolidinyl,
V is —O— (oxygen) or —C(O)NH—,
T5 is a bond, methylene, dimethylene or trimethylene,
Het4 is 1-methyl-piperidinyl or 1-methyl-pyrrolidinyl;
or
Q1 is substituted by R61 on the terminal ring, and is Hh1 or Ha1, in which
Hh1 is pyridinyl-thiophenyl, or bipyridyl,
such as, for example,
   [2-(R61)-pyridin-4-yl]-thiophenyl or [6-(R61)-pyridin-3-yl]-thiophenyl, e.g. 5-[2-(R61)-pyridin-4-yl]-thiophen-2-yl or 5-[6-(R61)-pyridin-3-yl]-thiophen-2-yl, or
   [2-(R61)-pyridin-4-yl]-pyridinyl or [6-(R61)-pyridin-3-yl]-pyridinyl, e.g. 2-[2-(R61)-pyridin-4-yl]-pyridin-4-yl, 2-[6-(R61)-pyridin-3-yl]-pyridin-4-yl, 6-[2-(R61)-pyridin-4-yl]-pyridin-3-yl or 6-[6-(R61)-pyridin-3-yl]-pyridin-3-yl,
Ha1 is 3-(pyridinyl)-phenyl, or 4-(pyridinyl)-phenyl,
such as, for example,
   3-[2-(R61)-pyridin-4-yl]-phenyl, 3-[6-(R61)-pyridin-3-yl]-phenyl, 4-[2-(R61)-pyridin-4-yl]-phenyl or 4-[6-(R61)-pyridin-3-yl]-phenyl,
in which
R61 is methoxy, or -T2-N(R611)R612, in which
T2 is a bond,
R611 and R612 are independently hydrogen or methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino;
or
Q1 is 3-(1-methyl-pyrazolyl)-phenyl, 4-(1-methyl-pyrazolyl)-phenyl, 3-(methyl-thiazolyl)-phenyl, 4-(methyl-thiazolyl)-phenyl, 3-(dimethyl-isoxazolyl)-phenyl, 4-(dimethyl-isoxazolyl)-phenyl, (1-methyl-pyrazolyl)-thiophenyl, (1-methyl-pyrazolyl)-pyridinyl, (methyl-thiazolyl)-thiophenyl, (methyl-thiazolyl)-pyridinyl, 3-(benzo[1,3]dioxolyl)-phenyl, 4-(benzo[1,3]dioxolyl)-phenyl, 3-(2,3-dihydrobenzofuranyl)-phenyl, 4-(2,3-dihydrobenzofuranyl)-phenyl, 3-(1-methyl-indolyl)-phenyl, or 4-(1-methyl-indolyl)-phenyl,
such as, for example,
   3-(1-methyl-pyrazol-4-yl)-phenyl, 4-(1-methyl-pyrazol-4-yl)-phenyl, 3-(2-methyl-thiazol-4-yl)-phenyl, 4-(2-methyl-thiazol-4-yl)-phenyl, 3-(3,5-dimethyl-isoxazol-4-yl)-phenyl, 4-(3,5-dimethyl-isoxazol-4-yl)-phenyl, (1-methyl-pyrazol-4-yl)-thiophenyl e.g. 5-(1-methyl-pyrazol-4-yl)-thiophen-2-yl, (1-methyl-pyrazol-4-yl)-pyridinyl e.g. 6-(1-methyl-pyrazol-4-yl)-pyridin-3-yl or 2-(1-methyl-pyrazol-4-yl)-pyridin-4-yl, (2-methyl-thiazol-4-yl)-thiophenyl e.g. 5-(2-methyl-thiazol-4-yl)-thiophen-2-yl, (2-methyl-thiazol-4-yl)-pyridinyl e.g. 6-(2-methyl-thiazol-4-yl)-pyridin-3-yl or 2-(2-methyl-thiazol-4-yl)-pyridin-4-yl, 3-(benzo[1,3]dioxol-5-yl)-phenyl, 4-(benzo[1,3]dioxol-5-yl)-phenyl, 3-(2,3-dihydrobenzofuran-5-yl)-phenyl, 4-(2,3-dihydrobenzofuran-5-yl)-phenyl, 3-(1-methyl-indol-5-yl)-phenyl or 4-(1-methyl-indol-5-yl)-phenyl;
or
Q1 is 3-[1N—(R61)-pyrazolyl]-phenyl, 4-[1N—(R61)-pyrazolyl]-phenyl, [1N—(R61)-pyrazolyl)-thiophenyl, [1N—(R61)-pyrazolyl)-pyridinyl, 3-[1N—(R61)-triazolyl]-phenyl, or 4-[1N—(R61)-triazoly]-phenyl,
such as, for example,
   3-[1N—(R61)-pyrazol-4-yl]-phenyl, 4-[1N—(R61)-pyrazol-4-yl]-phenyl, [1N—(R61)-pyrazol-4-yl)-thiophenyl e.g. 5-[1N—(R61)-pyrazol-4-yl)-thiophen-2-yl, [1N—(R61)-pyrazol-4-yl)-pyridinyl e.g. 2-[1N—(R61)-pyrazol-4-yl)-pyridin-4-yl or 6-[1N—(R61)-pyrazol-4-yl)-pyridin-3-yl, 3-[1N—(R61)-triazol-4-yl]-phenyl or 4-[1N—(R61)-triazol-4-yl]-phenyl,
in which
R61 is -T2-N(R611)R612, or -T4-Het3, in which
T2 is dimethylene or trimethylene,
R611 is hydrogen, methyl, cyclopropyl, cyclopentyl, 2-methoxyethyl, acetyl or methylsulphonyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4-methyl-piperazino,
T4 is a bond, methylene, dimethylene or trimethylene,
Het3 is 1-methyl-piperidinyl or 1-methyl-pyrrolidinyl;
R7 is hydroxyl;
and the salts of these compounds.

Other compounds according to aspect A of the present invention to be emphasized are those compounds of formula I in which
R1, R2, R3, R4 and R5 are hydrogen,
R6 is-T1-Q1, in which
T1 is a bond;
either
Q1 is substituted by R61 on the terminal ring, and is Aa1 or Ah1, in which
Aa1 is 1,1′-biphenyl-3-yl, or 1,1′-biphenyl-4-yl,
such as, for example,
   3′-(R61)-1,1′-biphenyl-3-yl, 4′-(R61)-1,1′-biphenyl-3-yl, 3′-(R61)-1,1′-biphenyl-4-yl or 4′-(R61)-1,1′-biphenyl-4-yl,
Ah1 is phenyl-thiophenyl, or phenyl-pyridinyl,
such as, for example,
   [3-(R61)-phenyl]-thiophenyl, [4-(R61)-phenyl]-thiophenyl, [3-(R61)-phenyl]-pyridinyl or [4-(R61)-phenyl]-pyridinyl,
   e.g. 5-[3-(R61)-phenyl]-thiophen-2-yl, 5-[4-(R61)-phenyl]-thiophen-2-yl, 2-[3-(R61)-phenyl]-pyridin-4-yl, 2-[4-(R61)-phenyl]-pyridin-4-yl, 6-[3-(R61)-phenyl]-pyridin-3-yl or 6-[4-(R61)-phenyl]-pyridin-3-yl,
in which
R61 is methoxy, hydroxyl, trifluoromethyl, hydroxymethyl, methylsulphonylamino, methylcarbonylamino, dimethylaminosulphonyl, -T2-N(R611)R612, —U-T3-N(R613)R614, -T4-Het3, or —V-T5-Het4, in which
T2 is a bond, methylene, dimethylene or trimethylene,
R611 is hydrogen, methyl, cyclopropyl, cyclopentyl, 2-methoxyethyl, acetyl or methylsulphonyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4-methyl-piperazino,
U is —O— (oxygen) or —C(O)NH—,
T3 is dimethylene or trimethylene,
R613 is methyl,
R614 is methyl,
or R613 and R614 together and with inclusion of the nitrogen atom, to which they are bonded, form a heterocyclic ring Het2, in which
Het2 is morpholino, piperidino, pyrrolidino or 4-methyl-piperazino,
T4 is a bond, methylene, dimethylene or trimethylene,
Het3 is 1-methyl-piperidinyl or 1-methyl-pyrrolidinyl,
V is —O— (oxygen) or —C(O)NH—,
T5 is a bond, methylene, dimethylene or trimethylene,
Het4 is 1-methyl-piperidinyl or 1-methyl-pyrrolidinyl;
or
Q1 is substituted by R61 on the terminal ring, and is Hh1 or Ha1, in which
Hh1 is pyridinyl-thiophenyl, or bipyridyl,
such as, for example,
   [2-(R61)-pyridin-4-yl]-thiophenyl or [6-(R61)-pyridin-3-yl]-thiophenyl, e.g. 5-[2-(R61)-pyridin-4-yl]-thiophen-2-yl or 5-[6-(R61)-pyridin-3-yl]-thiophen-2-yl, or
   [2-(R61)-pyridin-4-yl]-pyridinyl or [6-(R61)-pyridin-3-yl]-pyridinyl, e.g. 2-[2-(R61)-pyridin-4-yl]-pyridin-4-yl, 2-[6-(R61)-pyridin-3-yl]-pyridin-4-yl, 6-[2-(R61)-pyridin-4-yl]-pyridin-3-yl or 6-[6-(R61)-pyridin-3-yl]-pyridin-3-yl,
Ha1 is 3-(pyridinyl)-phenyl, or 4-(pyridinyl)-phenyl,
such as, for example,
   3-[2-(R61)-pyridin-4-yl]-phenyl, 3-[6-(R61)-pyridin-3-yl]-phenyl, 4-[2-(R61)-pyridin-4-yl]-phenyl or 4-[6-(R61)-pyridin-3-yl]-phenyl,
in which
R61 is methoxy, or -T2-N(R611)R612, in which
T2 is a bond,
R611 is hydrogen or methyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino;
or
Q1 is 3-(1-methyl-pyrazolyl)-phenyl, 4-(1-methyl-pyrazolyl)-phenyl, 3-(methyl-thiazolyl)-phenyl, 4-(methyl-thiazolyl)-phenyl, 3-(dimethyl-isoxazolyl)-phenyl, 4-(dimethyl-isoxazolyl)-phenyl,
   (1-methyl-pyrazolyl)-thiophenyl, (1-methyl-pyrazolyl)-pyridinyl, (methyl-thiazolyl)-thiophenyl,
   (methyl-thiazolyl)-pyridinyl, 3-(benzo[1,3]dioxolyl)-phenyl, 4-(benzo[1,3]dioxolyl)-phenyl, 3-(2,3-dihydrobenzofuranyl)-phenyl, 4-(2,3-dihydrobenzofuranyl)-phenyl, 3-(1-methyl-indolyl)-phenyl, or 4-(1-methyl-indolyl)-phenyl,
such as, for example,
   3-(1-methyl-pyrazol-4-yl)-phenyl, 4-(1-methyl-pyrazol-4-yl)-phenyl, 3-(2-methyl-thiazol-4-yl)-phenyl, 4-(2-methyl-thiazol-4-yl)-phenyl, 3-(3,5-dimethyl-isoxazol-4-yl)-phenyl, 4-(3,5-dimethyl-isoxazol-4-yl)-phenyl, (1-methyl-pyrazol-4-yl)-thiophenyl e.g. 5-(1-methyl-pyrazol-4-yl)-thiophen-2-yl, (1-methyl-pyrazol-4-yl)-pyridinyl e.g. 6-(1-methyl-pyrazol-4-yl)-pyridin-3-yl or 2-(1-methyl-pyrazol-4-yl)-pyridin-4-yl, (2-methyl-thiazol-4-yl)-thiophenyl e.g. 5-(2-methyl-thiazol-4-yl)-thiophen-2-yl, (2-methyl-thiazol-4-yl)-pyridinyl e.g. 6-(2-methyl-thiazol-4-yl)-pyridin-3-yl or 2-(2-methyl-thiazol-4-yl)-pyridin-4-yl, 3-(benzo[1,3]dioxol-5-yl)-phenyl, 4-(benzo[1,3]dioxol-5-yl)-phenyl, 3-(2,3-dihydrobenzofuran-5-yl)-phenyl, 4-(2,3-dihydrobenzofuran-5-yl)-phenyl, 3-(1-methyl-indol-5-yl)-phenyl or 4-(1-methyl-indol-5-yl)-phenyl;
or
Q1 is 3-[1N—(R61)-pyrazolyl]-phenyl, 4-[1N—(R61)-pyrazolyl]-phenyl, [1N—(R61)-pyrazolyl)-thiophenyl, [1N—(R61)-pyrazolyl)-pyridinyl, 3-[1N—(R61)-triazolyl]-phenyl, or 4-[1N—(R61)-triazolyl]-phenyl,
such as, for example,
   3-[1N—(R61)-pyrazol-4-yl]-phenyl, 4-[1N—(R61)-pyrazol-4-yl]-phenyl, [1N—(R61)-pyrazol-4-yl)-thiophenyl e.g. 5-[1N—(R61)-pyrazol-4-yl)-thiophen-2-yl, [1N—(R61)-pyrazol-4-yl)-pyridinyl e.g. 2-[1N—(R61)-pyrazol-4-yl)-pyridin-4-yl or 6-[1N—(R61)-pyrazol-4-yl)-pyridin-3-yl,
   3-[1N—(R61)-triazol-4-yl]-phenyl or 4-[1N—(R61)-triazol-4-yl]-phenyl,
in which
R61 is -T2-N(R611)R612, or -T4-Het3, in which
T2 is dimethylene or trimethylene,
R611 is hydrogen, methyl, cyclopropyl, cyclopentyl, 2-methoxyethyl, acetyl or methylsulphonyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4-methyl-piperazino,
T4 is a bond, methylene, dimethylene or trimethylene,
Het3 is 1-methyl-piperidinyl or 1-methyl-pyrrolidinyl;
R7 is 2-aminophenyl;
and the salts of these compounds.

Compounds according to aspect A of the present invention to be more emphasized are those compounds of formula I in which
R1, R2, R3, R4 and R5 are hydrogen,
R6 is -T1-Q1, in which T1 is a bond;
either
Q1 is substituted by R61 on the terminal ring, and is Aa1 or Ah1, in which
Aa1 is 1,1′-biphenyl-3-yl, or 1,1′-biphenyl-4-yl,
such as, for example,
   3′-(R61)-1,1′-biphenyl-3-yl, 4′-(R61)-1,1′-biphenyl-3-yl, 3′-(R61)-1,1′-biphenyl-4-yl or 4′-(R61)-1,1′-biphenyl-4-yl,
Ah1 is phenyl-thiophenyl, or phenyl-pyridinyl,
such as, for example,
   [3-(R61)-phenyl]-thiophenyl, [4-(R61)-phenyl]-thiophenyl, [3-(R61)-phenyl]-pyridinyl or [4-(R61)-phenyl]-pyridinyl,
   e.g. 5-[3-(R61)-phenyl]-thiophen-2-yl, 5-[4-(R61)-phenyl]-thiophen-2-yl, 2-[3-(R61)-phenyl]-pyridin-4-yl, 2-[4-(R61)-phenyl]-pyridin-4-yl, 6-[3-(R61)-phenyl]-pyridin-3-yl or 6-[4-(R61)-phenyl]-pyridin-3-yl,
in which
R61 is any one selected from 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl;
or
Q1 is substituted by R61 on the terminal ring, and is Hh1 or Ha1, in which
Hh1 is pyridinyl-thiophenyl, or bipyridyl,
such as, for example,
   [2-(R61)-pyridin-4-yl]-thiophenyl or [6-(R61)-pyridin-3-yl]-thiophenyl, e.g. 5-[2-(R61)-pyridin-4-yl]-thiophen-2-yl or 5-[6-(R61)-pyridin-3-yl]-thiophen-2-yl, or
   [2-(R61)-pyridin-4-yl]-pyridinyl or [6-(R61)-pyridin-3-yl]-pyridinyl, e.g. 2-[2-(R61)-pyridin-4-yl]-pyridin-4-yl, 2-[6-(R61)-pyridin-3-yl]-pyridin-4-yl, 6-[2-(R61)-pyridin-4-yl]-pyridin-3-yl or 6-[6-(R61)-pyridin-3-yl]-pyridin-3-yl,
Ha1 is 3-(pyridinyl)-phenyl, or 4-(pyridinyl)-phenyl,
such as, for example,
   3-[2-(R61)-pyridin-4-yl]-phenyl, 3-[6-(R61)-pyridin-3-yl]-phenyl, 4-[2-(R61)-pyridin-4-yl]-phenyl or 4-[6-(R61)-pyridin-3-yl]-phenyl,
in which
R61 is any one selected from methylsulphonylamino, acetamido, amino, dimethylamino, morpholino, piperidino, pyrrolidino, 4-methyl-piperazino, hydroxy, trifluoromethyl and methoxy;
or
Q1 is 3-(1-methyl-pyrazol-4-yl)-phenyl, 4-(1-methyl-pyrazol-4-yl)-phenyl, 3-(2-methyl-thiazol-4-yl)-phenyl, 4-(2-methyl-thiazol-4-yl)-phenyl, 3-(3,5-dimethyl-isoxazol-4-yl)-phenyl, 4-(3,5-dimethyl-isoxazol-4-yl)-phenyl, (1-methyl-pyrazol-4-yl)-thiophenyl e.g. 5-(1-methyl-pyrazol-4-yl)-thiophen-2-yl, (1-methyl-pyrazol-4-yl)-pyridinyl e.g. 6-(1-methyl-pyrazol-4-yl)-pyridin-3-yl or 2-(1-methyl-pyrazol-4-yl)-pyridin-4-yl, (2-methyl-thiazol-4-yl)-thiophenyl e.g. 5-(2-methyl-thiazol-4-yl)-thiophen-2-yl, (2-methyl-thiazol-4-yl)-pyridinyl e.g. 6-(2-methyl-thiazol-4-yl)-pyridin-3-yl or 2-(2-methyl-thiazol-4-yl)-pyridin-4-yl, 3-(benzo[1,3]dioxol-5-yl)-phenyl, 4-(benzo[1,3]dioxol-5-yl)-phenyl, 3-(2,3-dihydrobenzofuran-5-yl)-phenyl, 4-(2,3-dihydrobenzofuran-5-yl)-phenyl, 3-(1-methyl-indol-5-yl)-phenyl, or 4-(1-methyl-indol-5-yl)-phenyl;
or
Q1 is 3-[1N—(R61)-pyrazol-4-yl]-phenyl, 4-[1N—(R61)-pyrazol-4-yl]-phenyl, [1N—(R61)-pyrazol-4-yl)-thiophenyl e.g. 5-[1N—(R61)-pyrazol-4-yl)-thiophen-2-yl, [1N—(R61)-pyrazol-4-yl)-pyridinyl e.g. 2-[1N—(R61)-pyrazol-4-yl)-pyridin-4-yl or 6-[1N—(R61)-pyrazol-4-yl)-pyridin-3-yl,
   3-[1N—(R61)-triazol-4-yl]-phenyl, or 4-[1N—(R61)-triazol-4-yl]-phenyl,
in which
R61 is any one selected from 3-morpholin-4-yl-propyl, 2-morpholin-4-yl-ethyl, 3-(4-methyl-piperazin-1-yl)-propyl, 2-(4-methyl-piperazin-1-yl)-ethyl, 3-pyrrolidin-1-yl-propyl, 2-pyrrolidin-1-yl-ethyl, 3-piperidin-1-yl-propyl, 2-piperidin-1-yl-ethyl, 2-dimethylamino-ethyl and 3-dimethylamino-propyl;
R7 is hydroxyl;
and the salts of these compounds.

Compounds according to aspect A of the present invention to be more emphasized are those compounds of formula I in which
R1, R2, R3, R4 and R5 are hydrogen,
R6 is -T1-Q1, in which T1 is a bond;
either
Q1 is substituted by R61 on the terminal ring, and is Aa1 or Ah1, in which
Aa1 is 1,1′-biphenyl-3-yl, or 1,1′-biphenyl-4-yl,
such as, for example,
   3′-(R61)-1,1′-biphenyl-3-yl, 4′-(R61)-1,1′-biphenyl-3-yl, 3′-(R61)-1,1′-biphenyl-4-yl or 4′-(R61)-1,1′-biphenyl-4-yl,
Ah1 is phenyl-thiophenyl, or phenyl-pyridinyl,
such as, for example,
   [3-(R61)-phenyl]-thiophenyl, [4-(R61)-phenyl]-thiophenyl, [3-(R61)-phenyl]-pyridinyl or [4-(R61)-phenyl]-pyridinyl,
   e.g. 5-[3-(R61)-phenyl]-thiophen-2-yl, 5-[4-(R61)-phenyl]-thiophen-2-yl, 2-[3-(R61)-phenyl]-pyridin-4-yl, 2-[4-(R61)-phenyl]-pyridin-4-yl, 6-[3-(R61)-phenyl]-pyridin-3-yl or 6-[4-(R61)-phenyl]-pyridin-3-yl,
in which
R61 is any one selected from 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl;
or
Q1 is substituted by R61 on the terminal ring, and is Hh1 or Ha1, in which
Hh1 is pyridinyl-thiophenyl, or bipyridyl,
such as, for example,
   [2-(R61)-pyridin-4-yl]-thiophenyl or [6-(R61)-pyridin-3-yl]-thiophenyl, e.g. 5-[2-(R61)-pyridin-4-yl]-thiophen-2-yl or 5-[6-(R61)-pyridin-3-yl]-thiophen-2-yl, or
   [2-(R61)-pyridin-4-yl]-pyridinyl or [6-(R61)-pyridin-3-yl]-pyridinyl, e.g. 2-[2-(R61)-pyridin-4-yl]-pyridin-4-yl, 2-[6-(R61)-pyridin-3-yl]-pyridin-4-yl, 6-[2-(R61)-pyridin-4-yl]-pyridin-3-yl or 6-[6-(R61)-pyridin-3-yl]-pyridin-3-yl,
Ha1 is 3-(pyridinyl)-phenyl, or 4-(pyridinyl)-phenyl,
such as, for example,
   3-[2-(R61)-pyridin-4-yl]-phenyl, 3-[6-(R61)-pyridin-3-yl]-phenyl, 4-[2-(R61)-pyridin-4-yl]-phenyl or 4-[6-(R61)-pyridin-3-yl]-phenyl,
in which
R61 is any one selected from methylsulphonylamino, acetamido, amino, dimethylamino, morpholino, piperidino, pyrrolidino, 4-methyl-piperazino, hydroxy, trifluoromethyl and methoxy;
or
Q1 is 3-(1-methyl-pyrazol-4-yl)-phenyl, 4-(1-methyl-pyrazol-4-yl)-phenyl, 3-(2-methyl-thiazol-4-yl)-phenyl, 4-(2-methyl-thiazol-4-yl)-phenyl, 3-(3,5-dimethyl-isoxazol-4-yl)-phenyl, 4-(3,5-dimethyl-isoxazol-4-yl)-phenyl, (1-methyl-pyrazol-4-yl)-thiophenyl e.g. 5-(1-methyl-pyrazol-4-yl)-thiophen-2-yl, (1-methyl-pyrazol-4-yl)-pyridinyl e.g. 6-(1-methyl-pyrazol-4-yl)-pyridin-3-yl or 2-(1-methyl-pyrazol-4-yl)-pyridin-4-yl, (2-methyl-thiazol-4-yl)-thiophenyl e.g. 5-(2-methyl-thiazol-4-yl)-thiophen-2-yl, (2-methyl-thiazol-4-yl)-pyridinyl e.g. 6-(2-methyl-thiazol-4-yl)-pyridin-3-yl or 2-(2-methyl-thiazol-4-yl)-pyridin-4-yl, 3-(benzo[1,3]dioxol-5-yl)-phenyl, 4-(benzo[1,3]dioxol-5-yl)-phenyl, 3-(2,3-dihydrobenzofuran-5-yl)-phenyl, 4-(2,3-dihydrobenzofuran-5-yl)-phenyl, 3-(1-methyl-indol-5-yl)-phenyl, or 4-(1-methyl-indol-5-yl)-phenyl;
or
Q1 is 3-[1N—(R61)-pyrazol-4-yl]-phenyl, 4-[1N—(R61)-pyrazol-4-yl]-phenyl, [1N—(R61)-pyrazol-4-yl)-thiophenyl e.g. 5-[1N—(R61)-pyrazol-4-yl)-thiophen-2-yl, [1N—(R61)-pyrazol-4-yl)-pyridinyl e.g. 2-[1N—(R61)-pyrazol-4-yl)-pyridin-4-yl or 6-[1N—(R61)-pyrazol-4-yl)-pyridin-3-yl, 3-[1N—(R61)-triazol-4-yl]-phenyl, or 4-[1N—(R61)-triazol-4-yl]-phenyl,
in which
R61 is any one selected from 3-morpholin-4-yl-propyl, 2-morpholin-4-yl-ethyl, 3-(4-methyl-piperazin-1-yl)-propyl, 2-(4-methyl-piperazin-1-yl)-ethyl, 3-pyrrolidin-1-yl-propyl, 2-pyrrolidin-1-yl-ethyl, 3-piperidin-1-yl-propyl, 2-piperidin-1-yl-ethyl, 2-dimethylamino-ethyl and 3-dimethylamino-propyl;
R7 is 2-aminophenyl;
and the salts of these compounds.

Compounds according to aspect A of the present invention to be more emphasized are those compounds of formula I in which

R1, R2, R3, R4 and R5 are hydrogen,

R6 is -T1-Q1, in which T1 is a bond;

Q1 is any one selected from the group consisting of

3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 3′-(morpholin-4-yl-methyl)-biphenyl-4-yl, 4′-(morpholin-4-yl-methyl)-biphenyl-4-yl, 4′-(3-morpholin-4-yl-propyl)-biphenyl-3-yl, 4′-(3-morpholin-4-yl-propyl)-biphenyl-4-yl, 3′-(3-morpholin-4-yl-propyl)-biphenyl-3-yl, 3′-(3-morpholin-4-yl-propyl)-biphenyl-4-yl, 4′-(4-methyl-piperazin-1-ylmethyl)-biphenyl-3-yl, 4′-(4-methyl-piperazin-1-ylmethyl)-biphenyl-4-yl, 3′-(4-methyl-piperazin-1-ylmethyl)-biphenyl-3-yl, 3′-(4-methyl-piperazin-1-ylmethyl)-biphenyl-4-yl, 4′-(2-morpholin-4-yl-ethoxy)-biphenyl-3-yl, 4′-(2-morpholin-4-yl-ethoxy)-biphenyl-4-yl, 3′-(2-morpholin-4-yl-ethoxy)-biphenyl-3-yl, 3′-(2-morpholin-4-yl-ethoxy)-biphenyl-4-yl, 4′-(3-morpholin-4-yl-propoxy)-biphenyl-3-yl, 4′-(3-morpholin-4-yl-propoxy)-biphenyl-4-yl, 3′-(3-morpholin-4-yl-propoxy)-biphenyl-3-yl, 3′-(3-morpholin-4-yl-propoxy)-biphenyl-4-yl, 4′-[2-(4-methyl-piperazin-1-yl)-ethoxy]-biphenyl-3-yl, 4′-[2-(4-methyl-piperazin-1-yl)-ethoxy]-biphenyl-4-yl, 3′-[2-(4-methyl-piperazin-1-yl)-ethoxy]-biphenyl-3-yl, 3′-[2-(4-methyl-piperazin-1-yl)-ethoxy]-biphenyl-4-yl, 4′-(2-pyrrolidin-1-yl-ethoxy]-biphenyl-3-yl, 4′-(2-pyrrolidin-1-yl-ethoxy]-biphenyl-4-yl, 3′-(2-pyrrolidin-1-yl-ethoxy]-biphenyl-3-yl, 3′-(2-pyrrolidin-1-yl-ethoxy]-biphenyl-4-yl, 3′-(3-pyrrolidin-1-yl-propoxy]-biphenyl-4-yl, 4′-(3-pyrrolidin-1-yl-propoxy]-biphenyl-4-yl, 3′-(3-pyrrolidin-1-yl-propoxy]-biphenyl-3-yl, 4′-(3-pyrrolidin-1-yl-propoxy]-biphenyl-3-yl, 4′-[3-(4-methyl-piperazin-1-yl)-propoxy]-biphenyl-4-yl, 3′-[3-(4-methyl-piperazin-1-yl)-propoxy]-biphenyl-4-yl, 4′-[3-(4-methyl-piperazin-1-yl)-propoxy]-biphenyl-3-yl, 3′-[3-(4-methyl-piperazin-1-yl)-propoxy]-biphenyl-3-yl,

4′-(2-(1-methyl-piperidin-4-yl)-ethoxy)-biphenyl-4-yl, 4′-(2-(1-methyl-piperidin-4-yl)-ethoxy)-biphenyl-3-yl,

3′-(2-(1-methyl-piperidin-4-yl)-ethoxy)-biphenyl-4-yl, 3′-(2-(1-methyl-piperidin-4-yl)-ethoxy)-biphenyl-3-yl,

2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 3′-dimethylaminomethyl-biphenyl-4-yl, 3′-dimethylaminomethyl-biphenyl-3-yl, 3′-[(2-dimethylamino-ethylamino)-carbonyl]-biphenyl-4-yl, 4′-[(2-dimethylamino-ethylamino)-carbonyl]-biphenyl-4-yl, 4′-[(2-dimethylamino-ethylamino)-carbonyl]-biphenyl-3-yl, 3′-[(2-dimethylamino-ethylamino)-carbonyl]-biphenyl-3-yl, 2′-methylsulphonylamino-biphenyl-4-yl, 3′-methylsulphonylamino-biphenyl-4-yl, 4′-methylsulphonylamino-biphenyl-4-yl, 2′-methylsulphonylamino-biphenyl-3-yl, 3′-methylsulphonylamino-biphenyl-3-yl, 4′-methylsulphonylamino-biphenyl-3-yl, 4′-methylsulphonylamino-biphenyl-3-yl, 4′-dimethylsulphamoyl-biphenyl-4-yl, 4′-dimethylsulphamoyl-biphenyl-3-yl, 3′-dimethylsulphamoyl-biphenyl-4-yl, 3′-dimethylsulphamoyl-biphenyl-3-yl, 3′-acetamido-biphenyl-4-yl, 4′-acetamido-biphenyl-4-yl, 3′-acetamido-biphenyl-3-yl, 4′-acetamido-biphenyl-3-yl, 3′-amino-biphenyl-4-yl, 3′-dimethylamino-biphenyl-4-yl, 4′-morpholin-4-yl-biphenyl-4-yl, 4′-hydroxy-biphenyl-4-yl, 3′-trifluoromethyl-biphenyl-4-yl, 4′-methoxy-biphenyl-4-yl, 3′-amino-biphenyl-3-yl, 3′-dimethylamino-biphenyl-3-yl, 4′-morpholin-4-yl-biphenyl-3-yl, 4′-hydroxy-biphenyl-3-yl, 3′-trifluoromethyl-biphenyl-3-yl, 4′-methoxy-biphenyl-3-yl, 4′-amino-biphenyl-4-yl, 4′-dimethylamino-biphenyl-4-yl, 3′-morpholin-4-yl-biphenyl-4-yl, 3′-hydroxy-biphenyl-4-yl, 4′-trifluoromethyl-biphenyl-4-yl, 3′-methoxy-biphenyl-4-yl, 4′-amino-biphenyl-3-yl, 4′-dimethylamino-biphenyl-3-yl, 3′-morpholin-4-yl-biphenyl-3-yl, 3′-hydroxy-biphenyl-3-yl, 4′-trifluoromethyl-biphenyl-3-yl and 3′-methoxy-biphenyl-3-yl,

4′-(2-methoxy-ethylamino)methyl-biphenyl-3-yl, 4′-(2-methoxy-ethylamino)methyl-biphenyl-4-yl, 3′-(2-methoxy-ethylamino)methyl-biphenyl-3-yl, 3′-(2-methoxy-ethylamino)methyl-biphenyl-4-yl, 4′-aminomethyl-biphenyl-3-yl, 4′-aminomethyl-biphenyl-4-yl, 3′-aminomethyl-biphenyl-3-yl, 3′-aminomethyl-biphenyl-4-yl, 4′-(acetylamino)-methyl-biphenyl-4-yl, 4′-(methylsulphonylamino)-methyl-biphenyl-4-yl, 3′-(acetylamino)-methyl-biphenyl-3-yl, 3′-(methylsulphonylamino)-methyl-biphenyl-3-yl, 4′-(acetylamino)-methyl-biphenyl-3-yl, 4′-(methylsulphonylamino)-methyl-biphenyl-3-yl, 3′-(acetylamino)-methyl-biphenyl-4-yl, 3′-(methylsulphonylamino)-methyl-biphenyl-4-yl, 4′-cyclopentylaminomethyl-biphenyl-4-yl, 4′-cyclopentylaminomethyl-biphenyl-3-yl, 3′-cyclopentylaminomethyl-biphenyl-4-yl, 3′-cyclopentylaminomethyl-biphenyl-3-yl, 4′-cyclopropylaminomethyl-biphenyl-3-yl, 4′-cyclopropylaminomethyl-biphenyl-4-yl, 3′-cyclopropylaminomethyl-biphenyl-3-yl, 3′-cyclopropylaminomethyl-biphenyl-4-yl, 3′-hydroxymethyl-biphenyl-4-yl, 3′-hydroxymethyl-biphenyl-3-yl,

4′-hydroxymethyl-biphenyl-4-yl, 4′-hydroxymethyl-biphenyl-3-yl, 5-[2-(4-methyl-piperazin-1-yl)-pyridin-4-yl]-thiophen-2-yl, 5-(1-methyl-pyrazol-4-yl)-thiophen-2-yl, 6-(1-methyl-pyrazol-4-yl)-pyridin-3-yl,

2′-(4-methyl-piperazin-1-yl)-2,4′-bipyridyl-5-yl, 5-(2-methyl-thiazol-4-yl)-thiophen-2-yl, 5-[4-(2-morpholin-4-yl-ethyl)-phenyl]-thiophen-2-yl, 5-[3-(2-morpholin-4-yl-ethyl)-phenyl]-thiophen-2-yl, 5-[4-(morpholin-4-yl-methyl)-phenyl]-thiophen-2-yl, 5-[3-(morpholin-4-yl-methyl)-phenyl]-thiophen-2-yl,

5-[4-(2-morpholin-4-yl-ethoxy)-phenyl]-thiophen-2-yl, 5-[3-(2-morpholin-4-yl-ethoxy)-phenyl]-thiophen-2-yl, 5-[4-(3-morpholin-4-yl-propoxy)-phenyl]-thiophen-2-yl, 5-[3-(3-morpholin-4-yl-propoxy)-phenyl]-thiophen-2-yl, 5-{4-[2-(4-methyl-piperazin-1-yl)-ethoxy]-phenyl}-thiophen-2-yl, 5-{3-[2-(4-methyl-piperazin-1-yl)-ethoxy]-phenyl}-thiophen-2-yl, 5-[4-(2-pyrrolidin-1-yl-ethoxy)-phenyl]-thiophen-2-yl, 5-[3-(2-pyrrolidin-1-yl-ethoxy)-phenyl]-thiophen-2-yl, 5-(4-dimethylaminomethyl-phenyl)-thiophen-2-yl, 5-(3-dimethylaminomethyl-phenyl)-thiophen-2-yl, 6-(4-dimethylaminomethyl-phenyl)-pyridin-3-yl, 6-(3-dimethylaminomethyl-phenyl)-pyridin-3-yl, 6-[4-(2-pyrrolidin-1-yl-ethoxy)-phenyl]-pyridin-3-yl, 6-[3-(2-pyrrolidin-1-yl-ethoxy)-phenyl]-pyridin-3-yl, 5-(3-aminomethyl-phenyl)-thiophen-2-yl, 5-[3-(acetylamino)-methyl-phenyl]-thiophen-2-yl, 5-[3-(methylsulphonylamino)-methyl-phenyl]-thiophen-2-yl, 5-(4-dimethylsulphamoyl-phenyl)-thiophen-2-yl, 5-(4-aminomethyl-phenyl)-thiophen-2-yl, 5-[4-(acetylamino)-methyl-phenyl]-thiophen-2-yl, 5-[4-(methylsulphonylamino)-methyl-phenyl]-thiophen-2-yl, 5-(3-dimethylsulphamoyl-phenyl)-thiophen-2-yl, 4-[2-(4-methyl-piperazin-1-yl)-pyridin-4-yl]-phenyl, 3-[2-(4-methyl-piperazin-1-yl)-pyridin-4-yl]-phenyl, 4-(6-amino-pyridin-3-yl)-phenyl, 3-(6-amino-pyridin-3-yl)-phenyl, 4-(6-methoxy-pyridin-3-yl)-phenyl, 3-(6-methoxy-pyridin-3-yl)-phenyl, 3-(1-methyl-pyrazol-4-yl)-phenyl, 4-(1-methyl-pyrazol-4-yl)-phenyl, 4-(3,5-dimethyl-isoxazol-4-yl)-phenyl, 3-(3,5-dimethyl-isoxazol-4-yl)-phenyl, 4-(1-methyl-indol-5-yl)-phenyl, 3-(1-methyl-indol-5-yl)-phenyl,

4-{1-(2-morpholin-4-yl-ethyl)-[1,2,3]triazol-4-yl}-phenyl, 4-{1-(2-piperidin-1-yl-ethyl)-[1,2,3]triazol-4-yl}-phenyl, 3-{1-(2-morpholin-4-yl-ethyl)-[1,2,3]triazol-4-yl}-phenyl, 3-{1-(2-piperidin-1-yl-ethyl)-[1,2,3]triazol-4-yl}-phenyl, 4-(2,3-dihydrobenzofuran-5-yl)-phenyl, and 4-(benzo[1,3]dioxol-5-yl)-phenyl, 3-(2,3-dihydrobenzofuran-5-yl)-phenyl, and 3-(benzo[1,3]dioxol-5-yl)-phenyl,

R7 is hydroxyl,

and the salts of these compounds.

Compounds according to aspect A of the present invention to be more emphasized are those compounds of formula I in which

R1, R2, R3, R4 and R5 are hydrogen,

R6 is -T1-Q1, in which T1 is a bond;

Q1 is any one selected from the group consisting of

3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 3′-(morpholin-4-yl-methyl)-biphenyl-4-yl, 4′-(morpholin-4-yl-methyl)-biphenyl-4-yl, 4′-(3-morpholin-4-yl-propyl)-biphenyl-3-yl, 4′-(3-morpholin-4-yl-propyl)-biphenyl-4-yl, 3′-(3-morpholin-4-yl-propyl)-biphenyl-3-yl, 3′-(3-morpholin-4-yl-propyl)-biphenyl-4-yl,

4′-(4-methyl-piperazin-1-ylmethyl)-biphenyl-3-yl, 4′-(4-methyl-piperazin-1-ylmethyl)-biphenyl-4-yl, 3′-(4-methyl-piperazin-1-ylmethyl)-biphenyl-3-yl, 3′-(4-methyl-piperazin-1-ylmethyl)-biphenyl-4-yl, 4′-(2-morpholin-4-yl-ethoxy)-biphenyl-3-yl, 4′-(2-morpholin-4-yl-ethoxy)-biphenyl-4-yl, 3′-(2-morpholin-4-yl-ethoxy)-biphenyl-3-yl, 3′-(2-morpholin-4-yl-ethoxy)-biphenyl-4-yl, 4′-(3-morpholin-4-yl-propoxy)-biphenyl-3-yl, 4′-(3-morpholin-4-yl-propoxy)-biphenyl-4-yl, 3′-(3-morpholin-4-yl-propoxy)-biphenyl-3-yl, 3′-(3-morpholin-4-yl-propoxy)-biphenyl-4-yl, 4′-[2-(4-methyl-piperazin-1-yl)-ethoxy]-biphenyl-3-yl, 4′-[2-(4-methyl-piperazin-1-yl)-ethoxy]-biphenyl-4-yl, 3′-[2-(4-methyl-piperazin-1-yl)-ethoxy]-biphenyl-3-yl, 3′-[2-(4-methyl-piperazin-1-yl)-ethoxy]-biphenyl-4-yl, 4′-(2-pyrrolidin-1-yl-ethoxy]-biphenyl-3-yl, 4′-(2-pyrrolidin-1-yl-ethoxy]-biphenyl-4-yl, 3′-(2-pyrrolidin-1-yl-ethoxy]-biphenyl-3-yl, 3′-(2-pyrrolidin-1-yl-ethoxy]-biphenyl-4-yl, 3′-(3-pyrrolidin-1-yl-propoxy]-biphenyl-4-yl, 4′-(3-pyrrolidin-1-yl-propoxy]-biphenyl-4-yl, 3′-(3-pyrrolidin-1-yl-propoxy]-biphenyl-3-yl, 4′-(3-pyrrolidin-1-yl-propoxy]-biphenyl-3-yl,

4′-[3-(4-methyl-piperazin-1-yl)-propoxy]-biphenyl-4-yl, 3′-[3-(4-methyl-piperazin-1-yl)-propoxy]-biphenyl-4-yl, 4′-[3-(4-methyl-piperazin-1-yl)-propoxy]-biphenyl-3-yl, 3′-[3-(4-methyl-piperazin-1-yl)-propoxy]-biphenyl-3-yl, 4′-(2-(1-methyl-piperidin-4-yl)-ethoxy)-biphenyl-4-yl, 4′-(2-(1-methyl-piperidin-4-yl)-ethoxy)-biphenyl-3-yl, 3′-(2-(1-methyl-piperidin-4-yl)-ethoxy)-biphenyl-4-yl, 3′-(2-(1-methyl-piperidin-4-yl)-ethoxy)-biphenyl-3-yl, 2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 3′-dimethylaminomethyl-biphenyl-4-yl, 3′-dimethylaminomethyl-biphenyl-3-yl, 3′-[(2-dimethylamino-ethylamino)-carbonyl]-biphenyl-4-yl, 4′-[(2-dimethylamino-ethylamino)-carbonyl]-biphenyl-4-yl, 4′-[(2-dimethylamino-ethylamino)-carbonyl]-biphenyl-3-yl, 3′-[(2-dimethylamino-ethylamino)-carbonyl]-biphenyl-3-yl, 2′-methylsulphonylamino-biphenyl-4-yl, 3′-methylsulphonylamino-biphenyl-4-yl, 4′-methylsulphonylamino-biphenyl-4-yl, 2′-methylsulphonylamino-biphenyl-3-yl, 3′-methylsulphonylamino-biphenyl-3-yl, 4′-methylsulphonylamino-biphenyl-3-yl, 4′-methylsulphonylamino-biphenyl-3-yl,

4′-dimethylsulphamoyl-biphenyl-4-yl, 4′-dimethylsulphamoyl-biphenyl-3-yl, 3′-dimethylsulphamoyl-biphenyl-4-yl, 3′-dimethylsulphamoyl-biphenyl-3-yl, 3′-acetamido-biphenyl-4-yl, 4′-acetamido-biphenyl-4-yl, 3′-acetamido-biphenyl-3-yl, 4′-acetamido-biphenyl-3-yl, 3′-amino-biphenyl-4-yl, 3′-dimethylamino-biphenyl-4-yl, 4′-morpholin-4-yl-biphenyl-4-yl, 4′-hydroxy-biphenyl-4-yl, 3′-trifluoromethyl-biphenyl-4-yl, 4′-methoxy-biphenyl-4-yl, 3′-amino-biphenyl-3-yl, 3′-dimethylamino-biphenyl-3-yl, 4′-morpholin-4-yl-biphenyl-3-yl, 4′-hydroxy-biphenyl-3-yl, 3′-trifluoromethyl-biphenyl-3-yl, 4′-methoxy-biphenyl-3-yl, 4′-amino-biphenyl-4-yl, 4′-dimethylamino-biphenyl-4-yl, 3′-morpholin-4-yl-biphenyl-4-yl, 3′-hydroxy-biphenyl-4-yl, 4′-trifluoromethyl-biphenyl-4-yl, 3′-methoxy-biphenyl-4-yl, 4′-amino-biphenyl-3-yl, 4′-dimethylamino-biphenyl-3-yl, 3′-morpholin-4-yl-biphenyl-3-yl, 3′-hydroxy-biphenyl-3-yl, 4′-trifluoromethyl-biphenyl-3-yl and 3′-methoxy-biphenyl-3-yl, 4′-(2-methoxy-ethylamino)methyl-biphenyl-3-yl, 4′-(2-methoxy-ethylamino)methyl-biphenyl-4-yl, 3′-(2-methoxy-ethylamino)methyl-biphenyl-3-yl, 3′-(2-methoxy-ethylamino)methyl-biphenyl-4-yl, 4′-aminomethyl-biphenyl-3-yl, 4′-aminomethyl-biphenyl-4-yl, 3′-aminomethyl-biphenyl-3-yl, 3′-aminomethyl-biphenyl-4-yl, 4′-(acetylamino)-methyl-biphenyl-4-yl, 4′-(methylsulphonylamino)-methyl-biphenyl-4-yl, 3′-(acetylamino)-methyl-biphenyl-3-yl, 3′-(methylsulphonylamino)-methyl-biphenyl-3-yl, 4′-(acetylamino)-methyl-biphenyl-3-yl, 4′-(methylsulphonylamino)-methyl-biphenyl-3-yl, 3′-(acetylamino)-methyl-biphenyl-4-yl, 3′-(methylsulphonylamino)-methyl-biphenyl-4-yl, 4′-cyclopentylaminomethyl-biphenyl-4-yl, 4′-cyclopentylaminomethyl-biphenyl-3-yl, 3′-cyclopentylaminomethyl-biphenyl-4-yl, 3′-cyclopentylaminomethyl-biphenyl-3-yl, 4′-cyclopropylaminomethyl-biphenyl-3-yl, 4′-cyclopropylaminomethyl-biphenyl-4-yl, 3′-cyclopropylaminomethyl-biphenyl-3-yl, 3′-cyclopropylaminomethyl-biphenyl-4-yl, 3′-hydroxymethyl-biphenyl-4-yl, 3′-hydroxymethyl-biphenyl-3-yl,

4′-hydroxymethyl-biphenyl-4-yl, 4′-hydroxymethyl-biphenyl-3-yl, 5-[2-(4-methyl-piperazin-1-yl)-pyridin-4-yl]-thiophen-2-yl, 5-(1-methyl-pyrazol-4-yl)-thiophen-2-yl, 6-(1-methyl-pyrazol-4-yl)-pyridin-3-yl,

2′-(4-methyl-piperazin-1-yl)-2,4′-bipyridyl-5-yl, 5-(2-methyl-thiazol-4-yl)-thiophen-2-yl,

5-[4-(2-morpholin-4-yl-ethyl)-phenyl]-thiophen-2-yl, 5-[3-(2-morpholin-4-yl-ethyl)-phenyl]-thiophen-2-yl,

5-[4-(morpholin-4-yl-methyl)-phenyl]-thiophen-2-yl, 5-[3-(morpholin-4-yl-methyl)-phenyl]-thiophen-2-yl,

5-[4-(2-morpholin-4-yl-ethoxy)-phenyl]-thiophen-2-yl, 5-[3-(2-morpholin-4-yl-ethoxy)-phenyl]-thiophen-2-yl, 5-[4-(3-morpholin-4-yl-propoxy)-phenyl]-thiophen-2-yl, 5-[3-(3-morpholin-4-yl-propoxy)-phenyl]-thiophen-2-yl, 5-{4-[2-(4-methyl-piperazin-1-yl)-ethoxy]-phenyl}-thiophen-2-yl, 5-{3-[2-(4-methyl-piperazin-1-yl)-ethoxy]-phenyl}-thiophen-2-yl, 5-[4-(2-pyrrolidin-1-yl-ethoxy)-phenyl]-thiophen-2-yl, 5-[3-(2-pyrrolidin-1-yl-ethoxy)-phenyl]-thiophen-2-yl, 5-(4-dimethylaminomethyl-phenyl)-thiophen-2-yl, 5-(3-dimethylaminomethyl-phenyl)-thiophen-2-yl, 6-(4-dimethylaminomethyl-phenyl)-pyridin-3-yl, 6-(3-dimethylaminomethyl-phenyl)-pyridin-3-yl, 6-[4-(2-pyrrolidin-1-yl-ethoxy)-phenyl]-pyridin-3-yl, 6-[3-(2-pyrrolidin-1-yl-ethoxy)-phenyl]-pyridin-3-yl, 5-(3-aminomethyl-phenyl)-thiophen-2-yl, 5-[3-(acetylamino)-methyl-phenyl]-thiophen-2-yl, 5-[3-(methylsulphonylamino)-methyl-phenyl]-thiophen-2-yl, 5-(4-dimethylsulphamoyl-phenyl)-thiophen-2-yl, 5-(4-aminomethyl-phenyl)-thiophen-2-yl, 5-[4-(acetylamino)-methyl-phenyl]-thiophen-2-yl, 5-[4-(methylsulphonylamino)-methyl-phenyl]-thiophen-2-yl, 5-(3-dimethylsulphamoyl-phenyl)-thiophen-2-yl, 4-[2-(4-methyl-piperazin-1-yl)-pyridin-4-yl]-phenyl, 3-[2-(4-methyl-piperazin-1-yl)-pyridin-4-yl]-phenyl, 4-(6-amino-pyridin-3-yl)-phenyl, 3-(6-amino-pyridin-3-yl)-phenyl, 4-(6-methoxy-pyridin-3-yl)-phenyl, 3-(6-methoxy-pyridin-3-yl)-phenyl,

3-(1-methyl-pyrazol-4-yl)-phenyl, 4-(1-methyl-pyrazol-4-yl)-phenyl, 4-(3,5-dimethyl-isoxazol-4-yl)-phenyl, 3-(3,5-dimethyl-isoxazol-4-yl)-phenyl, 4-(1-methyl-indol-5-yl)-phenyl, 3-(1-methyl-indol-5-yl)-phenyl,

4-{1-(2-morpholin-4-yl-ethyl)-[1,2,3]triazol-4-yl}-phenyl, 4-{1-(2-piperidin-1-yl-ethyl)-[1,2,3]triazol-4-yl}-phenyl, 3-{1-(2-morpholin-4-yl-ethyl)-[1,2,3]triazol-4-yl}-phenyl, 3-{1-(2-piperidin-1-yl-ethyl)-[1,2,3]triazol-4-yl}-phenyl, 4-(2,3-dihydrobenzofuran-5-yl)-phenyl, and 4-(benzo[1,3]dioxol-5-yl)-phenyl, 3-(2,3-dihydrobenzofuran-5-yl)-phenyl, and 3-(benzo[1,3]dioxol-5-yl)-phenyl,

R7 is 2-aminophenyl,

and the salts of these compounds.

Compounds according to aspect B of the present invention to be more emphasized are those compounds of formula I in which
R1, R2, R3, R4 and R5 are independently hydrogen or 1-4C-alkyl,
R6 is -T1-Q1, in which T1 is a bond;
either
Q1 is substituted by R61 and/or R62, and is Aa1, Hh1, Ha1, Ha2, Ha3 or Ah1,
Or Q1 is unsubstituted, and is Ha2 or Ha3,
in which
R61 is 1-4C-alkyl, 1-4C-alkoxy, halogen, hydroxy-1-4C-alkyl, 1-4C-alkylsulphonylamino, tolylsulphonylamino, phenylsulphonylamino, 1-4C-alkylcarbonylamino, di-1-4C-alkylaminosulphonyl, -T2-N(R611)R612, or —U-T3-N(R613)R614, in which
T2 is a bond or 1-4C-alkylene,
R611 is hydrogen, 1-4C-alkyl, 3-7C-cycloalkyl or 1-4C-alkoxy-2-4C-alkyl,
R612 is hydrogen or 1-4C-alkyl,
or R611 and R612 together and with inclusion of the nitrogen atom, to which they are bonded, form a heterocyclic ring Het1, in which Het1 is morpholino, piperidino, pyrrolidino, piperazino or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl or 1-4C-alkoxy-2-4C-alkyl,
R614 is hydrogen or 1-4C-alkyl,
or R613 and R614 together and with inclusion of the nitrogen atom, to which they are bonded, form a heterocyclic ring Het2, in which
Het2 is morpholino, piperidino, pyrrolidino, piperazino or 4N-(1-4C-alkyl)-piperazino,
R62 is 1-4C-alkyl,
Aa1 is biphenyl,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an phenyl-heteroaryl radical made up of an phenyl group and a heteroaryl group selected from a group consisting of monocyclic 5- or 6-membered heteroaryl radicals comprising one or two heteroatoms, each of which is selected from the group consisting of nitrogen, oxygen and sulfur, whereby said phenyl and heteroaryl groups are linked together via a single bond, and whereby Ah1 is bonded via said heteroaryl moiety to the parent molecular group,
Ha1 is a heteroaryl-phenyl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a heteroaryl-phenyl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
Ha3 is a heteroaryl-phenyl radical made up of a heteroaryl group selected from a group consisting of monocyclic 5-membered heteroaryl radicals comprising three or four heteroatoms, each of which is selected from the group consisting of nitrogen, oxygen and sulfur, and a phenyl group, whereby said heteroaryl and phenyl groups are linked together via a single bond, and whereby Ha3 is bonded via said phenyl moiety to the to the parent molecular group,
R7 is hydroxyl, or 2-aminophenyl,
and the salts of these compounds.

Compounds of formula (I) to be more emphasized are those in which
R1, R2, R3, R4 and R5 are hydrogen,
R6 is -T1-Q1, in which T1 is a bond;
Q1 is substituted by R61 and/or R62 on the terminal ring, and is Aa1, Hh1, Ha1, Ha2 or Ah1,
in which
R61 is 1-2C-alkyl, 1-2C-alkoxy, halogen, hydroxy-1-2C-alkyl, 1-2C-alkylsulphonylamino, 1-2C-alkylcarbonylamino, di-1-2C-alkylaminosulphonyl, -T2-N(R611)R612, or —U-T3-N(R613)R614, in which
T2 is a bond or straight chain 1-4C-alkylene,
R611 is hydrogen, 1-2C-alkyl, 3-5C-cycloalkyl or 1-2C-alkoxy-2-3C-alkyl,
R612 is hydrogen or 1-2C-alkyl,
or R611 and R612 together and with inclusion of the nitrogen atom, to which they are bonded, form a heterocyclic ring Het1, in which
Het1 is morpholino, piperidino, pyrrolidino, piperazino or 4N-(1-2C-alkyl)-piperazino,
U is —O— (oxygen) or —C(O)NH—,
T3 is straight chain 2-4C-alkylene,
R613 is hydrogen, 1-2C-alkyl, 3-5C-cycloalkyl or 1-2C-alkoxy-2-3C-alkyl,
R614 is hydrogen or 1-2C-alkyl,
or R613 and R614 together and with inclusion of the nitrogen atom, to which they are bonded, form a heterocyclic ring Het2, in which
Het2 is morpholino, piperidino, pyrrolidino, piperazino or 4N-(1-4C-alkyl)-piperazino,
R62 is 1-2C-alkyl,
Aa1 is 1,1′-biphenyl-3-yl or 1,1′-biphenyl-4-yl,
Hh1 is a bisheteroaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a thiophenyl group, whereby said heteroaryl and thiophenyl groups are linked together via a single bond, and whereby Hh1 is bonded via said thiophenyl moiety to the to the parent molecular group,
Ah1 is phenyl-thiophenyl,
Ha1 is a 3-(heteroaryl)-phenyl or 4-(heteroaryl)-phenyl radical each made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a 3-(heteroaryl)-phenyl or 4-(heteroaryl)-phenyl radical each made up of a heteroaryl group selected from a group consisting of fused bicyclic 9- or 10-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
R7 is hydroxyl, or 2-aminophenyl,
and the salts of these compounds.

Compounds of formula I to be more emphasized are those in which
R1, R2, R3, R4 and R5 are hydrogen,
R6 is -T1-Q1, in which T1 is a bond;
Q1 is substituted by R61 and/or R62 on the terminal ring, and is Aa1, Hh1, Ha1, Ha2 or Ah1,
in which
R61 is 1-2C-alkyl, 1-2C-alkoxy, hydroxyl, trifluoromethyl, halogen, hydroxy-1-2C-alkyl, 1-2C-alkylsulphonylamino, 1-2C-alkylcarbonylamino, di-1-2C-alkylaminosulphonyl, -T2-N(R611)R612, or —U-T3-N(R613)R614, in which
T2 is a bond or straight chain 1-4C-alkylene,
R611 is hydrogen, 1-2C-alkyl, 3-5C-cycloalkyl or 1-2C-alkoxy-2-3C-alkyl,
R612 is hydrogen or 1-2C-alkyl,
or R611 and R612 together and with inclusion of the nitrogen atom, to which they are bonded, form a heterocyclic ring Het1, in which
Het1 is morpholino, piperidino, pyrrolidino, piperazino or 4N-(1-2C-alkyl)-piperazino,
U is —O— (oxygen) or —C(O)NH—,
T3 is straight chain 2-4C-alkylene,
R613 is hydrogen, 1-2C-alkyl, 3-5C-cycloalkyl or 1-2C-alkoxy-2-3C-alkyl,
R614 is hydrogen or 1-2C-alkyl,
or R613 and R614 together and with inclusion of the nitrogen atom, to which they are bonded, form a heterocyclic ring Het2, in which
Het2 is morpholino, piperidino, pyrrolidino, piperazino or 4N-(1-4C-alkyl)-piperazino,
R62 is 1-2C-alkyl,
Aa1 is 1,1′-biphenyl-3-yl or 1,1′-biphenyl-4-yl,
Hh1 is a bisheteroaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a thiophenyl group, whereby said heteroaryl and thiophenyl groups are linked together via a single bond, and whereby Hh1 is bonded via said thiophenyl moiety to the to the parent molecular group,
Ah1 is phenyl-thiophenyl or phenyl-pyridinyl,
Ha1 is a 3-(heteroaryl)-phenyl or 4-(heteroaryl)-phenyl radical each made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a 3-(heteroaryl)-phenyl or 4-(heteroaryl)-phenyl radical each made up of a heteroaryl group selected from a group consisting of fused bicyclic 9- or 10-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
R7 is hydroxyl, or 2-aminophenyl,
and the salts of these compounds.
Compounds of formula I to be more emphasized are those compounds in which
R1, R2, R3, R4 and R5 are hydrogen,
R6 is -T1-Q1, in which T1 is a bond;
Q1 is substituted by R61 on the terminal ring, and is Aa1 or Ah1, in which
Aa1 is 1,1′-biphenyl-3-yl or 1,1′-biphenyl-4-yl,
Ah1 is phenyl-thiophenyl,
R61 is methoxy, hydroxymethyl, methylsulphonylamino, methylcarbonylamino, dimethylaminosulphonyl, -T2-N(R611)R612, or —U-T3-N(R613)R614, in which
T2 is a bond, methylene, dimethylene or trimethylene,
R611 is hydrogen, methyl, cyclopropyl or 2-methoxyethyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino, piperazino or 4N-methyl-piperazino,
U is —O— (oxygen) or —C(O)NH—,
T3 is dimethylene or trimethylene,
R613 is hydrogen, methyl, cyclopropyl or 2-methoxyethyl,
R614 is hydrogen or methyl,
or R613 and R614 together and with inclusion of the nitrogen atom, to which they are bonded, form a heterocyclic ring Het2, in which
Het2 is morpholino, piperidino, pyrrolidino, piperazino or 4N-methyl-piperazino,
or
Q1 is substituted by R61 on the terminal ring, and is Hh1 or Ha1, in which
Hh1 is pyridinyl-thiophenyl,
Ha1 is 3-(pyridinyl)-phenyl or 4-(pyridinyl)-phenyl,
R61 is methoxy, or -T2-N(R611)R612, in which
T2 is a bond, methylene, dimethylene or trimethylene,
R611 is hydrogen, methyl, cyclopropyl or 2-methoxyethyl,
R612 is hydrogen or methyl,
or R611 and R612 together and with inclusion of the nitrogen atom, to which they are bonded, form a heterocyclic ring Het1, in which Het1 is morpholino, piperidino, pyrrolidino, piperazino or 4N-methyl-piperazino,
or
Q1 is 3-(1N-methyl-pyrazolyl)-phenyl, 4-(1N-methyl-pyrazolyl)-phenyl, 3-(1N-methyl-indolyl)-phenyl or 4-(1N-methyl-indolyl)-phenyl,
R7 is hydroxyl, or 2-aminophenyl,
and the salts of these compounds.

Yet compounds of formula I may be those compounds in which
R1, R2, R3, R4 and R5 are all hydrogen,
R6 is -T1-Q1, in which T1 is a bond,
Q is (1N-methyl-pyrazolyl)-thiophenyl, 3-(dimethyl-isoxazolyl)-phenyl or 4-(dimethyl-isoxazolyl)-phenyl,
and the salts of these compounds.

In another embodiment, still yet compounds of formula I in more particular worthy to be mentioned are those in which
R1, R2, R3, R4 and R5 are all hydrogen,
R6 is -T1-Q1, in which T1 is a bond,
Q1 is substituted by R61 on the terminal ring, and is Aa1 or Ah1, in which
Aa1 is 1,1′-biphenyl-3-yl or 1,1′-biphenyl-4-yl,
Ah1 is phenyl-thiophenyl or phenyl-pyridinyl,
R61 is methoxy, hydroxyl, trifluoromethyl, hydroxymethyl, methylsulphonylamino, methylcarbonylamino, dimethylaminosulphonyl, -T2-N(R611)R612, or —U-T3-N(R613)R614, in which
T2 is a bond, methylene, dimethylene or trimethylene,
R611 is hydrogen, methyl, cyclopropyl, cyclopentyl or 2-methoxyethyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino, piperazino or 4N-methyl-piperazino,
U is —O— (oxygen) or —C(O)NH—,
T3 is dimethylene or trimethylene,
R613 is hydrogen, methyl, cyclopropyl, cyclopentyl or 2-methoxyethyl,
R614 is hydrogen or methyl,
or R613 and R614 together and with inclusion of the nitrogen atom, to which they are bonded, form a heterocyclic ring Het2, in which
Het2 is morpholino, piperidino, pyrrolidino, piperazino or 4N-methyl-piperazino,
or
Q1 is substituted by R61 on the terminal ring, and is Hh1 or Ha1, in which
Hh1 is pyridinyl-thiophenyl or bipyridyl,
Ha1 is 3-(pyridinyl)-phenyl or 4-(pyridinyl)-phenyl,
R61 is methoxy, or -T2-N(R611)R612, in which
T2 is a bond, methylene, dimethylene or trimethylene,
R611 is hydrogen, methyl, cyclopropyl, cyclopentyl or 2-methoxy ethyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino, piperazino or 4N-methyl-piperazino,
or
Q1 is 3-(1N-methyl-pyrazolyl)-phenyl, 4-(1N-methyl-pyrazolyl)-phenyl, (1N-methyl-pyrazolyl)-thiophenyl, (1N-methyl-pyrazolyl)-pyridinyl, 3-(methyl-thiazolyl)-phenyl, 4-(methyl-thiazolyl)-phenyl, (methyl-thiazolyl)-thiophenyl, (methyl-thiazolyl)-pyridinyl, 3-(dimethyl-isoxazolyl)-phenyl, 4-(dimethyl-isoxazolyl)-phenyl, 3-(1N-methyl-indolyl)-phenyl or 4-(1N-methyl-indolyl)-phenyl,
R7 is hydroxyl, or 2-aminophenyl,
and the salts of these compounds.

Compounds of formula I to be emphasized are those in which
R1, R2, R3, R4 and R5 are all hydrogen,
R6 is -T1-Q1, in which T1 is a bond,
Q1 is substituted by R61 on the terminal ring, and is Aa1 or Ah1, in which
Aa1 is 1,1′-biphenyl-3-yl or 1,1′-biphenyl-4-yl,
Ah1 is phenyl-thiophenyl,
R61 is hydroxymethyl, methylsulphonylamino, methylcarbonylamino, dimethylaminosulphonyl, -T2-N(R611)R612, or —U-T3-N(R613)R614, in which
T2 is methylene, dimethylene or trimethylene,
R611 is methyl, cyclopropyl or 2-methoxy ethyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino,
U is —O— (oxygen) or —C(O)NH—,
T3 is dimethylene or trimethylene,
R613 and R614 are methyl,
or R613 and R614 together and with inclusion of the nitrogen atom, to which they are bonded, form a heterocyclic ring Het2, in which Het2 is morpholino, piperidino, pyrrolidino or 4N-methyl-piperazino,
or
Q1 is substituted by R61 on the terminal ring, and is Hh1 or Ha1, in which
Hh1 is pyridinyl-thiophenyl,
Ha1 is 3-(pyridinyl)-phenyl or 4-(pyridinyl)-phenyl,
R61 is methoxy, or -T2-N(R611)R612, in which
T2 is a bond,
R611 and R612 are independently is hydrogen or methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino,
or
Q1 is 3-(1N-methyl-pyrazolyl)-phenyl, 4-(1N-methyl-pyrazolyl)-phenyl, 3-(1N-methyl-indolyl)-phenyl or 4-(1N-methyl-indolyl)-phenyl,
R7 is hydroxyl, or 2-aminophenyl,
and the salts of these compounds.

Yet compounds of formula I to be emphasized are those in which
R1, R2, R3, R4 and R5 are all hydrogen,
R6 is -T1-Q1, in which T1 is a bond,
Q is (1N-methyl-pyrazol-4-yl)-thiophenyl,
   3-(dimethyl-isoxazolyl)-phenyl or 4-(dimethyl-isoxazolyl)-phenyl,
and the salts of these compounds.

In another embodiment, yet compounds of formula I to be emphasized are those in which
R1, R2, R3, R4 and R5 are all hydrogen,
R6 is -T1-Q1, in which T1 is a bond,
Q1 is 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is methoxy, hydroxyl, trifluoromethyl, hydroxymethyl, methylsulphonylamino, methylcarbonylamino, dimethylaminosulphonyl, -T2-N(R611)R612, or —U-T3-N(R613)R614, in which
T2 is a bond, methylene, dimethylene or trimethylene,
R611 is hydrogen, methyl, cyclopropyl, cyclopentyl or 2-methoxyethyl,
R612 is hydrogen or methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino,
either
U is —O— (oxygen),
T3 is dimethylene or trimethylene,
R613 and R614 together and with inclusion of the nitrogen atom, to which they are bonded, form a heterocyclic ring Het2, in which
Het2 is morpholino, piperidino, pyrrolidino or 4N-methyl-piperazino,
or
U is —C(O)NH—,
T3 is dimethylene or trimethylene,
R613 and R614 are methyl,
or
Q1 is 5-[3-(R61)-phenyl]-thiophen-2-yl, 5-[4-(R61)-phenyl]-thiophen-2-yl, 2-[3-(R61)-phenyl]-pyridin-4-yl, 2-[4-(R61)-phenyl]-pyridin-4-yl, 6-[3-(R61)-phenyl]-pyridin-3-yl or 6-[4-(R61)-phenyl]-pyridin-3-yl, in which
R61 is methoxy, hydroxyl, trifluoromethyl, hydroxymethyl, methylsulphonylamino, methylcarbonylamino, dimethylaminosulphonyl, -T2-N(R611)R612, or —U-T3-N(R613)R614, in which T2 is a bond, methylene, dimethylene or trimethylene,
either
R611 is methyl, cyclopropyl, cyclopentyl or 2-methoxy ethyl,
R612 is hydrogen,
or R611 and R612 are hydrogen,
or R611 and R612 are methyl,
or R611 and R612 together and with inclusion of the nitrogen atom, to which they are bonded, form a heterocyclic ring Het1, in which
Het1 is morpholino, piperidino, pyrrolidino or 4N-methyl-piperazino,
either
U is —O— (oxygen),
T3 is dimethylene or trimethylene,
R613 and R614 together and with inclusion of the nitrogen atom, to which they are bonded, form a heterocyclic ring Het2, in which
Het2 is morpholino, piperidino, pyrrolidino or 4N-methyl-piperazino,
or
Q1 is 5-[2-(R61)-pyridin-4-yl]-thiophen-2-yl or 5-[6-(R61)-pyridin-3-yl]-thiophen-2-yl, in which
R61 is amino, methoxy, dimethylamino, or -T2-N(R611)R612, in which
T2 is a bond,
R611 and R612 together and with inclusion of the nitrogen atom, to which they are bonded, form a heterocyclic ring Het1, in which
Het1 is morpholino, piperidino, pyrrolidino or 4N-methyl-piperazino,
or
Q1 is 2-[2-(R61)-pyridin-4-yl]-pyridin-4-yl, 2-[6-(R61)-pyridin-3-yl]-pyridin-4-yl, 3-[2-(R61)- pyridin-4-yl]-pyridin-6-yl or 3-[6-(R61)-pyridin-3-yl]-pyridin-6-yl, in which
R61 is amino, methoxy, dimethylamino, or -T2-N(R611)R612, in which
T2 is a bond,
R611 and R612 together and with inclusion of the nitrogen atom, to which they are bonded, form a heterocyclic ring Het1, in which
Het1 is morpholino, piperidino, pyrrolidino or 4N-methyl-piperazino,
or
Q1 is 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl, in which
R61 is amino, methoxy, dimethylamino, or -T2-N(R611)R612, in which
T2 is a bond,
R611 and R612 together and with inclusion of the nitrogen atom, to which they are bonded, form a heterocyclic ring Het1, in which
Het1 is morpholino, piperidino, pyrrolidino or 4N-methyl-piperazino,
or
Q1 is 3-(1N-methyl-pyrazol-4-yl)-phenyl, 4-(1N-methyl-pyrazol-4-yl)-phenyl, 5-(1N-methyl-pyrazol-4-yl)-thiophen-2-yl, 6-(1N-methyl-pyrazol-4-yl)-pyridin-3-yl, 5-(2-methyl-thiazol-4-yl)-thiophen-2-yl, 3-(3,5-dimethyl-isoxazol-4-yl)-phenyl, 4-(3,5-dimethyl-isoxazol-4-yl)-phenyl, 3-(1N-methyl-indol-5-yl)-phenyl or 4-(1N-methyl-indol-5-yl)-phenyl,
R7 is hydroxyl, or 2-aminophenyl,
and the salts of these compounds.

Compounds of formula I to be emphasized are those in which
in which
R1, R2, R3, R4 and R5 are all hydrogen,
R6 is -T1-Q1, in which T1 is a bond,
Q1 is 2′-(R61)-1,1′-biphenyl-3-yl, 2′-(R61)-1,1′-biphenyl-4-yl, 3′-(R61)-1,1′-biphenyl-3-yl, 3′-(R61)-1,1′-biphenyl-4-yl, 4′-(R61)-1,1′-biphenyl-3-yl or 4′-(R61)-1,1′-biphenyl-4-yl, in which
R61 is hydroxymethyl, methylsulphonylamino, methylcarbonylamino, dimethylaminosulphonyl, -T2-N(R611)R612, or —U-T3-N(R613)R614, in which
T2 is methylene, dimethylene or trimethylene,
R611 is methyl, cyclopropyl or 2-methoxyethyl,
R612 is hydrogen or methyl,
or R611 and R612 together and with inclusion of the nitrogen atom, to which they are bonded, form a heterocyclic ring Het1, in which Het1 is morpholino, pyrrolidino or 4N-methyl-piperazino,
either
U is —O— (oxygen),
T3 is dimethylene or trimethylene,
R613 and R614 together and with inclusion of the nitrogen atom, to which they are bonded, form a heterocyclic ring Het2, in which
Het2 is morpholino, pyrrolidino or 4N-methyl-piperazino,
or
U is —C(O)NH—,
T3 is dimethylene or trimethylene,
R613 and R614 aremethyl,
or
Q1 is 5-[3-(R61)-phenyl]-thiophen-2-yl or 5-[4-(R61)-phenyl]-thiophen-2-yl, in which
R61 is -T2-N(R611)R612, or —U-T3-N(R613)R614, in which
T2 is methylene, dimethylene or trimethylene,
R611 and R612 are methyl,
or R611 and R612 together and with inclusion of the nitrogen atom, to which they are bonded, form a heterocyclic ring Het1, in which
Het1 is morpholino, pyrrolidino or 4N-methyl-piperazino,
U is —O— (oxygen),
T3 is dimethylene or trimethylene,
R613 and R614 together and with inclusion of the nitrogen atom, to which they are bonded, form a heterocyclic ring Het2, in which Het2 is morpholino, pyrrolidino or 4N-methyl-piperazino,
or
Q1 is 5-[2-(R61)-pyridin-4-yl]-thiophen-2-yl or 5-[6-(R61)-pyridin-3-yl]-thiophen-2-yl, in which
R61 is amino, or -T2-N(R611)R612, in which T2 is a bond,
R611 and R612 together and with inclusion of the nitrogen atom, to which they are bonded, form a heterocyclic ring Het1, in which Het1 is morpholino, pyrrolidino or 4N-methyl-piperazino,
Q1 is 3-[2-(R61)-pyridin-4-yl]-phenyl, 4-[2-(R61)-pyridin-4-yl]-phenyl, 3-[6-(R61)-pyridin-3-yl]-phenyl or 4-[6-(R61)-pyridin-3-yl]-phenyl, in which
R61 is amino, methoxy, or -T2-N(R611)R612, in which T2 is a bond,
R611 and R612 together and with inclusion of the nitrogen atom, to which they are bonded, form a heterocyclic ring Het1, in which Het1 is morpholino, pyrrolidino or 4N-methyl-piperazino,
or
Q1 is 3-(1N-methyl-pyrazol-4-yl)-phenyl, 4-(1N-methyl-pyrazol-4-yl)-phenyl, 3-(1N-methyl-indol-5-yl)-phenyl or 4-(1N-methyl-indol-5-yl)-phenyl,
R7 is hydroxyl, or 2-aminophenyl,
and the salts of these compounds.

Compounds of formula I to be emphasized are those in which
R1, R2, R3, R4 and R5 are all hydrogen,
R6 is -T1-Q1, in which T1 is a bond,
Q is 5-(1N-methyl-pyrazol-4-yl)-thiophen-2-yl, 3-(3,5-dimethyl-isoxazol-4-yl)-phenyl or 4-(3,5-dimethyl-isoxazol-4-yl)-phenyl,
and the salts of these compounds.

Compounds of formula I to be emphasized are those in which

R1, R2, R3, R4 and R5 are all hydrogen,

R6 is -T1-Q1, in which T1 is a bond,

Q1 is any one selected from the group consisting of

3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 4′-(3-morpholin-4-yl-propyl)-biphenyl-3-yl, 4′-(4-methyl-piperazin-1-ylmethyl)-biphenyl-3-yl, 4′-(2-morpholin-4-yl-ethoxy)-biphenyl-3-yl, 4′-(3-morpholin-4-yl-propoxy)-biphenyl-3-yl, 4′-[2-(4-methyl-piperazin-1-yl)-ethoxy]-biphenyl-3-yl, 4′-(2-pyrrolidin-1-yl-ethoxy]-biphenyl-3-yl, 2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 3′-[(2-dimethylamino-ethylamino)-carbonyl]-biphenyl-4-yl, 4′-[(2-dimethylamino-ethylamino)-carbonyl]-biphenyl-4-yl, 4′-[(2-dimethylamino-ethylamino)-carbonyl]-biphenyl-3-yl, 2′-methylsulphonylamino-biphenyl-4-yl, 3′-methylsulphonylamino-biphenyl-4-yl, 4′-methylsulphonylamino-biphenyl-4-yl, 4′-dimethylsulphamoyl-biphenyl-4-yl, 3′-acetamido-biphenyl-4-yl, 4′-acetamido-biphenyl-4-yl, 4′-(2-methoxy-ethylamino)methyl-biphenyl-3-yl, 4′-cyclopropylaminomethyl-biphenyl-3-yl, 3′-hydroxymethyl-biphenyl-4-yl, 5-[2-(4-methyl-piperazin-1-yl)-pyridin-4-yl]-thiophen-2-yl, 5-(1N-methyl-pyrazol-4-yl)-thiophen-2-yl, 5-[4-(2-morpholin-4-yl-ethyl)-phenyl]-thiophen-2-yl, 5-[4-(morpholin-4-yl-methyl)-phenyl]-thiophen-2-yl, 5-[3-(morpholin-4-yl-methyl)-phenyl]-thiophen-2-yl, 5-[4-(2-morpholin-4-yl-ethoxy)-phenyl]-thiophen-2-yl,

5-[4-(3-morpholin-4-yl-propoxy)-phenyl]-thiophen-2-yl, 5-{4-[2-(4-methyl-piperazin-1-yl)-ethoxy]-phenyl}-thiophen-2-yl, 5-(4-dimethylaminomethyl-phenyl)-thiophen-2-yl, 4-[2-(4-methyl-piperazin-1-yl)-pyridin-4-yl]-phenyl, 3-[2-(4-methyl-piperazin-1-yl)-pyridin-4-yl]-phenyl, 4-[6-amino-pyridin-3-yl]-phenyl, 3-[6-amino-pyridin-3-yl]-phenyl, 4-[6-methoxy-pyridin-3-yl]-phenyl, 3-[6-methoxy-pyridin-3-yl]-phenyl,

3-(1N-methyl-pyrazol-4-yl)-phenyl, 4-(1N-methyl-pyrazol-4-yl)-phenyl, 4-(3,5-dimethyl-isoxazol-4-yl)-phenyl, and 4-(1N-methyl-indol-5-yl)-phenyl,

R7 is hydroxyl, or 2-aminophenyl,

and the salts of these compounds.

In one embodiment, compounds of formula I to be emphasized are those in which
R1, R2, R3, R4 and R5 are all hydrogen,
R6 is -T1-Q1, in which T1 is a bond,
Q1 is any one selected from the group consisting of
4′-(2-morpholin-4-yl-ethyl)-biphenyl-3-yl, 4′-(3-morpholin-4-yl-propoxy)-biphenyl-3-yl,
4′-[2-(4-methyl-piperazin-1-yl)-ethoxy]-biphenyl-3-yl, 4′-dimethylaminomethyl-biphenyl-4-yl,
5-[2-(4-methyl-piperazin-1-yl)-pyridin-4-yl]-thiophen-2-yl, 5-(4-dimethylaminomethyl-phenyl)-thiophen-2-yl,
4-[2-(4-methyl-piperazin-1-yl)-pyridin-4-yl]-phenyl, 3-[2-(4-methyl-piperazin-1-yl)-pyridin-4-yl]-phenyl,
4-[6-amino-pyridin-3-yl]-phenyl, and 4-(1N-methyl-pyrazol-4-yl)-phenyl.
R7 is hydroxyl,
and the salts of these compounds.

In another embodiment, compounds of formula I to be emphasized are those in which

R1, R2, R3, R4 and R5 are all hydrogen,

R6 is -T1-Q1, in which T1 is a bond,

Q1 is any one selected from the group consisting of

4′-(2-morpholin-4-yl-ethyl)-biphenyl-3-yl, 4′-(3-morpholin-4-yl-propoxy)-biphenyl-3-yl, 4′-[2-(4-methyl-piperazin-1-yl)-ethoxy]-biphenyl-3-yl, 4′-dimethylaminomethyl-biphenyl-4-yl, 5-[2-(4-methyl-piperazin-1-yl)-pyridin-4-yl]-thiophen-2-yl, 5-(4-dimethylaminomethyl-phenyl)-thiophen-2-yl, 4-[2-(4-methyl-piperazin-1-yl)-pyridin-4-yl]-phenyl, 3-[2-(4-methyl-piperazin-1-yl)-pyridin-4-yl]-phenyl, 4-[6-amino-pyridin-3-yl]-phenyl, and 4-(1N-methyl-pyrazol-4-yl)-phenyl,

R7 is 2-aminophenyl,

and the salts of these compounds.

In a first embodiment of aspect C (embodiment C1) of the present invention, Compounds of formula I to be emphasized are those in which
R1, R2, R3, R4, and R5 are independently hydrogen, or 1-4C-alkyl,
R6 is -T1-Q1, in which T1 is a bond,
either
Q1 is substituted by R61 and/or R62, and is Aa1, Hh1, Ha1, Ha2, Ha3 or Ah1,
or Q1 is unsubstituted, and is Ha2 or Ha3,
in which
R61 is 1-4C-alkyl, 1-4C-alkoxy, halogen, or -T2-N(R611)R612, in which
T2 is a bond or 1-4C-alkylene,
R611 and R612 are independently hydrogen or 1-4C-alkyl,
or R611 and R612 together and with inclusion of the nitrogen atom, to which they are bonded, form a heterocyclic ring Het1, in which
Het1 is morpholino, piperidino, pyrrolidino, piperazino, or 4N-methyl-piperazino,
R62 is 1-4C-alkyl,
Aa1 is biphenyl,
Hh1 is a bisheteroaryl radical made up of two heteroaryl groups,
   which are selected independently from a group consisting of monocyclic 5- or 6-membered heteroaryl radicals comprising one or two heteroatoms, each of which is selected from the group consisting of nitrogen, oxygen and sulfur, and
   which are linked together via a single bond,
Ah1 is an phenyl-heteroaryl radical made up of an phenyl group and a heteroaryl group selected from a group consisting of monocyclic 5- or 6-membered heteroaryl radicals comprising one or two heteroatoms, each of which is selected from the group consisting of nitrogen, oxygen and sulfur, whereby said phenyl and heteroaryl groups are linked together via a single bond, and whereby Ah1 is bonded via said heteroaryl moiety to the parent molecular group,
Ha1 is a heteroaryl-phenyl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a heteroaryl-phenyl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
Ha3 is a heteroaryl-phenyl radical made up of a heteroaryl group selected from a group consisting of monocyclic 5-membered heteroaryl radicals comprising three or four heteroatoms, each of which is selected from the group consisting of nitrogen, oxygen and sulfur, and a phenyl group, whereby said heteroaryl and phenyl groups are linked together via a single bond, and whereby Ha3 is bonded via said phenyl moiety to the to the parent molecular group,
R7 is hydroxyl, or 2-aminophenyl,
and the salts of these compounds.

In a second embodiment of aspect C (embodiment C2), compounds of formula I to be emphasized are those in which
R1, R2, R3, R4 and R5 are independently hydrogen, or 1-4C-alkyl,
R6 is -T1-Q1, in which T1 is a bond,
either
Q1 is substituted by R61, and is Aa1, Ha1, Ha2 or Ha3,
Or Q1 is unsubstituted, and is Ha2 or Ha3,
in which
R61 is 1-4C-alkyl, 1-4C-alkoxy, halogen, or -T2-N(R611)R612, in which
T2 is a bond or 1-4C-alkylene,
R611 and R612 are independently hydrogen or 1-4C-alkyl,
or R611 and R612 together and with inclusion of the nitrogen atom, to which they are bonded, form a heterocyclic ring Het1, in which
Het1 is morpholino, piperidino, pyrrolidino, piperazino, or 4N-methyl-piperazino,
Aa1 is biphenyl,
Ha1 is a heteroaryl-phenyl radical made up of a heteroaryl group selected from a group consisting of monocyclic 5- or 6-membered heteroaryl radicals comprising one or two heteroatoms, each of which is selected from the group consisting of nitrogen, oxygen and sulfur, and a phenyl group, whereby said heteroaryl and phenyl groups are linked together via a single bond, and whereby Ha1 is bonded via said phenyl moiety to the to the parent molecular group,
Ha2 is a heteroaryl-phenyl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and a phenyl group, whereby said heteroaryl and phenyl groups are linked together via a single bond, and whereby Ha2 is bonded via said phenyl moiety to the to the parent molecular group,
Ha3 is a heteroaryl-phenyl radical made up of a heteroaryl group selected from a group consisting of monocyclic 5-membered heteroaryl radicals comprising three or four heteroatoms, each of which is selected from the group consisting of nitrogen, oxygen and sulfur, and a phenyl group, whereby said heteroaryl and phenyl groups are linked together via a single bond, and whereby Ha3 is bonded via said phenyl moiety to the to the parent molecular group,
R7 is hydroxyl, or 2-aminophenyl,
and the salts of these compounds.

Compounds according to embodiment C1 of aspect C worthy to be mentioned are those compounds of formula I in which
R1, R2, R3, R4 and R5 are hydrogen,
R6 is -T1-Q1, in which T1 is a bond,
either
Q1 is substituted by R61 on the terminal ring, and is Aa1, Hh1, Ha1 or Ah1,
or Q1 is [1N-(1-4C-alkyl)-indolyl]-phenyl, [1N-(1-4C-alkyl)-pyrazolyl]-phenyl, [1N-(1-4C-alkyl)-imidazolyl]-phenyl, [1N-(1-4C-alkyl)-triazolyl]-phenyl, [1N-(1-4C-alkyl)-tetrazolyl]-phenyl, [1N-(1-4C-alkyl)-benzimidazolyl]-phenyl, [1N-(1-4C-alkyl)-benztriazolyl]-phenyl, or [1N-(1-4C-alkyl)-indazol]-phenyl,
or Q1 is [1N-(1-4C-alkyl)-indolyl]-thiophenyl, [1N-(1-4C-alkyl)-pyrazolyl]-thiophenyl, [1N-(1-4C-alkyl)-imidazolyl]-thiophenyl, [1N-(1-4C-alkyl)-triazolyl]-thiophenyl, [1N-(1-4C-alkyl)-tetrazolyl]-thiophenyl, [1N-(1-4C-alkyl)-benzimidazolyl]-thiophenyl, [1N-(1-4C-alkyl)-benztriazolyl]-thiophenyl, or [1N-(1-4C-alkyl)-indazol]-thiophenyl,
or Q1 is [mono- or di-(1-4C-alkyl)-isoxazolyl]-phenyl, or [mono- or di-(1-4C-alkyl)-isoxazolyl]-thiophenyl,
in which
R61 is 1-4C-alkyl, 1-4C-alkoxy, halogen, or -T2-N(R611)R612, in which
T2 is a bond or 1-4C-alkylene,
R611 is hydrogen or 1-4C-alkyl,
R612 is hydrogen or 1-4C-alkyl,
or R611 and R612 together and with inclusion of the nitrogen atom, to which they are bonded, form a heterocyclic ring Het1, in which
Het1 is morpholino, piperidino, pyrrolidino, piperazino, or 4N-methyl-piperazino,
Aa1 is 1,1′-biphenyl-4-yl or 1,1′-biphenyl-3-yl,
Hh1 is pyridinyl-thiophenyl,
Ha1 is 3-(pyridinyl)-phenyl or 4-(pyridinyl)-phenyl,
Ah1 is phenyl-thiophenyl,
R7 is hydroxyl, or 2-aminophenyl,
and the salts of these compounds.

Compounds according to embodiment C2 of aspect C worthy to be mentioned are those compounds of formula I in which
R1, R2, R3, R4 and R5 are hydrogen,
R6 is -T1-Q1, in which T1 is a bond,
either
Q1 is substituted by R61 on the terminal ring, and is Aa1 or Ha1,
or Q1 is [1N-(1-4C-alkyl)-indolyl]-phenyl, [1N-(1-4C-alkyl)-pyrazolyl]-phenyl, [1N-(1-4C-alkyl)-imidazolyl]-phenyl, [1N-(1-4C-alkyl)-triazolyl]-phenyl, [1N-(1-4C-alkyl)-tetrazolyl]-phenyl, [1N-(1-4C-alkyl)-benzimidazolyl]-phenyl, [1N-(1-4C-alkyl)-benztriazolyl]-phenyl, or [1N-(1-4C-alkyl)-indazol]-phenyl,
in which
R61 is 1-4C-alkyl, 1-4C-alkoxy, halogen, or -T2-N(R611)R612, in which
T2 is a bond or 1-4C-alkylene,
R611 is hydrogen or 1-4C-alkyl,
R612 is hydrogen or 1-4C-alkyl,
or R611 and R612 together and with inclusion of the nitrogen atom, to which they are bonded, form a heterocyclic ring Het1, in which
Het1 is morpholino, piperidino, pyrrolidino, piperazino, or 4N-methyl-piperazino,
Aa1 is 1,1′-biphenyl-4-yl or 1,1′-biphenyl-3-yl,
Ha1 is 3-(pyridinyl)-phenyl or 4-(pyridinyl)-phenyl,
R7 is hydroxyl, or 2-aminophenyl,
and the salts of these compounds.

Compounds according to embodiment C1 of aspect C worthy to be mentioned are those compounds of formula I in which R1, R2, R3, R4 and R5 are hydrogen,
R6 is -T1-Q1, in which T1 is a bond,
either
Q1 is substituted by R61 on the pyridine ring, and is 3-(pyridinyl)-phenyl or 4-(pyridinyl)-phenyl,
or Q1 is 2′-(R61)-1,1′-biphenyl-4-yl, 3′-(R61)-1,1′-biphenyl-4-yl, 4′-(R61)-1,1′-biphenyl-4-yl, T-(R61)-1,1′-biphenyl-3-yl, 3′-(R61)-1,1′-biphenyl-3-yl or 4′-(R61)-1,1′-biphenyl-3-yl,
or Q1 is substituted by R61 on the pyridine ring, and is pyridinyl-thiophenyl, or Q1 is substituted by R61 on the phenyl ring, and is phenyl-thiophenyl,
or Q1 is 3-[1N-methyl-indolyl]-phenyl, 4-[1N-methyl-indolyl]-phenyl, 3-[1N-methyl-pyrazolyl]-phenyl or 4-[1N-methyl-pyrazolyl]-phenyl,
or Q1 is [1N-methyl-pyrazolyl]-thiophenyl,
or Q1 is 3-[dimethyl-isoxazolyl]-phenyl or 4-[dimethyl-isoxazolyl]-phenyl,
in which
R61 is 1-2C-alkoxy, amino, or -T2-N(R611)R612, in which
T2 is a bond, methylene, dimethylene or trimethylene,
R611 is 1-2C-alkyl,
R612 is 1-2C-alkyl,
or R611 and R612 together and with inclusion of the nitrogen atom, to which they are bonded, form a heterocyclic ring Het1, in which
Het1 is morpholino, pyrrolidino or 4N-methyl-piperazino,
R7 is hydroxyl, or 2-aminophenyl,
and the salts of these compounds.

Compounds according to embodiment C2 of aspect C worthy to be mentioned are those compounds of formula I in which
R1, R2, R3, R4 and R5 are hydrogen,
R6 is -T1-Q1, in which T1 is a bond,
either
Q1 is substituted by R61 on the pyridine ring, and is 3-(pyridinyl)-phenyl or 4-(pyridinyl)-phenyl,
or Q1 is 3′-(R61)-1,1′-biphenyl-4-yl or 4′-(R61)-1,1′-biphenyl-4-yl,
or Q1 is 3-[1N-methyl-indolyl]-phenyl, 4-[1N-methyl-indolyl]-phenyl, 3-[1N-methyl-pyrazolyl]-phenyl or 4-[1N-methyl-pyrazolyl]-phenyl,
in which
R61 is 1-2C-alkoxy, amino, or -T2-N(R611)R612, in which
T2 is a bond or 1-2C-alkylene,
R611 and R612 are 1-2C-alkyl,
or R611 and R612 together and with inclusion of the nitrogen atom, to which they are bonded, form a heterocyclic ring Het1, in which
Het1 is morpholino,
R7 is hydroxyl, or 2-aminophenyl,
and the salts of these compounds.

Compounds according to embodiment C1 of aspect C to be emphasized are, in one embodiment, those compounds of formula in which
R1, R2, R3, R4 and R5 are hydrogen,
R6 is -T1-Q1, in which T1 is a bond,
either
Q1 is 3-(6-amino-pyridin-3-yl)-phenyl, 4-(6-amino-pyridin-3-yl)-phenyl, 3-(6-methoxy-pyridin-3-yl)-phenyl or 4-(6-methoxy-pyridin-3-yl)-phenyl,
or Q1 is 3-[2-(4-methyl-piperazin-1-yl)-pyridin-4-yl)]-phenyl or 4-[2-(4-methyl-piperazin-1-yl)-pyridin-4-yl)]-phenyl,
or Q1 is 3′-(R61)-1,1′-biphenyl-4-yl or 4′-(R61)-1,1′-biphenyl-4-yl,
or Q1 is 5-[2-(4-methyl-piperazin-1-yl)-pyridin-4-yl)]-thiophen-2-yl,
or Q1 is 5-[4-(R61)-phenyl]-thiophen-2-yl or 5-[3-(R61)-phenyl]-thiophen-2-yl,
or Q1 is 3-(1N-methyl-indol-5-yl)-phenyl, 4-(1N-methyl-indol-5-yl)-phenyl, 3-(1N-methyl-pyrazol-4-yl)-phenyl or 4-(1N-methyl-pyrazol-4-yl)-phenyl,
or Q1 is 5-(1N-methyl-pyrazol-4-yl)-thiophen-2-yl,
or Q1 is 3-(3,5-dimethyl-isoxazol-4-yl)-phenyl or 4-(3,5-dimethyl-isoxazol-4-yl)-phenyl,
in which
R61 is -T2-N(R611)R612, in which
T2 is methylene, dimethylene or trimethylene,
either
R611 and R612 are both methyl,
or
R611 and R612 together and with inclusion of the nitrogen atom, to which they are bonded, form a heterocyclic ring Het1, in which
Het1 is morpholino or 4N-methyl-piperazino,
R7 is hydroxyl,
and the salts of these compounds.

Compounds according to embodiment C1 of aspect C to be emphasized are, in another embodiment, those compounds of formula I in which
R1, R2, R3, R4 and R5 are hydrogen,
R6 is -T1-Q1, in which T1 is a bond,
either
Q1 is 3-(6-amino-pyridin-3-yl)-phenyl, 4-(6-amino-pyridin-3-yl)-phenyl, 3-(6-methoxy-pyridin-3-yl)-phenyl or 4-(6-methoxy-pyridin-3-yl)-phenyl,
or Q1 is 3-[2-(4-methyl-piperazin-1-yl)-pyridin-4-yl)]-phenyl or 4-[2-(4-methyl-piperazin-1-yl)-pyridin-4-yl)]-phenyl,
or Q1 is 3′-(R61)-1,1′-biphenyl-4-yl or 4′-(R61)-1,1′-biphenyl-4-yl,
or Q1 is 5-[2-(4-methyl-piperazin-1-yl)-pyridin-4-yl)]-thiophen-2-yl,
or Q1 is 5-[4-(R61)-phenyl]-thiophen-2-yl or 5-[3-(R61)-phenyl]-thiophen-2-yl,
or Q1 is 3-(1N-methyl-indol-5-yl)-phenyl, 4-(1N-methyl-indol-5-yl)-phenyl, 3-(1N-methyl-pyrazol-4-yl)-phenyl or 4-(1N-methyl-pyrazol-4-yl)-phenyl,
or Q1 is 5-(1N-methyl-pyrazol-4-yl)-thiophen-2-yl,
or Q1 is 3-(3,5-dimethyl-isoxazol-4-yl)-phenyl or 4-(3,5-dimethyl-isoxazol-4-yl)-phenyl,
in which
R61 is -T2-N(R611)R612, in which T2 is methylene, dimethylene or trimethylene,
either
R611 and R612 are both methyl,
or
R611 and R612 together and with inclusion of the nitrogen atom, to which they are bonded, form a heterocyclic ring Het1, in which
Het1 is morpholino or 4N-methyl-piperazino,
R7 is 2-aminophenyl,
and the salts of these compounds.

Compounds according to embodiment C2 of aspect C to be emphasized are those compounds of formula I in which
R1, R2, R3, R4 and R5 are hydrogen,
R6 is -T1-Q1, in which T1 is a bond,
either
Q1 is 3-(6-amino-pyridin-3-yl)-phenyl, 4-(6-amino-pyridin-3-yl)-phenyl, 3-(6-methoxy-pyridin-3-yl)-phenyl or 4-(6-methoxy-pyridin-3-yl)-phenyl,
or Q1 is 3′-(R61)-1,1′-biphenyl-4-yl or 4′-(R61)-1,1′-biphenyl-4-yl,
or Q1 is 3-(1N-methyl-indol-5-yl)-phenyl, 4-(1N-methyl-indol-5-yl)-phenyl, 3-(1N-methyl-pyrazol-4-yl)-phenyl or 4-(1N-methyl-pyrazol-4-yl)-phenyl,
in which
R61 is -T2-N(R611)R612, in which T2 is 1-2C-alkylene,
R611 and R612 together and with inclusion of the nitrogen atom, to which they are bonded, form a heterocyclic ring Het1, in which Het1 is morpholino,
R7 is hydroxyl, or 2-aminophenyl,
and the salts of these compounds.

Compounds according to embodiment C1 of aspect C to be more emphasized are, in one embodiment, those compounds of formula I in which
R1, R2, R3, R4 and R5 are hydrogen,
R6 is -T1-Q1, in which T1 is a bond,
Q1 is any one selected from 3-(6-amino-pyridin-3-yl)-phenyl, 4-(6-amino-pyridin-3-yl)-phenyl, 3-(6-methoxy-pyridin-3-yl)-phenyl, 4-(6-methoxy-pyridin-3-yl)-phenyl, 3-[2-(4-methyl-piperazin-1-yl)-pyridin-4-yl)]-phenyl, 4-[2-(4-methyl-piperazin-1-yl)-pyridin-4-yl)]-phenyl, 3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 4′-(3-morpholin-4-yl-propyl)-biphenyl-3-yl, 4′-(4-methyl-piperazin-1-yl-methyl)-biphenyl-3-yl, 2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 5-[2-(4-methyl-piperazin-1-yl)-pyridin-4-yl)]-thiophen-2-yl, 5-[4-(2-morpholin-4-yl-ethyl)-phenyl]-thiophen-2-yl, 5-[4-(morpholin-4-yl-methyl)-phenyl]-thiophen-2-yl, 5-[3-(morpholin-4-yl-methyl)-phenyl]-thiophen-2-yl, 4-(1N-methyl-indol-5-yl)-phenyl, 3-(1N-methyl-pyrazol-4-yl)-phenyl, 4-(1N-methyl-pyrazol-4-yl)-phenyl, 5-(4-dimethylaminomethyl-phenyl)-thiophen-2-yl, 5-(1N-methyl-pyrazol-4-yl)-thiophen-2-yl, and 4-(3,5-dimethyl-isoxazol-4-yl)-phenyl,
R7 is hydroxyl,
and the salts of these compounds.

Compounds according to embodiment C1 of aspect C to be more emphasized are, in another embodiment, those compounds of formula I in which
R1, R2, R3, R4 and R5 are hydrogen,
R6 is -T1-Q1, in which T1 is a bond,
Q1 is any one selected from 3-(6-amino-pyridin-3-yl)-phenyl, 4-(6-amino-pyridin-3-yl)-phenyl, 3-(6-methoxy-pyridin-3-yl)-phenyl, 4-(6-methoxy-pyridin-3-yl)-phenyl, 3-[2-(4-methyl-piperazin-1-yl)-pyridin-4-yl)]-phenyl, 4-[2-(4-methyl-piperazin-1-yl)-pyridin-4-yl)]-phenyl, 3′-(2-morpholin-4-yl-ethyl)-biphenyl-4-yl, 3′-(2-morpholin-4-yl-ethyl)-biphenyl-3-yl, 4′-(2-morpholin-4-yl-ethyl)-biphenyl-4-yl, 4′-(2-morpholin-4-yl-ethyl)-biphenyl-3-yl, 3′-(morpholin-4-yl-methyl)-biphenyl-3-yl, 4′-(morpholin-4-yl-methyl)-biphenyl-3-yl, 4′-(3-morpholin-4-yl-propyl)-biphenyl-3-yl, 4′-(4-methyl-piperazin-1-yl-methyl)-biphenyl-3-yl, 2′-dimethylaminomethyl-biphenyl-4-yl, 4′-dimethylaminomethyl-biphenyl-4-yl, 2′-dimethylaminomethyl-biphenyl-3-yl, 4′-dimethylaminomethyl-biphenyl-3-yl, 5-[2-(4-methyl-piperazin-1-yl)-pyridin-4-yl)]-thiophen-2-yl,
   5-[4-(2-morpholin-4-yl-ethyl)-phenyl]-thiophen-2-yl, 5-[4-(morpholin-4-yl-methyl)-phenyl]-thiophen-2-yl, 5-[3-(morpholin-4-yl-methyl)-phenyl]-thiophen-2-yl, 4-(1N-methyl-indol-5-yl)-phenyl, 3-(1N-methyl-pyrazol-4-yl)-phenyl, 4-(1N-methyl-pyrazol-4-yl)-phenyl, 5-(4-dimethylaminomethyl-phenyl)-thiophen-2-yl, 5-(1N-methyl-pyrazol-4-yl)-thiophen-2-yl, and
   4-(3,5-dimethyl-isoxazol-4-yl)-phenyl,
R7 is 2-aminophenyl,
and the salts of these compounds.

Compounds according to embodiment C1 of aspect C to be in particular emphasized are, in one embodiment, those compounds of formula I in which
R1, R2, R3, R4 and R5 are hydrogen,
R6 is -T1-Q1, in which T1 is a bond,
Q1 is any one selected from 4-(6-amino-pyridin-3-yl)-phenyl, 4-[2-(4-methyl-piperazin-1-yl)-pyridin-4-yl)]-phenyl, 3-[2-(4-methyl-piperazin-1-yl)-pyridin-4-yl)]-phenyl, 4′-(2-morpholin-4-yl-ethyl)-biphenyl-3-yl, 4′-dimethylaminomethyl-biphenyl-4-yl, 5-[2-(4-methyl-piperazin-1-yl)-pyridin-4-yl)]-thiophen-2-yl, 4-(1N-methyl-pyrazol-4-yl)-phenyl, and 5-(4-dimethylaminomethyl-phenyl)-thiophen-2-yl,
R7 is hydroxyl,
and the salts of these compounds.

Compounds according to embodiment C1 of aspect C to be in particular emphasized are, in another embodiment, those compounds of formula I in which
R1, R2, R3, R4 and R5 are hydrogen,
R6 is -T1-Q1, in which T1 is a bond,
Q1 is any one selected from 4-(6-amino-pyridin-3-yl)-phenyl, 4-[2-(4-methyl-piperazin-1-yl)-pyridin-4-yl)]-phenyl, 3-[2-(4-methyl-piperazin-1-yl)-pyridin-4-yl)]-phenyl, 4′-(2-morpholin-4-yl-ethyl)-biphenyl-3-yl, 4′-dimethylaminomethyl-biphenyl-4-yl, 5-[2-(4-methyl-piperazin-1-yl)-pyridin-4-yl)]-thiophen-2-yl, 4-(1N-methyl-pyrazol-4-yl)-phenyl, and 5-(4-dimethylaminomethyl-phenyl)-thiophen-2-yl,
R7 is 2-aminophenyl,
and the salts of these compounds.

A special interest in the compounds according to the present invention refers to those compounds of formula (I) which are included—within the scope of this invention—by one or, when possible, a combination of more of the following embodiments:

In an embodiment of the compounds of formula I R1, R2, R3, R4 and R5 are all hydrogen.

In a further embodiment of the compounds of formula I, R7 is hydroxyl. A further embodiment of the compounds according to the present invention relates to those compounds of formula I, in which R7 is 2-aminophenyl. A further embodiment of the compounds according to the present invention relates to those compounds of formula I, in which R7 is aminopyridyl. A further embodiment of the compounds according to the present invention relates to those compounds of formula I, in which R7 is Cycl, whereby in a subembodiment thereof Cycl is 2-phenyl.

In a further embodiment of the compounds of formula I, T1 is a bond.

In further embodiments of the compounds of formula I R6 is substituted by R61, and is Aa1, Ha1 or Ha2. A further embodiment of the compounds according to the present invention relates to those compounds of formula I, in which R6 is substituted by R61, and is Ah1 or Hh1. A further embodiment of the compounds according to the present invention relates to those compounds of formula I, in which R6 is substituted by R61, and is Ha3. A further embodiment of the compounds according to the present invention relates to those compounds of formula I, in which R6 is 3-(pyridinyl)-phenyl or 4-(pyridinyl)-phenyl, each of which is substituted by R61 on the pyridinyl moiety. A further embodiment of the compounds according to the present invention relates to those compounds of formula I, in which R6 is 3-(pyridin-3-yl)-phenyl, 3-(pyridin-4-yl)-phenyl, 4-(pyridin-3-yl)-phenyl, or 4-(pyridin-4-yl)-phenyl, each of which is substituted by R61 on the pyridinyl moiety. A further embodiment of the compounds according to the present invention relates to those compounds of formula I, in which R6 is 3-(pyridin-3-yl)-phenyl or 4-(pyridin-3-yl)-phenyl, each of which is substituted by R61 on the pyridinyl moiety. A further embodiment of the compounds according to the present invention relates to those compounds of formula I, in which R6 is 3-[6-(R61)-pyridin-3-yl]-phenyl or 4-[6-(R61)-pyridin-3-yl]-phenyl. A further embodiment of the compounds according to the present invention relates to those compounds of formula I, in which R6 is 3-(pyridin-4-yl)-phenyl or 4-(pyridin-4-yl)-phenyl, each of which is substituted by R61 on the pyridinyl moiety. A further embodiment of the compounds according to the present invention relates to those compounds of formula I, in which R6 is 3-[2-(R61)-pyridin-4-yl]-phenyl or 4-[2-(R61)-pyridin-4-yl]-phenyl. A further embodiment of the compounds according to the present invention relates to those compounds of formula I, in which R6 is 1,1′-biphenyl-4-yl or 1,1′-biphenyl-3-yl, each of which is substituted by R61 on the terminal phenyl moiety. A further embodiment of the compounds according to the present invention relates to those compounds of formula I, in which R6 is 3′-(R61)-1,1′-biphenyl-4-yl, 4′-(R61)-1,1′-biphenyl-4-yl, 3′-(R61)-1,1′-biphenyl-3-yl or 4′- (R61)-1,1′-biphenyl-3-yl. A further embodiment of the compounds according to the present invention relates to those compounds of formula I, in which R6 is 3′-(R61)-1,1′-biphenyl-4-yl or 4′-(R61)-1,1′-biphenyl-4-yl. A further embodiment of the compounds according to the present invention relates to those compounds of formula I, in which R6 is 3′-(R61)-1,1′-biphenyl-3-yl or 4′-(R61)-1,1′-biphenyl-3-yl. A further embodiment of the compounds according to the present invention relates to those compounds of formula I, in which R6 is 4′-(R61)-1,1′-biphenyl-3-yl or 4′-(R61)-1,1′-biphenyl-4-yl. A further embodiment of the compounds according to the present invention relates to those compounds of formula I, in which R6 is pyridinyl-thiophenyl, which is substituted by R61 on the pyridinyl moiety. A further embodiment of the compounds according to the present invention relates to those compounds of formula I, in which R6 is [2-(R61)-pyridin-4-yl]-thiophenyl, such as e.g. 5-[2-(R61)-pyridin-4-yl]-thiophen-2-yl. A further embodiment of the compounds according to the present invention relates to those compounds of formula I, in which R6 is [6-(R61)-pyridin-3-yl]-thiophenyl, such as e.g. 5-[6-(R61)-pyridin-3-yl]-thiophen-2-yl. A further embodiment of the compounds according to the present invention relates to those compounds of formula I, in which R6 is bipyridyl, which is substituted by R61 on the terminal pyridinyl moiety. A further embodiment of the compounds according to the present invention relates to those compounds of formula I, in which R6 is [2-(R61)-pyridin-4-yl]-pyridinyl, such as e.g. 2-[2-(R61)-pyridin-4-yl]-pyridin-4-yl or 6-[2-(R61)-pyridin-4-yl]-pyridin-3-yl. A further embodiment of the compounds according to the present invention relates to those compounds of formula I, in which R6 is [6-(R61)-pyridin-3-yl]-pyridinyl, such as e.g. 2-[6-(R61)-pyridin-3-yl]-pyridin-4-yl or 6-[6-(R61)-pyridin-3-yl]-pyridin-3-yl. A further embodiment of the compounds according to the present invention relates to those compounds of formula I, in which R6 is phenyl-thiophenyl, which is substituted by R61 on the phenyl moiety. A further embodiment of the compounds according to the present invention relates to those compounds of formula I, in which R6 is [3-(R61)-phenyl]-thiophenyl, such as e.g. 5-[3-(R61)-phenyl]-thiophen-2-yl. A further embodiment of the compounds according to the present invention relates to those compounds of formula I, in which R6 is [4-(R61)-phenyl]-thiophenyl, such as e.g. 5-[4-(R61)-phenyl]-thiophen-2-yl. A further embodiment of the compounds according to the present invention relates to those compounds of formula I, in which R6 is phenyl-pyridinyl, which is substituted by R61 on the phenyl moiety. A further embodiment of the compounds according to the present invention relates to those compounds of formula I, in which R6 is [3-(R61)-phenyl]-pyridinyl, such as e.g. 2-[3-(R61)-phenyl]-pyridin-4-yl or 6-[3-(R61)-phenyl]-pyridin-3-yl. A further embodiment of the compounds according to the present invention relates to those compounds of formula I, in which R6 is [4-(R61)-phenyl]-pyridinyl, such as e.g. 2-[4-(R61)-phenyl]-pyridin-4-yl or 6-[4-(R61)-phenyl]-pyridin-3-yl. A further embodiment of the compounds according to the present invention relates to those compounds of formula I, in which R6 is [1N-(1-4C-alkyl)-indolyl]-phenyl or [1N-(1-4C-alkyl)-pyrazolyl]-phenyl. A further embodiment of the compounds according to the present invention relates to those compounds of formula I, in which R6 is [1N-(1-2C-alkyl)-indol-5-yl]-phenyl or [1N-(1-2C-alkyl)-pyrazol-4-yl]-phenyl. A further embodiment of the compounds according to the present invention relates to those compounds of formula I, in which R6 is 3-(1N-methyl-pyrazol-4-yl)-phenyl or 4-(1N-methyl-pyrazol-4-yl)-phenyl. A further embodiment of the compounds according to the present invention relates to those compounds of formula I, in which R6 is [1N-(1-2C-alkyl)-pyrazol-4-yl]-pyridinyl, such as e.g. 2-(1N-methyl-pyrazol-4-yl)-pyridin-4-yl or 6-(1N-methyl-pyrazol-4-yl)-pyridin-3-yl. A further embodiment of the compounds according to the present invention relates to those compounds of formula I, in which R6 is triazolyl-phenyl, which is substituted by R61 on the triazolyl moiety. A further embodiment of the compounds according to the present invention relates to those compounds of formula I, in which R6 is {1N—(R61)-[1,2,3]triazol-4-yl}-phenyl, such as e.g. 3-{1N—(R61)-[1,2,3]triazol-4-yl}-phenyl or 4-{1N—(R61)-[1,2,3]triazol-4-yl}-phenyl. A further embodiment of the compounds according to the present invention relates to those compounds of formula I, in which R61 is -T2-N(R611)R612.

In a further embodiment of the compounds of formula I T2 is a bond. A further embodiment of the compounds according to the present invention relates to those compounds of formula I, in which T2 is 1-4C-alkylene, such as e.g. 1-2C-alkylene. A further embodiment of the compounds according to the present invention relates to those compounds of formula I, in which T2 is methylene. A further embodiment of the compounds according to the present invention relates to those compounds of formula I, in which T2 is dimethylene.

A further embodiment of the compounds according to the present invention relates to those compounds of formula I, in which T2 is trimethylene. A further embodiment of the compounds according to the present invention relates to those compounds of formula I, in which R611 and R612 are both hydrogen.

A further embodiment of the compounds according to the present invention relates to those compounds of formula I, in which R611 and R612 are both methyl. A further embodiment of the compounds according to the present invention relates to those compounds of formula I, in which R611 and R612 together and with inclusion of the nitrogen atom, to which they are attached, form a morpholino ring. A further embodiment of the compounds according to the present invention relates to those compounds of formula I, in which R611 and R612 together and with inclusion of the nitrogen atom, to which they are attached, form a 4N-methyl-piperazino ring. A further embodiment of the compounds according to the present invention relates to those compounds of formula I, in which R611 and R612 together and with inclusion of the nitrogen atom, to which they are attached, form a pyrrolidino ring. A further embodiment of the compounds according to the present invention relates to those compounds of formula I, in which R611 and R612 together and with inclusion of the nitrogen atom, to which they are attached, form a piperidino ring. A further embodiment of the compounds according to the present invention relates to those compounds of formula I, in which R61 is —O-T3-N(R613)R614.

A further embodiment of the compounds according to the present invention relates to those compounds of formula I, in which T3 is dimethylene. A further embodiment of the compounds according to the present invention relates to those compounds of formula I, in which T3 is trimethylene.

A further embodiment of the compounds according to the present invention relates to those compounds of formula I, in which R613 and R614 are both methyl. A further embodiment of the compounds according to the present invention relates to those compounds of formula I, in which R613 and R614 together and with inclusion of the nitrogen atom, to which they are attached, form a morpholino ring. A further embodiment of the compounds according to the present invention relates to those compounds of formula I, in which R613 and R614 together and with inclusion of the nitrogen atom, to which they are attached, form a 4N-methyl-piperazino ring. A further embodiment of the compounds according to the present invention relates to those compounds of formula I, in which R613 and R614 together and with inclusion of the nitrogen atom, to which they are attached, form a pyrrolidino ring. A further embodiment of the compounds according to the present invention relates to those compounds of formula I, in which R613 and R614 together and with inclusion of the nitrogen atom, to which they are attached, form a piperidino ring.

A further embodiment of the compounds according to the present invention relates to those compounds of formula I, in which R61 is -T4-Het3, in which
T4 is a bond, methylene, dimethylene or trimethylene, and
Het3 is 1N-methyl-piperidin-4yl.

A further embodiment of the compounds according to the present invention relates to those compounds of formula I, in which R61 is —O-T5-Het4, in which
T5 is a bond, methylene, dimethylene or trimethylene, and
Het4 is 1N-methyl-piperidin-4yl.

A further embodiment of the compounds according to the present invention relates to those compounds of formula I, in which R61 is any one selected from 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl; and R7 is hydroxyl.

A further embodiment of the compounds according to the present invention relates to those compounds of formula I, in which R61 is any one selected from 3-morpholin-4-yl-propyl, 2-morpholin-4-yl-ethyl, morpholin-4-yl-methyl, 3-(4-methyl-piperazin-1-yl)-propyl, 2-(4-methyl-piperazin-1-yl)-ethyl, (4-methyl-piperazin-1-yl)-methyl, 3-pyrrolidin-1-yl-propyl, 2-pyrrolidin-1-yl-ethyl, pyrrolidin-1-yl-methyl, 3-piperidin-1-yl-propyl, 2-piperidin-1-yl-ethyl, piperidin-1-yl-methyl, 3-morpholin-4-yl-propoxy, 2-morpholin-4-yl-ethoxy, 3-pyrrolidin-1-yl-propoxy, 2-pyrrolidin-1-yl-ethoxy, 3-(4-methyl-piperazin-1-yl)-propoxy, 2-(4-methyl-piperazin-1-yl)-ethoxy, 3-(1-methyl-piperidin-4-yl)-propoxy, 2-(1-methyl-piperidin-4-yl)-ethoxy, 3-piperidin-1-yl-propoxy, 2-piperidin-1-yl-ethoxy, dimethylaminomethyl, 2-dimethylamino-ethyl, 3-dimethylamino-propyl, methylsulphonylamino, dimethylsulphamoyl, acetamido, amino, dimethylamino, morpholino, piperidino, pyrrolidino, 4-methyl-piperazino, hydroxy, trifluoromethyl, methoxy, (2-dimethylamino-ethylamino)-carbonyl, (2-methoxy-ethylamino)methyl, aminomethyl, acetylamino-methyl, methylsulphonylamino-methyl, cyclopentylaminomethyl, cyclopropylaminomethyl and hydroxymethyl; and R7 is 2-aminophenyl.

A further embodiment of the compounds according to the present invention relates to those compounds of formula I, in which R6 is 4-(6-amino-pyridin-3-yl)-phenyl. A further embodiment of the compounds according to the present invention relates to those compounds of formula I, in which R6 is 4-[2-(4-methyl-piperazin-1-yl)-pyridin-4-yl)]-phenyl. A further embodiment of the compounds according to the present invention relates to those compounds of formula I, in which R6 is 3-[2-(4-methyl-piperazin-1-yl)-pyridin-4-yl)]-phenyl. A further embodiment of the compounds according to the present invention relates to those compounds of formula I, in which R6 is 4′-(2-morpholin-4-yl-ethyl)-biphenyl-3-yl. A further embodiment of the compounds according to the present invention relates to those compounds of formula I, in which R6 is 4′-dimethylaminomethyl-biphenyl-4-yl. A further embodiment of the compounds according to the present invention relates to those compounds of formula I, in which R6 is 5-[2-(4-methyl-piperazin-1-yl)-pyridin-4-yl)]-thiophen-2-yl. A further embodiment of the compounds according to the present invention relates to those compounds of formula I, in which R6 is 5-(4-dimethylaminomethyl-phenyl)-thiophen-2-yl.

A special embodiment of the compounds according to the present invention relates to those compounds of formula I, in which R1, R2, R3, R4 and R5 are all hydrogen, and R7 is hydroxyl. Another special embodiment of the compounds according to the present invention relates to those compounds of formula I, in which R1, R2, R3, R4 and R5 are all hydrogen, and R7 is 2-aminophenyl. It is to be understood, that the present invention also includes any or all possible combinations and subsets of the embodiments defined herein afore.

Exemplary compounds according to this invention may include any one selected from
1. (E)-N-Hydroxy-3-{1-[4-(1-methyl-1H-indol-5-yl)-benzenesulfonyl]-1H-pyrrol-3-yl}-acrylamide,
2. (E)-N-Hydroxy-3-{1-[4-(1-methyl-1H-pyrazol-4-yl)-benzenesulfonyl]-1H-pyrrol-3-yl}-acrylamide,
3. (E)-N-Hydroxy-3-{1-[4-(6-methoxy-pyridin-3-yl)-benzenesulfonyl]-1H-pyrrol-3-yl}-acrylamide,
4. (E)-3-{1-[4-(6-Amino-pyridin-3-yl)-benzenesulfonyl]-1H-pyrrol-3-yl}-N-hydroxy-acrylamide,
5. (E)-N-(2-Amino-phenyl)-3-{1-[4-(6-methoxy-pyridin-3-yl)-benzenesulfonyl]-1H-pyrrol-3-yl}-acrylamide,
6. (E)-N-(2-Amino-phenyl)-3-{1-[4-(6-amino-pyridin-3-yl)-benzenesulfonyl]-1H-pyrrol-3-yl}-acrylamide,
7. (E)-N-(2-Amino-phenyl)-3-{1-[4-(1-methyl-1H-pyrazol-4-yl)-benzenesulfonyl]-1H-pyrrol-3-yl}-acrylamide,
8. (E)-N-Hydroxy-3-{1-[4′-(2-morpholin-4-yl-ethyl)-biphenyl-4-sulfonyl]-1H-pyrrol-3-yl}-acrylamide,
9. (E)-N-Hydroxy-3-{1-[3′-(2-morpholin-4-yl-ethyl)-biphenyl-4-sulfonyl]-1H-pyrrol-3-yl}-acrylamide,
10. (E)-3-{1-[3-(6-Amino-pyridin-3-yl)-benzenesulfonyl]-1H-pyrrol-3-yl}-N-hydroxy-acrylamide,
11. (E)-N-Hydroxy-3-{1-[3-(6-methoxy-pyridin-3-yl)-benzenesulfonyl]-1H-pyrrol-3-yl}-acrylamide,
12. (E)-N-Hydroxy-3-{1-[3-(1-methyl-1H-pyrazol-4-yl)-benzenesulfonyl]-1H-pyrrol-3-yl}-acrylamide,
13. (E)-N-Hydroxy-3-{1-[3-(1-methyl-1H-indol-5-yl)-benzenesulfonyl]-1H-pyrrol-3-yl}-acrylamide,
14. (E)-N-(2-Amino-phenyl)-3-{1-[3-(6-methoxy-pyridin-3-yl)-benzenesulfonyl]-1H-pyrrol-3-yl}-acrylamide,
15. (E)-N-(2-Amino-phenyl)-3-{1-[3-(1-methyl-1H-pyrazol-4-yl)-benzenesulfonyl]-1H-pyrrol-3-yl}-acrylamide,
16. (E)-N-Hydroxy-3-{1-[4′-(2-morpholin-4-yl-ethyl)-biphenyl-3-sulfonyl]-1H-pyrrol-3-yl}-acrylamide,
17. (E)-N-(2-Amino-phenyl)-3-{1-[3-(6-amino-pyridin-3-yl)-benzenesulfonyl]-1H-pyrrol-3-yl}-acrylamide,
18. (E)-N-(2-Amino-phenyl)-3-{1-[3′-(2-morpholin-4-yl-ethyl)-biphenyl-3-sulfonyl]-1H-pyrrol-3-yl}-acrylamide,
19. (E)-N-(2-Amino-phenyl)-3-{1-[4′-(2-morpholin-4-yl-ethyl)-biphenyl-3-sulfonyl]-1H-pyrrol-3-yl}-acrylamide,
20. (E)-N-Hydroxy-3-{1-[3′-(2-morpholin-4-yl-ethyl)-biphenyl-3-sulfonyl]-1H-pyrrol-3-yl}-acrylamide,
21. (E)-N-Hydroxy-3-[1-(2′-methanesulfonylamino-biphenyl-4-sulfonyl)-1H-pyrrol-3-yl]-acrylamide
22. (E)-N-hydroxy-3-[1-(3′-methanesulfonylamino-biphenyl-4-sulfonyl)-1H-pyrrol-3-yl]-acrylamide
23. (E)-N-Hydroxy-3-[1-(4′-methanesulfonylamino-biphenyl-4-sulfonyl)-1H-pyrrol-3-yl]-acrylamide
24. 4′-[3-((E)-2-Hydroxycarbamoyl-vinyl)-pyrrole-1-sulfonyl]-biphenyl-4-carboxylic acid (2-dimethylamino-ethyl)-amide,
25. 4′-[3-((E)-2-Hydroxycarbamoyl-vinyl)-pyrrole-1-sulfonyl]-biphenyl-3-carboxylic acid (2-dimethylamino-ethyl)-amide,
26. (E)-3-[1-(4′-Dimethylaminomethyl-biphenyl-4-sulfonyl)-1H-pyrrol-3-yl]-N-hydroxy-acrylamide,
27. (E)-3-[1-(2′-Dimethylaminomethyl-biphenyl-4-sulfonyl)-1H-pyrrol-3-yl]-N-hydroxy-acrylamide,
28. (E)-N-Hydroxy-3-(1-{4-[2-(4-methyl-piperazin-1-yl)-pyridin-4-yl]-benzenesulfonyl}-1H-pyrrol-3-yl)-acrylamide,
29. (E)-N-Hydroxy-3-{1-[4′-(toluene-4-sulfonylamino)-biphenyl-4-sulfonyl]-1H-pyrrol-3-yl}-acrylamide,
30. 3′-[3-((E)-2-Hydroxycarbamoyl-vinyl)-pyrrole-1-sulfonyl]-biphenyl-4-carboxylic acid (2-dimethylamino-ethyl)-amide,
31. (E)-N-Hydroxy-3-[1-(3′-morpholin-4-ylmethyl-biphenyl-3-sulfonyl)-1H-pyrrol-3-yl]-acrylamide,
32. (E)-N-Hydroxy-3-(1-{4′-[2-(4-methyl-piperazin-1-yl)-ethoxy]-biphenyl-3-sulfonyl}-1H-pyrrol-3-yl)-acrylamide,
33. (E)-N-Hydroxy-3-(1-{3-[2-(4-methyl-piperazin-1-yl)-pyridin-4-yl]-benzenesulfonyl}-1H-pyrrol-3-yl)-acrylamide,
34. (E)-N-Hydroxy-3-{1-[4′-(2-morpholin-4-yl-ethoxy)-biphenyl-3-sulfonyl]-1H-pyrrol-3-yl}-acrylamide,
35. (E)-N-(2-Amino-phenyl)-3-{1-[4-(1-benzyl-1H-pyrazol-4-yl)-benzenesulfonyl]-1H-pyrrol-3-yl}-acrylamide,
36. (E)-N-Hydroxy-3-[1-(4′-morpholin-4-ylmethyl-biphenyl-3-sulfonyl)-1H-pyrrol-3-yl]-acrylamide,
37. (E)-3-[1-(4′-Dimethylaminomethyl-biphenyl-3-sulfonyl)-1H-pyrrol-3-yl]-N-hydroxy-acrylamide,
38. (E)-N-Hydroxy-3-{1-[4′-(3-morpholin-4-yl-propoxy)-biphenyl-3-sulfonyl]-1H-pyrrol-3-yl}-acrylamide,
39. (E)-N-(2-Amino-phenyl)-3-[1-(4′-dimethylsulfamoyl-biphenyl-4-sulfonyl)-1H-pyrrol-3-yl]-acrylamide,
40. (E)-3-[1-(3′-Acetylamino-biphenyl-4-sulfonyl)-1H-pyrrol-3-yl]-N-(2-amino-phenyl)-acrylamide,
41. (E)-3-[1-(2′-Dimethylaminomethyl-biphenyl-3-sulfonyl)-1H-pyrrol-3-yl]-N-hydroxy-acrylamide,
42. (E)-N-Hydroxy-3-(1-{5-[2-(4-methyl-piperazin-1-yl)-pyridin-4-yl]-thiophene-2-sulfonyl}-1H-pyrrol-3-yl)-acrylamide,
43. (E)-N-Hydroxy-3-{1-[4′-(2-pyrrolidin-1-yl-ethoxy)-biphenyl-3-sulfonyl]-1H-pyrrol-3-yl}-acrylamide,
44. 4′-{3-[(E)-2-(2-Amino-phenylcarbamoyl)-vinyl]-pyrrole-1-sulfonyl}-biphenyl-3-carboxylic acid (2-dimethylamino-ethyl)-amide,
45. (E)-N-Hydroxy-3-{1-[4′-(3-morpholin-4-yl-propyl)-biphenyl-3-sulfonyl]-1H-pyrrol-3-yl}-acrylamide,
46. (E)-3-{1-[5-(4-Dimethylaminomethyl-phenyl)-thiophene-2-sulfonyl]-1H-pyrrol-3-yl}-N-hydroxy-acrylamide,
47. (E)-N-(2-Amino-phenyl)-3-[1-(4′-dimethylaminomethyl-biphenyl-4-sulfonyl)-1H-pyrrol-3-yl]-acrylamide,
48. (E)-N-(2-Amino-phenyl)-3-(1-{4-[2-(4-methyl-piperazin-1-yl)-pyridin-4-yl]-benzenesulfonyl}-1H-pyrrol-3-yl)-acrylamide,
49. (E)-3-[1-(4′-Acetylamino-biphenyl-4-sulfonyl)-1H-pyrrol-3-yl]-N-(2-amino-phenyl)-acrylamide,
50. (E)-N-Hydroxy-3-{1-[5-(3-morpholin-4-ylmethyl-phenyl)-thiophene-2-sulfonyl]-1H-pyrrol-3-yl}-acrylamide,
51. (E)-N-(2-Amino-phenyl)-3-[1-(3′-hydroxymethyl-biphenyl-4-sulfonyl)-1H-pyrrol-3-yl]-acrylamide,
52. (E)-N-(2-Amino-phenyl)-3-{1-[4-(3,5-dimethyl-isoxazol-4-yl)-benzenesulfonyl]-1H-pyrrol-3-yl}-acrylamide,
53. (E)-N-(2-Amino-phenyl)-3-[1-(4′-methanesulfonylamino-biphenyl-4-sulfonyl)-1H-pyrrol-3-yl]-acrylamide,
54. (E)-N-Hydroxy-3-{1-[5-(4-morpholin-4-ylmethyl-phenyl)-thiophene-2-sulfonyl]-1H-pyrrol-3-yl}-acrylamide,
55. (E)-N-Hydroxy-3-[1-(5-{4-[2-(4-methyl-piperazin-1-yl)-ethoxy]-phenyl}-thiophene-2-sulfonyl)-1H-pyrrol-3-yl]-acrylamide,
56. (E)-N-Hydroxy-3-(1-{5-[4-(2-morpholin-4-yl-ethoxy)-phenyl]-thiophene-2-sulfonyl}-1H-pyrrol-3-yl)-acrylamide,
57. (E)-N-Hydroxy-3-(1-{5-[4-(3-morpholin-4-yl-propoxy)-phenyl]-thiophene-2-sulfonyl}-1H-pyrrol-3-yl)-acrylamide,
58. (E)-N-Hydroxy-3-(1-{4′-[(2-methoxy-ethylamino)-methyl]-biphenyl-3-sulfonyl}-1H-pyrrol-3-yl)-acrylamide,
59. (E)-N-(2-Amino-phenyl)-3-[1-(3′-methanesulfonylamino-biphenyl-4-sulfonyl)-1H-pyrrol-3-yl]-acrylamide,
60. (E)-Hydroxy-3-{1-[5-(1-methyl-1H-pyrazol-4-yl)-thiophene-2-sulfonyl]-1H-pyrrol-3-yl}-acrylamide,
61. (E)-N-Hydroxy-3-(1-{5-[4-(2-morpholin-4-yl-ethyl)-phenyl]-thiophene-2-sulfonyl}-1H-pyrrol-3-yl)-acrylamide,
62. (E)-N-Hydroxy-3-{1-[4′-(4-methyl-piperazin-1-ylmethyl)-biphenyl-3-sulfonyl]-1H-pyrrol-3-yl}-acrylamide, and
63. (E)-3-[1-(4′-Cyclopropylaminomethyl-biphenyl-3-sulfonyl)-1H-pyrrol-3-yl]-N-hydroxy-acrylamide,
and the salts thereof.

Further on, exemplary compounds according to this invention may also include any one selected from
64. (E)-N-Hydroxy-3-[1-(3′-morpholin-4-ylmethyl-biphenyl-4-sulfonyl)-1H-pyrrol-3-yl]-acrylamide,
65. (E)-3-[1-(4-Benzo[1,3]dioxol-5-yl-benzenesulfonyl)-1H-pyrrol-3-yl]-N-hydroxy-acrylamide,
66. (E)-3-[1-(3′-Amino-biphenyl-4-sulfonyl)-1H-pyrrol-3-yl]-N-hydroxy-acrylamide,
67. (E)-N-Hydroxy-3-[1-(4′-hydroxy-biphenyl-4-sulfonyl)-1H-pyrrol-3-yl]-acrylamide,
68. (E)-N-Hydroxy-3-(1-{4′-[2-(l-methyl-piperidin-4-yl)-ethoxy]-biphenyl-4-sulfonyl}-1H-pyrrol-3-yl)-acrylamide,
69. (E)-3-[1-(3′-Dimethylamino-biphenyl-4-sulfonyl)-1H-pyrrol-3-yl]-N-hydroxy-acrylamide,
70. (E)-3-{1-[4-(2,3-Dihydro-benzofuran-5-yl)-benzenesulfonyl]-1H-pyrrol-3-yl}-N-hydroxy-acrylamide,
71. (E)-N-Hydroxy-3-[1-(4′-morpholin-4-yl-biphenyl-4-sulfonyl)-1H-pyrrol-3-yl]-acrylamide,
72. (E)-N-Hydroxy-3-{1-[3′-(3-pyrrolidin-1-yl-propoxy)-biphenyl-4-sulfonyl]-1H-pyrrol-3-yl}-acrylamide,
73. (E)-N-Hydroxy-3-(1-{3′-[3-(4-methyl-piperazin-1-yl)-propoxy]-biphenyl-4-sulfonyl}-1H-pyrrol-3-yl)-acrylamide,
74. (E)-N-Hydroxy-3-{1-[3′-(3-morpholin-4-yl-propoxy)-biphenyl-4-sulfonyl]-1H-pyrrol-3-yl}-acrylamide,
75. (E)-N-Hydroxy-3-[1-(3′-morpholin-4-ylmethyl-biphenyl-4-sulfonyl)-1H-pyrrol-3-yl]-acrylamide,
76. (E)-N-Hydroxy-3-(1-{4′-[2-(4-methyl-piperazin-1-yl)-ethoxy]-biphenyl-4-sulfonyl}-1H-pyrrol-3-yl)-acrylamide,
77. (E)-N-Hydroxy-3-{1-[4′-(2-morpholin-4-yl-ethoxy)-biphenyl-4-sulfonyl]-1H-pyrrol-3-yl}-acrylamide,
78. (E)-N-Hydroxy-3-{1-[4′-(3-morpholin-4-yl-propoxy)-biphenyl-4-sulfonyl]-1H-pyrrol-3-yl}-acrylamide,
79. (E)-N-Hydroxy-3-(1-{4′-[3-(4-methyl-piperazin-1-yl)-propoxy]-biphenyl-4-sulfonyl}-1H-pyrrol-3-yl)-acrylamide,
80. (E)-N-Hydroxy-3-{1-[3′-(2-pyrrolidin-1-yl-ethoxy)-biphenyl-4-sulfonyl]-1H-pyrrol-3-yl}-acrylamide,
81. (E)-N-Hydroxy-3-{1-[4′-(3-pyrrolidin-1-yl-propoxy)-biphenyl-4-sulfonyl]-1H-pyrrol-3-yl}-acrylamide,
82. (E)-N-Hydroxy-3-[1-(4′-methoxy-biphenyl-4-sulfonyl)-1H-pyrrol-3-yl]-acrylamide,
83. (E)-N-Hydroxy-3-(1-{4-[1-(2-morpholin-4-yl-ethyl)-1H-[1,2,3]triazol-4-yl]-benzenesulfonyl}-1H-pyrrol-3-yl)-acrylamide,
84. (E)-3-[1-(4′-Cyclopentylaminomethyl-biphenyl-4-sulfonyl)-1H-pyrrol-3-yl]-N-hydroxy-acrylamide,
85. (E)-N-Hydroxy-3-[1-(3′-trifluoromethyl-biphenyl-4-sulfonyl)-1H-pyrrol-3-yl]-acrylamide,
86. (E-3-{1-[5-(3-Dimethylaminomethyl-phenyl)-thiophene-2-sulfonyl]-1H-pyrrol-3-yl}-N-hydroxy-acrylamide,
87. (E)-3-[1-(3′-Dimethylaminomethyl-biphenyl-3-sulfonyl)-1H-pyrrol-3-yl]-N-hydroxy-acrylamide,
88. (E)-N-Hydroxy-3-{1-[4′-(2-morpholin-4-yl-ethyl)-biphenyl-3-sulfonyl]-1H-pyrrol-3-yl}-acrylamide,
89. (E)-N-(2-Amino-phenyl)-3-{1-[6-(4-dimethylaminomethyl-phenyl)-pyridine-3-sulfonyl]-1H-pyrrol-3-yl}-acrylamide,
90. (E)-N-Hydroxy-3-{1-[5-(2-methyl-thiazol-4-yl)-thiophene-2-sulfonyl]-1H-pyrrol-3-yl}-acrylamide,
91. (E)-3-[1-(4′-Aminomethyl-biphenyl-3-sulfonyl)-1H-pyrrol-3-yl]-N-hydroxy-acrylamide,
92. (E)-N-Hydroxy-3-(1-{6-[4-(2-pyrrolidin-1-yl-ethoxy)-phenyl]-pyridine-3-sulfonyl}-1H-pyrrol-3-yl)-acrylamide,
93. (E)-3-[1-(4′-Aminomethyl-biphenyl-4-sulfonyl)-1H-pyrrol-3-yl]-N-(2-amino-phenyl)-acrylamide,
94. (E)-3-{1-[5-(3-Aminomethyl-phenyl)-thiophene-2-sulfonyl]-1H-pyrrol-3-yl}-N-hydroxy-acrylamide,
95. (E)-N-(2-Amino-phenyl)-3-{1-[5-(4-dimethylaminomethyl-phenyl)-thiophene-2-sulfonyl]-1H-pyrrol-3-yl}-acrylamide,
96. (E)-N-(2-Amino-phenyl)-3-[1-(3′-dimethylaminomethyl-biphenyl-4-sulfonyl)-1H-pyrrol-3-yl]-acrylamide,
97. (E)-3-{1-[4′-(Acetylamino-methyl)-biphenyl-4-sulfonyl]-1H-pyrrol-3-yl}-N-(2-amino-phenyl)-acrylamide,
98. (E)-N-(2-Amino-phenyl)-3-{1-[4′-(methanesulfonylamino-methyl)-biphenyl-4-sulfonyl]-1H-pyrrol-3-yl}-acrylamide,
99. (E)-N-Hydroxy-3-(1-{5-[4-(2-pyrrolidin-1-yl-ethoxy)-phenyl]-thiophene-2-sulfonyl}-1H-pyrrol-3-yl)-acrylamide,
100. (E)-3-{1-[5-(4-Dimethylsulfamoyl-phenyl)-thiophene-2-sulfonyl]-1H-pyrrol-3-yl}-N-hydroxy-acrylamide,
101. (E)-N-(2-Amino-phenyl)-3-[1-(4′-methanesulfonylamino-biphenyl-3-sulfonyl)-1H-pyrrol-3-yl]-acrylamide,
102. (E)-N-(2-Amino-phenyl)-3-[1-(4′-dimethylaminomethyl-biphenyl-3-sulfonyl)-1H-pyrrol-3-yl]-acrylamide,
103. (E)-N-Hydroxy-3-{1-[2′-(4-methyl-piperazin-1-yl)-[2,4′]bipyridinyl-5-sulfonyl]-1H-pyrrol-3-yl}-acrylamide,
104. (E)-N-(2-Amino-phenyl)-3-{1-[5-(1-methyl-1H-pyrazol-4-yl)-thiophene-2-sulfonyl]-1H-pyrrol-3-yl}-acrylamide,
105. (E)-3-{1-[6-(4-Dimethylaminomethyl-phenyl)-pyridine-3-sulfonyl]-1H-pyrrol-3-yl}-N-hydroxy-acrylamide,
106. (E)-N-(2-Amino-phenyl)-3-(1-{5-[2-(4-methyl-piperazin-1-yl)-pyridin-4-yl]-thiophene-2-sulfonyl}-1H-pyrrol-3-yl)-acrylamide,
107. (E)-N-(2-Amino-phenyl)-3-[1-(4′-morpholin-4-ylmethyl-biphenyl-4-sulfonyl)-1H-pyrrol-3-yl]-acrylamide,
108. (E)-N-(2-Amino-phenyl)-3-{1-[4′-(2-pyrrolidin-1-yl-ethoxy)-biphenyl-4-sulfonyl]-1H-pyrrol-3-yl}-acrylamide,
109. (E)-N-Hydroxy-3-(1-{4-[1-(2-piperidin-1-yl-ethyl)-1H-[1,2,3]triazol-4-yl]-benzenesulfonyl}-1H-pyrrol-3-yl)-acrylamide,
110. (E)-3-[1-(3′-Dimethylaminomethyl-biphenyl-3-sulfonyl)-1H-pyrrol-3-yl]-N-hydroxy-acrylamide,
111. (E)-N-(2-Amino-phenyl)-3-(1-{5-[4-(methynesulfonylamino-methyl)-phenyl]-thiophene-2-sulfonyl}-1H-pyrrol-3-yl)-acrylamide,
112. (E)-N-(2-Amino-phenyl)-3-{1-[3′-(methanesulfonylamino-methyl)-biphenyl-3-sulfonyl]-1H-pyrrol-3-yl}-acrylamide,
113. (E)-3-(1-{5-[4-(Acetylamino-methyl)-phenyl]-thiophene-2-sulfonyl}-1H-pyrrol-3-yl)-N-(2-amino-phenyl)-acrylamide,
114. (E)-N-(2-Amino-phenyl)-3-{1-[5-(3-dimethylaminomethyl-phenyl)-thiophene-2-sulfonyl]-1H-pyrrol-3-yl}-acrylamide,
115. (E)-N-(2-Amino-phenyl)-3-[1-(3′-dimethylaminomethyl-biphenyl-3-sulfonyl)-1H-pyrrol-3-yl]-acrylamide,
116. (E)-3-[1-(3′-Dimethylaminomethyl-biphenyl-4-sulfonyl)-1H-pyrrol-3-yl]-N-hydroxy-acrylamide,
117. (E)-3-{1-[5-(3-Dimethylaminomethyl-phenyl)-thiophene-2-sulfonyl]-1H-pyrrol-3-yl}-N-hydroxy-acrylamide,
118. (E)-3-{1-[3′-(Acetylamino-methyl)-biphenyl-3-sulfonyl]-1H-pyrrol-3-yl}-N-(2-amino-phenyl)-acrylamide,
119. (E)-N-(2-Amino-phenyl)-3-{1-[6-(1-methyl-1H-pyrazol-4-yl)-pyridine-3-sulfonyl]-1H-pyrrol-3-yl}-acrylamide,
120. (E)-N-Hydroxy-3-{1-[6-(1-methyl-1H-pyrazol-4-yl)-pyridine-3-sulfonyl]-1H-pyrrol-3-yl}-acrylamide, and
121. (E)-3-{1-[6-(3-Dimethylaminomethyl-phenyl)-pyridine-3-sulfonyl]-1H-pyrrol-3-yl}-N-hydroxy-acrylamide,
and the salts thereof.

In an embodiment of the foregoing, exemplary compounds according to this invention may especially include any one selected from the group consisting of the compounds 2, 4, 7, 16, 26, 28, 32, 33, 38, 42 and 46 as mentioned afore, and the salts thereof.

As used herein, 4SC-202 and (E)-N-(2-aminophenyl)-3-(1-((4-(1-methyl-1H-pyrazol-4-yl)phenyl)sulfonyl)-1H-pyrrol-3-yl)acrylamide (its chemical name) are used interchangeably and both refer to a compound of the following formula:

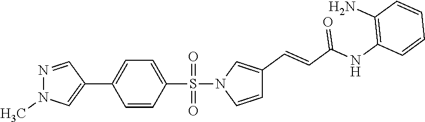 [SMILES: Cn1cc(-c2ccc(S(=O)(=O)n3ccc(/C=C/C(=O)Nc4ccccc4N)c3)cc2)cn1]

Suitable salts for the HDAC inhibitor are acid addition salts or salts with bases. Particular mention may be made of the pharmacologically tolerable inorganic and organic acids and bases customarily used in pharmacy. Those suitable are, on the one hand, water-insoluble and, particularly, water-soluble acid addition salts, the acids being employed in salt preparation in an equimolar quantitative ratio or one differing therefrom, particularly in an equimolar quantitative ratio. On the other hand, salts with bases are—depending on substitution—also suitable, the bases being employed in salt preparation in an equimolar quantitative ratio or one differing therefrom. Pharmacologically intolerable salts, which can be obtained, for example, as process products during the preparation of the HDAC inhibitor on an industrial scale, are converted into pharmacologically tolerable salts by processes known to the person skilled in the art. According to the invention, the HDAC inhibitor as well as its salts may contain, e.g. when isolated in crystalline form, varying amounts of solvents. Included within the scope of the present invention are therefore all solvates and in particular all hydrates of the HDAC inhibitor as well as all solvates and in particular all hydrates of the HDAC inhibitor, in particular such solvates or hydrates comprising about 0.5, 1 or 2 solvate or water molecules per molecule of the HDAC inhibitor or salts thereof.

Particular salts in the context of the present invention are the salts of 4SC-202 with, HBr, methansulfonic acid, hemiethane-1,2-disulfonic acid, benzenesulfonic acid, toluenesulfonic acid and 2-naphthalenesulfonic acid, more particularly toluenesulfonic acid, in particular in a molar ratio of about 1:1.

The HDAC inhibitor and salts thereof can be prepared, for example, as described in detail in WO 2006/097474 A1 and WO 2009/112522 A1, respectively.

### TLR7 Agonists and TLR8 Agonists

The expressions “agonist of TLR7” and “agonist of TLR8” refer to compounds which bind to and activate TLR7 or TLR8 respectively. The terms “activation” and “stimulation” are used indifferently. Those compounds can be natural or synthetic. Many of the compounds that activate TLR7 also activate TLR8. It falls within the ability of the skilled person to determine whether, at a given dose, a compound is specific for one or other of these receptors.

As used herein, the term “at least one TLR7 agonist and/or TLR8 agonist” particularly is to be understood that the pharmaceutical combination product of the present invention may comprise a TLR7 agonist, or a TLR8 agonist, or a TLR7/8 agonist (i.e. a compound acing as an agonist for both TLR7 and TLR8), or both a TLR7 agonist and a TLR8 agonist. In certain embodiments, in regards to the at least one TLR7 agonist and/or TLR8 agonist, the pharmaceutical combination product of the present invention comprises a TLR7 agonist, or a TLR8 agonist, or a TLR7/8 agonist.

Several agonists of TLR7 or TLR8 are known in the art, many of which are envisaged for use in combined therapy with an anti-cancer agent for the treatment of a patient suffering from cancer.

Methods for identifying agonists of TRL7 or agonists of TLR8 are described for example in document WO2004/075865 (3M Innovative Properties Company).

Natural agonists of TLR7 and TLR8 have recently been identified as guanosine- and uridine-rich ssRNA (Diebold, Science 2004, Heil Science, 2004). In addition, synthetic agonists of TLR7 include, but are not limited to: imidazoquinoline-like molecules, imiquimod, resiquimod, gardiquimod, S-27609; and guanosine analogues such as loxoribine (7-allyl-7,8-dihydro-8-oxo-guanosine), 7-Thia-8-oxoguanosine and 7-deazaguanosine, UC-1V150, ANA975 (Anadys Pharmaceuticals), SM-360320 (Sumimoto), 3M-01 and 3M-03 (3M Pharmaceuticals) (see for example Gorden et al., J Immunology, 2005; Schón, Oncogene, 2008; Wu et al., PNAS 2007).

Synthetic agonists of TLR8 include 3M-02 (developed by 3M Pharmaceuticals and commercialised for example by Invivogen as CL075), 3M-03, Poly-G containing 10 guanosine nucleosides connected by phosphothioate linkages (Poly-GlO).

Methods for screening for TLR7 and TRL8 agonists are described in, e.g., US 20060269936, U.S. Pat. No. 7,375,180 and WO2005007672.

In embodiments of the invention, reference may be made to agonists marketed, for example, by the company InvivoGen, i.e. Gardiquimod, Imiquimod and R848 (resiquimod), at least of which may be present in the pharmaceutical combination products of the present invention. In one embodiment, the pharmaceutical combination product comprises at least a compound of formula (I) and Gardiquimod and/or Imiquimod and/or Resiquimod.

In further embodiments, the pharmaceutical combination product comprises a compound of formula (I), which is (E)-N-(2-amino-phenyl)-3-{1-[4-(1-methyl-1H-pyrazol-4-yl)-benzenesulfonyl]-1H-pyrrol-3-yl}-acrylamide or a salt or solvate thereof, preferably the tosylate, and Gardiquimod. In further embodiments, the pharmaceutical combination product comprises a compound of formula (I), which is (E)-N-(2-amino-phenyl)-3-{1-[4-(1-methyl-1H-pyrazol-4-yl)-benzenesulfonyl]-1H-pyrrol-3-yl}-acrylamide or a salt or solvate thereof, preferably the tosylate, and Imiquimod. In further embodiments, the pharmaceutical combination product comprises a compound of formula (I), which is (E)-N-(2-amino-phenyl)-3-{1-[4-(1-methyl-1H-pyrazol-4-yl)-benzenesulfonyl]-1H-pyrrol-3-yl}-acrylamide or a salt or solvate thereof, preferably the tosylate, and Resiquimod. It goes without saying, that the compound of formula (I), which is (E)-N-(2-amino-phenyl)-3-{1-[4-(1-methyl-1H-pyrazol-4-yl)-benzenesulfonyl]-1H-pyrrol-3-yl}-acrylamide or a salt or solvate thereof, preferably the tosylate, may be combined with other components of formula (I) and/or at least one or more of the above-mentioned agonists. Still further, the pharmaceutical combination product according to the present invention may also be combined with other anti-cancer drugs or treatments and with other immunostimulants.

The compounds according to the present invention are to be understood to comprise all tautomeric forms thereof, even if not expressedly shown in the formulae described herein.

The compounds defined herein are to be understood to encompass, where applicable, all stereoismers of said compounds, unless specified otherwise. The term “stereoisomer” as used herein refers to a compound with at least one stereogenic centre, which may be R- or S-configured, as defined by the according IUPAC rules, and encompasses enantiomers and diastereomers as commonly understood by the skilled person. It has to be understood, that in compounds with more than one stereogenic centre, each of the individual stereogenic centres may independently from each other be R- or S-configured. The term “stereoisomer” as used herein also refers to salts of the compounds herein described with optically active acids or bases.

In the present invention, the salts of the compounds according to the present invention are particularly pharmaceutically acceptable salts of the compounds according to the present invention. Pharmaceutically acceptable salts are such salts which are usually considered by the skilled person to be suitable for medical applications, e.g. because they are not harmful to subjects which may be treated with said salts, or which give rise to side effects which are tolerable within the respective treatment. Usually, said pharmaceutically acceptable salts are such salts which are considered as acceptable by the regulatory authorities, such as the US Food and Drug Administration (FDA), the European Medicines Agency (EMA), or the Japanese Ministry of Health, Labor and Welfare Pharmaceuticals and Medical Devices Agency (PMDA). However, the present invention in principle also encompasses salts of the compounds according to the present invention which are as such not pharmaceutically acceptable, e.g. as intermediates in the production of the compounds for use according to the present invention or physiologically functional derivatives thereof, or as intermediates in the production pharmacologically acceptable salts of the compounds used according to the present invention or physiologically functional derivatives thereof.

In each case, the skilled person can readily determine whether a certain compound used according to the present invention or pharmaceutically acceptable derivative thereof can form a salt, i.e. whether said compound according to the present invention or pharmaceutically acceptable derivative thereof has a group which may carry a charge, such as e.g. an amino group, a carboxylic acid group, etc.

Exemplary salts of the compounds of the present invention are acid addition salts or salts with bases, particularly pharmacologically tolerable inorganic and organic acids and bases customarily used in pharmacy, which are either water insoluble or, particularly, water-soluble acid addition salts. Salts with bases may—depending on the substituents of the compounds of the present invention—also be suitable. The tosylate salt is a preferred salt of compounds of formula (I).

Pharmacologically intolerable salts, which can be obtained, for example, as process products during the preparation of the compounds according to the invention on an industrial scale, are also encompassed by the present invention and, if desired, may be converted into pharmacologically tolerable salts by processes known to the person skilled in the art.

According to expert's knowledge the compounds of the invention as well as their salts may contain, e.g. when isolated in crystalline form, varying amounts of solvents. Included within the scope of the invention are therefore solvates and in particular hydrates of the compounds of the present invention as well as solvates and in particular hydrates of the salts and/or physiologically functional derivatives of the compounds of the present invention. More particularly the invention encompasses hydrates of the compounds, salts and/or physiologically functional derivatives according to the present invention, comprising one, two or one half water molecule, with respect to their stoichiometry.

As used herein, the term “room temperature”, “rt” or “r.t.” relates to a temperature of about 25° C., unless specified otherwise.

As used herein, the term “stable” specifies a compound in which the chemical structure is not altered when the compound is stored at a temperature from about −80° C. to about +40° C., particularly from about −80° C. to +25° C. in the absence of light, moisture or other chemically reactive conditions for at least one week, particularly at least one month, more particularly at least six months, even more particularly, at least one year, and/or a compound which under IUPAC standard conditions and in the absence of light, moisture or other chemically reactive conditions maintains its structural integrity long enough to be useful for therapeutic or prophylactic administration to a patient, i.e. at least one week. Compounds which are not stable as described above are particularly not encompassed by the present invention. In particular, such compounds which at IUPAC standard conditions spontaneously decompose within a period of less then one day are regarded as not being stable compounds. The skilled person will readily recognize, based on his general knowledge in his field of expertise, which compounds and which substitution patterns result in stable compounds.

As used herein, the term “treatment” includes complete or partial healing of a disease, prevention of a disease, alleviation of a disease or stop of progression of a given disease.

As used herein, the term “medicament” includes the compounds of formula (I) as described herein, pharmacologically acceptable salts or physiologically functional derivatives thereof, which are to be administered to a subject in pure form, as well as compositions comprising at least one compound according to the present invention, a pharmacologically acceptable salt or physiologically functional derivative thereof, which is suitable for administration to a subject. The medicaments may also comprise at least one of the above described agonists of TLR7 and/or TLR8 in the same or a separately formulated pharmaceutical composition. A medicament is, therefore, a pharmaceutical combination product according to the present invention.

The compounds/agonists used according to the present invention and their pharmacologically acceptable salts and physiologically functional derivatives can be administered to animals, particularly to mammals, and in particular to humans as therapeutics per se, as mixtures with one another or particularly in the form of pharmaceutical preparations or compositions which allow enteral (e.g. oral) or parenteral administration and which comprise as active constituent a therapeutically effective amount of at least one compound according to the present invention, or a salt or physiologically functional derivative thereof, in addition to e.g. one or more components selected from the group comprising customary adjuvants, pharmaceutically innocuous excipients, carriers, buffers, diluents, and/or other customary pharmaceutical auxiliaries.

The pharmaceutical compositions, medical uses and methods of treatment according to the present invention may comprise the more than one compound/agonist according to the present invention.

Pharmaceutical compositions comprising a compound/agonist according to the present invention, or a pharmaceutically acceptable salt or physiologically functional derivative may optionally comprise one or more further therapeutically active substances which are not compounds of formula (I)/agonists according to pharmaceutical combination products of the present invention. As used herein, the term “therapeutically active substance” specifies a substance which upon administration can induce a medical effect in a subject. Said medical effect may include the medical effect described herein for the compounds of formula (I) of the present invention, but may also, in the case of therapeutically active substances which are to be co-administered with the compounds according to the present invention, include other medical substances, such as for example but not exclusively irinotecan, oxaliplatin, capecitabine, 5-fluorouracil, cetuximab (Erbitux), panitumumab (Vectibix), bevacizumab (Avastin), vincristine, vinblastine, vinorelbine, vindesine, taxol, amsacrine, etoposide, etoposide phospahte, Teniposide, actinomycin, anthracyclines, doxorubicin, valrubicin, valrubicin, idarubicin, epirubicin, bleomycin, plicamycin, mitomycin, mechlorethamine, cyclophosphamide, chlorambucil, ifosfamide and other kinase inhibitors.

The term “pharmaceutically acceptable” is well known to the skilled person and particularly means that the respective entity is not harmful to the subject to which the entity or the composition comprising the entity is administered, that said entity is stable and that said entity is chemically compatible (i.e. non-reactive) with other ingredients of the respective pharmaceutical composition.

Medicaments and pharmaceutical compositions according to the present invention, comprising at least one compound of formula (I) and at least one agonist of the above mentioned toll-like receptors according to the present invention or a pharmacologically acceptable salt or a physiologically functional derivative therof include those suitable for oral, rectal, bronchial, nasal, topical, buccal, sub-lingual, vaginal or parenteral (including transdermal, subcutaneous, intramuscular, intrapulmonary, intravascular, intracranial, intraperitoneal, intravenous, intraarterial, intracerebral, intraocular injection or infusion) administration, or those in a form suitable for administration by inhalation or insufflation, including powders and liquid aerosol administration, or by controlled release (e.g. sustained release, pH-controlled release, delayed, release, repeat action release, prolonged release, extended release) systems. Suitable examples of controlled release systems include semipermeable matrices of solid hydrophobic polymers containing the compound of the invention, which matrices may be in form of shaped articles, e.g. films or microcapsules or colloidal drug carriers, e.g., polymeric nanoparticles, or controlled release solid dosage forms, e.g. core tablets or multi-layer tablets.

The production of medicaments or pharmaceutical compositions comprising the compounds/agonists used according to the present invention and their application can be performed according to methods which are well-known to the medical practitioner.

Pharmaceutically acceptable carriers used in the preparation of a pharmaceutical composition or medicament comprising a compound/agonist used according to the present invention, a pharmacologically acceptable salt or physiologically functional derivative thereof, can be either solid or liquid. Solid form pharmaceutical compositions comprising a compound according to the present invention, a pharmacologically acceptable salt or physiologically functional derivative thereof, include powders, tablets, pills, capsules, sachets, suppositories, and dispersible granules. A solid carrier may comprise one or more components, which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component. In tablets, the active component/agonist is mixed with the carrier having the necessary binding capacity in suitable proportions and compacted in the shape and size desired. The tabletting mixture can be granulated, sieved and compressed or direct compressed. Suitable carriers are magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of the active compound with encapsulating material as carrier providing a capsule in which the active component/agonist, with or without carriers, is surrounded by a carrier, which is thus in association with it. Similarly, sachets and lozenges are included. Tablets, powders, capsules, pills, sachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into conveniently sized moulds, allowed to cool, and thereby to solidify. Compositions suitable for vaginal administration may be presented as peccaries, tampons, creams, gels, pastes, foams or sprays containing in addition to the active ingredient such carriers as are known in the art to be appropriate. Liquid preparations include solutions, suspensions, and emulsions, for example, water or water-propylene glycol solutions. For example, parenteral injection liquid preparations can be formulated as solutions in aqueous polyethylene glycol solution.

The compounds/agonists used according to the present invention may be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilizing and/or dispersing agents. Alternatively, the active ingredients may be in powder form, obtained by aseptic isolation of sterile solid or by lyophilization from solution, for re-constitution with a suitable vehicle, e.g. sterile, pyrogen-free water, before use.

Aqueous solutions suitable for oral administration can be prepared by dissolving the active component/agonist used according to the present invention in water and adding for example suitable colorants, flavours, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, which are intended to be converted, shortly before administration, to liquid form preparations for oral administration. Such liquid forms include solutions, suspensions, and emulsions. These preparations may contain, in addition to the active component, for example colorants, flavours, stabilizers, buffers, artificial and natural sweeteners, dispersants, thickeners, solubilizing agents, and the like.

In an embodiment of the present invention the medicament is applied topically, e.g. in the form of transdermal therapeutic systems (e.g. patches) or topical formulations (e.g. liposomes, cremes, ointment, lotion, gels, dispersion, suspension, spray, solution, foam, powder). This may be suitable to reduce possible side effects and, where appropriate, limit the necessary treatment to those areas affected.

Particularly the medicament may comprise carrier materials or excipients, including but not limited to a lipophilic phase (as for example Vaseline, paraffines, triglycerides, waxes, polyalcylsiloxanes), oils (olive oil, peanut oil, castor oil, triglyceride oil), emulsifier (as for example lecithin, phosphatidylglyceroles, alkyl alcohols, sodium lauryl sulfate, polysorbats, Cholesterol, sorbitan fatty acid ester, polyoxyethylene fatty acid glycerol and -ester, poloxamers), preservatives (for instance benzalkonium chloride, chlorobutanol, parabene or thiomersal), flavouring agents, buffer substances (for example salts of acetic acid, citric acid, boric acid, phosphoric acid, tatric acid, trometamole or trolamine), solvents (for instance polyethylenglycols, glycerol, ethanol, isopropanol or propyleneglycol) or solubilizers, agents for achieving a depot effect, salts for modifying the osmotic pressure, carrier materials for patches (for instance polypropylene, ethylene-vinylacetat-copolymer, polyacrylates, silicon) or antioxidants (for example ascorbate, tocopherol, butylhydroxyanisole, gallic acid esters or butylhydroxytoluol).

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatine and glycerine or sucrose and acacia; and mouthwashes comprising the active ingredient in a suitable liquid carrier.

Solutions or suspensions may be applied directly to the nasal cavity by conventional means, for example with a dropper, pipette or spray. The compositions may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant such as a chlorofluorocarbon (CFC), for example dichlorodifluoromethane, trichlorofluoro methane, or dichlorotetrafluoroethane, carbon dioxide, or other suitable gas. The aerosol may conveniently also contain a surfactant such as lecithin. The dose of drug may be controlled by provision of a metered valve.

Alternatively the medicament may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form, for example in capsules or cartridges of, e.g., gelatine, or blister packs from which the powder may be administered by means of an inhaler.

In compositions for administration to the respiratory tract, including intranasal compositions, the compound/agonist used will generally have a small particle size for example of the order of 5 microns or less. Such a particle size may be obtained by means known in the art, for example by micronization.

When desired, compositions adapted to give sustained release of the one or more active ingredients may be employed.

The pharmaceutical preparations are particularly in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, sachet, or lozenge itself, or it can be the appropriate number of any of these in packaged form. Tablets or capsules for oral administration and liquids for intravenous administration and continuous infusion are particular compositions.

Further details on techniques for formulation and administration may be found in the 21^{st} edition of Remington's Pharmaceutical Sciences (Maack Publishing Co. Easton, Pa.).

The compounds/agonists used of the present invention may be used in combination with radiation therapy, or in combination with radiation therapy and other active compounds, already known for the treatment of the medical conditions disclosed herein, whereby a favourable additive or amplifying effect is noticed.

To prepare the pharmaceutical preparations, pharmaceutically inert inorganic or organic excipients can be used. To prepare pills, tablets, coated tablets and hard gelatine capsules, for example, lactose, cornstarch or derivatives thereof, talc, stearic acid or its salts, etc. can be used. Excipients for soft gelatine capsules and suppositories are, for example, fats, waxes, semi-solid and liquid polyols, natural or hardened oils etc. Suitable excipients for the production of solutions and syrups are, for example, water, sucrose, invert sugar, glucose, polyols etc. Suitable excipients for the production of injection solutions are, for example, water, alcohols, glycerol, polyols or vegetable oils.

The dose can vary within wide limits and is to be suited to the individual conditions in each individual case. For the above uses the appropriate dosage will vary depending on the mode of administration, the particular condition to be treated and the effect desired. In general, however, satisfactory results are achieved at dosage rates of about 1 to 100 mg/kg animal body weight particularly 1 to 50 mg/kg of a compound of formula (I). Suitable dosage rates for larger mammals, for example humans, are of the order of from about 10 mg to 3 g/day, conveniently administered once, in divided doses 2 to 4 times a day, or in sustained release form. The agonists according used in the combination product are administered at thereapeutically effective doses, e.g. at concentrations of 100 μM to 1 nM, e.g. at 50 μM to 5 nM, particularly at 30 μM to 10 nM.

The compounds/agonists used according to the present invention are suitable for the treatment of hyperproliferative diseases, such as benign and malignant forms of neoplasia, including cancer.

Exemplary types of cancer in the comtext of the present invention are hepatocarcinoma, adrenocortical carcinoma, AIDS-related cancers including AIDS-related lymphoma, anal cancer, basal cell carcinoma, bile duct cancer, bone cancer, brain tumors including brain stem glioma, cerebellar astrocytoma, cerebral astrocytoma, malignant glioma, ependymoma, medulloblastoma, supratentorial primitive neuroectodermal tumors, visual pathway and hypothalamic glioma, breast cancer, bronchial adenomas/carcinoids, Burkitt's lymphoma, gastrointestinal, carcinoma of unknown primary site, central nervous system lymphoma, cervical cancer, chronic myeloproliferative disorders, colon cancer, colorectal cancer, cutaneous T-cell lymphoma, endometrial cancer, ependymoma, esophageal cancer, extracranial germ cell tumor, extragonadal germ cell tumor, ovarian germ cell tumor, eye cancer including intraocular melanoma and retinoblastoma, gallbladder cancer, gastrointestinal carcinoid tumor, gestational trophoblastic tumor, glioma, childhood brain stem glioma, head and neck cancer, hematologic cancer, adult and childhood (primary) hepatocellular cancer, hypopharyngeal cancer, islet cell or pancreatic cancer, renal cancer, laryngeal cancer, acute lymphoblastic leukemia, adult and childhood acute myeloid leukemia, chronic lymphocytic leukemia, chronic myelogenous leukemia, hairy cell leukemia, lip and oral cavity cancer, liver cancer, lung cancer, including non-small cell lung cancer and small cell lung cancer, Hodgkin's lymphoma, non-Hodgkin's lymphoma, primary central nervous system lymphoma, Waldenstrom's macroglobulinemia, merkel cell carcinoma, mesothelioma, metastatic squamous neck cancer with occult primary site, multiple endocrine neoplasia syndrome, multiple myeloma/plasma cell neoplasm, mycosis fungoides, myelodysplastic syndromes, myelodysplastic myeloproliferative diseases, multiple myeloma, chronic myeloproliferative disorders, nasal cavity and paranasal sinus cancer, nasopharyngeal cancer, neuroblastoma, oral cancer, oropharyngeal cancer, osteosarcoma/malignant fibrous histiocytoma of bone, ovarian cancer, ovarian epithelial cancer, ovarian low malignant potential tumor, pancreatic cancer, parathyroid cancer, penile cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter cancer, transitional cell cancer, rhabdomyosarcoma, salivary gland cancer, Ewing's sarcoma, Kaposi's sarcoma, soft tissue sarcoma, uterine sarcoma, sezary syndrome, skin cancer, including melanoma and non-melanoma skin cancer, small intestine cancer, squamous cell carcinoma, gastric cancer, supratentorial primitive neuroectodermal tumors, testicular cancer, thymoma, thymoma and thymic carcinoma, thyroid cancer, trophoblastic tumor, gestational, endometrial uterine cancer, uterine sarcoma, vaginal cancer, vulvar cancer, Waldenstrom's macroglobulinemia, Wilms' tumor.

In a more particular embodiment of the present invention, the pharmaceutical combination products comprising the herein described compounds/agonists may be used in the treatment of the following cancer types: Prostate, bladder, kidney (i.e. renal), muscle, ovary, skin, lung, pancreas, breast, cervix, colon, liver, connective tissue, placenta, bone, brain, uterus, salivary gland, or testes.

Further details and embodiments of the present invention concerning the agonists relate to compounds disclosed in patent EP 2386557 B1, the contents of which are incorporated herein by reference in their entirety.

Unless specified otherwise, references to the compounds according to the present invention include the pharmaceutically acceptable derivatives, solvates or salts thereof as described herein, as well as to salts of said pharmaceutically acceptable derivatives, solvates of salts and pharmaceutically acceptable derivatives, and optionally solvates of salts of pharmaceutically acceptable derivatives. As used herein, the term “pharmaceutically acceptable derivative” is for instance a prodrug of the compound according to formula (I) or of the at least one TLR7 agonist and/or TLR8 agonist.

In an embodiment of the invention, the at least one TLR7 agonist and/or TLR8 agonist in the pharmaceutical combination product comprising furthermore a compound according to formula (I) is used in combination with chemotherapy, i.e. in combination with an anti-cancer agent. In a preferred embodiment, the anti-cancer agent is selected from taxol; taxotere; platinum complexes such as cisplatin, carboplatin and oxaliplatin; doxorubicin; taxanes such as docetaxel and paclitaxel; vinca alkaloids such as vinblastine, vincristine and vinorelbine; genistein; erbstatin; and lavendustin. The present invention relates to a method for treating a cancer patient comprising administering a therapeutically effective amount of a at least one TLR7 agonist and/or TLR8 antagonist, wherein said patient expresses TLR7 and/or TLR8 respectively in cancer cells.

In other terms, the invention relates to a pharmaceutical combination product as defined herein for use in the treatment of cancer as defined above for the manufacture of a medicament for treating cancer in a patient wherein said patient expresses TLR7 and/or TRL8 respectively in cancer cells.

The invention also relates to a pharmaceutical combination product as defined herein for use in treating cancer in a patient, wherein said patient expresses TLR7 in cancer cells.

The invention also relates to a pharmaceutical combination product as defined herein for use in a patient wherein said patient expresses TLR8 in cancer cells.

Methods for determining that whether the patient expresses TLR7 or TLR8 in cancer cells or not are know in the art and comprise methods, wherein the cells of the patient may be identified by the use of agents specifically binding to the receptor's and wherein these agents carry detectable moieties, for example fluorescence-labelled antibodies and the like.

The following examples are only illustrative and should not be considered as limiting the scope and disclosure of the present invention. The person skilled in the art is aware that modifications to the below described specific protocols are possible without departing from the present invention.

### EXAMPLES

Combination of the TLR7/8 agonist R848 with 4SC-202 ((E)-N-(2-amino-phenyl)-3-{1-[8-(1-methyl-1H-pyrazol-4-yl)-benzenesulfonyl]-1H-pyrrol-3-yl}-acrylamide) results in better anti-tumoral effect in animal model

Methods to determine the activities of agonists of TLR7 and/or TLR8 are known in the art, e.g., from the above-mentioned patent EP 2386557 B1.

### Compounds Stimulating TRL7 or TLR8 can be Determined in the Reporter Gene Assay:

Reporter gene expression can be analyzed in a cell line stably transfected with human TLR7 and/or TLR8, respectively, and coexpressing an NFkB/AP-1 inducible reporter gene (e.g. SEAP, luciferase, ect.). If SEAP (secreted embryonic alkaline phosphatase) is used as reporter gene, stimulation with a TLR ligand leads to NFkB/AP-1 activation and upregulation and secretion of SEAP, which can be measured in the supernatant with QuantiBlue™ detection system (InvivoGen). The cell lines used were either HEK-Blue-hTLR7 (InvivoGen #hkb-htlr7) or HEK-Blue-hTLR8 (InvivoGen #hkb-htlr8). Cells were cultured following manufacturer's instructions.

### Assay Protocol:

Cell suspensions of each cell line were prepared with Nv=0.11×10⁶ cells/ml in medium (DMEM 4.5 g/l Glucose, 2-4 mM L-glutamine+10% FBS) without antibiotics (Nv=number of living cells). 180 μl of the cell suspensions per well of a 96 well flat bottom cell culture plates (coster, #3506) were added and the plates were incubated overnight at 37° C. and 5% CO₂.

Samples and controls were prepared as solutions in DMSO: R848, CL075 (TLR 7/8 agonist by InvivoGen) and Guardiquimod each to a final concentration of 10 μM, ODN2006 (InvivoGen) and ODN2216 (InvivoGen) each to a final concentration 2 μM; compounds of the invention are applied in dilution series to final concentrations of 30.0 μM, 10.0 μM, 3.0 μM, 1.0 μM, etc.

20 μl diluted compounds and controls (see above) were added to the wells and the plates were incubated for 20-24 h at 37° C. and 5% CO₂.

QuantiBlue™ (InvivoGen #10H19-MM) solution was prepared following the manufacturer's instructions, i.e. The powder of one pouch was dissolved in 100 ml MilliQ grade water, heated at 37° C. in a water bath for about 30 min, and the solution was filtered with a paper filter.

Subsequently, 100 μl prepared QuantiBlue™ solution was added to each well of a 96 well plate with flat bottom and 25 μl cell culture supernatant from the induced cells per well was added. The plates were incubated for 1 to 1.5 h at 37° C. (until an intense blue color had developed). Thereafter, the plates were read at 620 nm with a plate reader to determine the OD. The EC50 value was calculated from data points.

### Animal Model Methodology:

Combined substance
a) (E)-N-(2-amino-phenyl)-3-{1-[4-(1-methyl-1H-pyrazol-4-yl)-benzenesulfonyl]-1H-pyrrol-3-yl}-acrylamide (4SC-202)
b) R848 (Resiquimod),

### Test and Combined Substance Vehicles

Every 5 days of administration to mice (E)-N-(2-amino-phenyl)-3-{1-[4-(1-methyl-1H-pyrazol-4-yl)-benzenesulfonyl]-1H-pyrrol-3-yl}-acrylamide is suspended in 2% methocel solution at 2 mg/ml (active compound). Further, on every day of injection to mice R848 will be diluted in NaCl 0.9% (Aguettant, France) at 0.2 mg/ml.

### Treatment Doses

(E)-N-(2-amino-phenyl)-3-{1-[4-(1-methyl-1H-pyrazol-4-yl)-benzenesulfonyl]-1H-pyrrol-3-yl}-acrylamide is administered at 20 mg/kg/adm twice daily. The dose strength represents active pharmaceutical ingredient. For actual administered doses the salt factor will be taken into account. R848 is injected at 2 mg/kg.

### Routes of Administration

(E)-N-(2-amino-phenyl)-3-{1-[4-(1-methyl-1H-pyrazol-4-yl)-benzenesulfonyl]-1H-pyrrol-3-yl}-acrylamide is administered by oral gavage (per os, PO) via a gavage tube. R848 is injected intravenously (IV, bolus) into the caudal vein of mice. In all cases, the administration volume will be 10 mF/kg adjusted to the most recent individual body weight of mice.

### Cancer Cell Line and Culture Conditions

Cancer cell line “Colon 38” (C38)—frozen tumor fragments were obtained from the Division of Cancer Treatment, Tumor Repository, NCI (Frederick, Md., USA) provided by Oncodesign. The C38 cell line is a C57BL/6J mouse colon adenocarcinoma cell line.

### In Vivo Tumour Amplification

The C38 fragments are stored frozen in DMSO/SVF/RPMI 1640 medium (10/10/80) in liquid nitrogen until use. In study part 1, C38 frozen fragments are thawed at 37° C. for 5 min, rinsed twice in RPMI1640 medium before subcutaneous (SC) implantation in mice.

Female C57BL/6J mice are subcutaneously implanted into the right flank with C38 tumour fragments. When tumour volumes reach 500-1000 mm³, tumours are surgically excised and tumour fragments (30-50 mg) are subcutaneously implanted into the right flank of 80 female C57BL/6J mice at day 0 (DO).

### Animals

Healthy female C57BL/6J (C57Bl/6JRj) mice, of matching weight and age, obtained from Janvier (France) are used.

### Treatment Schedule

The treatment starts when the tumors reach a mean volume of 100-200 mm³ (day 10). Mice are randomized according to their individual tumor volume into 4 groups of each of 20 animals using Vivo Manager® software (Biosystemes, Couternon, France). A statistical test (analysis of variance, ANOVA) is performed to test for homogeneity between groups.

The treatment schedule is as follows:
Animals in group 1 receive twice daily PO administrations of vehicle for 24 consecutive days (2Q1D×24). The bi-daily treatments are separated by a 12 hour period,
Animals in group 2 receive twice daily PO administrations of 4SC-202 ((E)-N-(2-amino-phenyl)-3-{1-[4-(1-methyl-1H-pyrazol-4-yl)-benzenesulfonyl]-1H-pyrrol-3-yl}-acrylamide) at 20 mg/kg/adm for 24 consecutive days (2Q1D×24). The bi-daily treatments will be separated by a 12 hour period,
Animals in group 3 receive one IV injection of R848 at 2 mg/kg every 5 days three times (Q5D×3),
Animals in group 6 receive one IV injection of R848 at 2 mg/kg every 5 days three times (Q5D×3) in combination with twice daily PO administrations of 4SC-202 ((E)-N-(2-amino-phenyl)-3-{1-[4-(1-methyl-1H-pyrazol-4-yl)-benzenesulfonyl]-1H-pyrrol-3-yl}-acrylamide) at 20 mg/kg/adm for 24 consecutive days (2Q1D×24). The bi-daily treatments will be separated by a 12 hour period. The R848 treatment will be performed 6 hours after the 4SC-202 ((E)-N-(2-amino-phenyl)-3-{1-[4-(1-methyl-1H-pyrazol-4-yl)-benzenesulfonyl]-1H-pyrrol-3-yl}-acrylamide) morning treatment,

The treatment schedule is summarized in table 1 below:

TABLE 1

| | No | | Dose | Adm. | Treatment |
|---|---|---|---|---|---|
| Group | animals | Treatment | (mg/kg/adm or inj). | Route | schedule |
| 1 | 20 | Vehicle | — | PO | 2Q1Dx24* |
| 2 | 20 | 4SC-202 | 20 | PO | 2Q1Dx24* |
| 3 | 20 | R848 | 2 | IV | Q5Dx3** |
| 6 | 20 | 4SC-202 | 20 | PO | 2Q1Dx24* |
| | | R848 | 2 | IV | Q5Dx3** |

*The 4SC-202 bi-daily treatments will be separated by a 12 hour period.
**The R848 injections will be performed 6 hours after the morning 4SC202 PO administrations.

### Tumour Collection

Ten mice in each group are sacrificed at D28. The day of termination the mice treated with 4SC-202 will received only the morning treatment.

### Animal Monitoring

Clinical monitoring: All study data, including animal body weight measurements, tumor volume, clinical and mortality records, and treatment are scheduled and recorded on Vivo Manager® database (Biosystemes, Dijon, France). The viability and behavior are recorded every day. Body weights are measured twice a week. The length and width of the tumor are measured twice a week with calipers and the volume of the tumor will be estimated by the formula [4]:

$$Tumor volume = \frac{w i d t {h}^{2} \times length}{2}$$

### Results

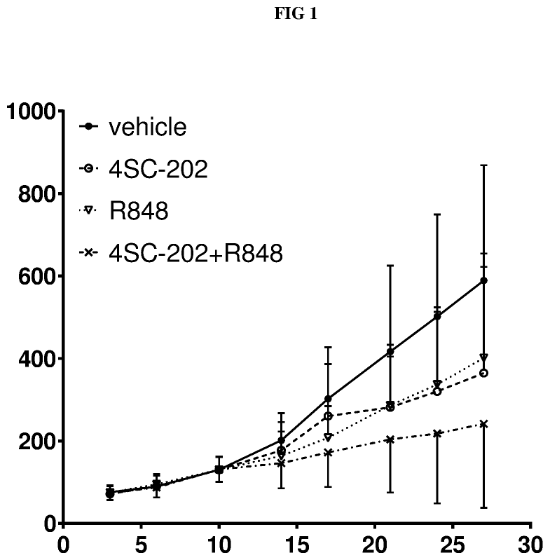

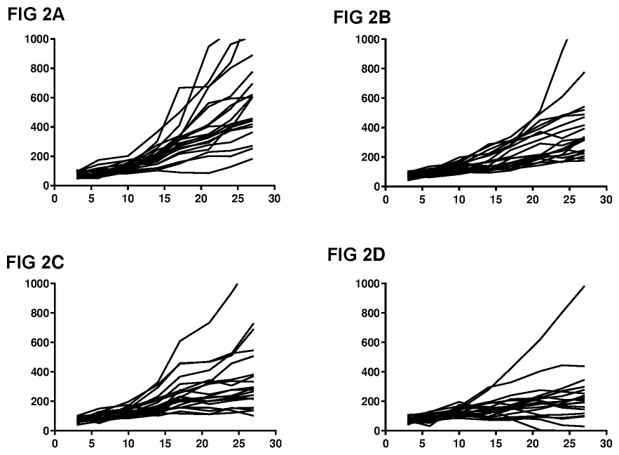

Unlike in animals treated with the inventive pharmaceutical combination, no tumor regression was observed in mono-therapies (FIG. 1). FIG. 2 shows that even complete remission was observed in some of the treated animals, however, only in the combination group. Data past day 27 not shown.

1. A pharmaceutical combination product comprising a compound of Formula (I) 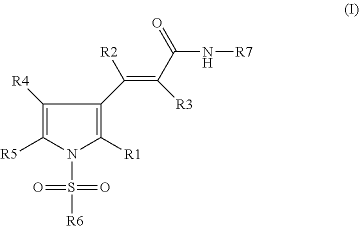
in which
R1, R4 and R5 are independently hydrogen, 1-4C-alkyl, halogen, or 1-4C-alkoxy,
R2 and R3 are independently hydrogen or 1-4C-alkyl,
R6 is -T1Q1, in which T1 is a bond or 1-4C-alkylene,
either Q1 is substituted by R61 and/or R62, and is Aa1, Hh1, Ha1, Ha2, Ha3, Ha4 or Ah1, or
Q1 is unsubstituted, and is Ha2, Ha3 or Ha4, in which
R61 is 1-4C-alkyl, phenyl-1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, hydroxy-1-4C-alkyl, 1-4C-alkoxy-1-4C-alkyl, 1-4C-alkylsulphonylamino, tolylsulphonylamino, phenylsulphonylamino, 1-4C-alkylcarbonylamino, carbamoyl, sulphamoyl, mono- or di-1-4C-alkylaminocarbonyl, mono- or di-1-4C-alkylaminosulphonyl, -T2-N(R611)R612, —U-T3-N(R613)R614, -T4-Het3, or —V-T5-Het4, in which
T2 is a bond or 1-4C-alkylene,
R611 is hydrogen, 1-4C-alkyl, 3-7C-cycloalkyl, 3-7C-cycloalkylmethyl, hydroxy-2-4C-alkyl, 1-4C-alkoxy-2-4C-alkyl, 1-4C-alkylcarbonyl, or 1-4C-alkylsulphonyl,
R612 is hydrogen or 1-4C-alkyl,
or R611 and R612 together and with inclusion of the nitrogen atom, to which they are bonded, form a heterocyclic ring Het1, in which Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl, 3-7C-cycloalkylmethyl, hydroxy-2-4C-alkyl or 1-4C-alkoxy-2-4C-alkyl, 1-4C-alkylcarbonyl, or 1-4C-alkylsulphonyl
R614 is hydrogen or 1-4C-alkyl,
or R613 and R614 together and with inclusion of the nitrogen atom, to which they are bonded, form a heterocyclic ring Het2, in which
Het2 is morpholino, thiomorpholino, S-oxo-thiomorpholino, S,S-dioxo-thiomorpholino, piperidino, pyrrolidino, piperazino, or 4N-(1-4C-alkyl)-piperazino,
T4 is a bond or 1-4C-alkylene,
Het3 is 1N-(1-4C-alkyl)-piperidinyl or 1N-(1-4C-alkyl)-pyrrolidinyl,
V is —O— (oxygen) or —C(O)NH—,
T5 is a bond or 1-4C-alkylene,
Het4 is 1N-(1-4C-alkyl)-piperidinyl or 1N-(1-4C-alkyl)-pyrrolidinyl,
R62 is 1-4C-alkyl, 1-4C-alkoxy or halogen,
Aa1 is a bisaryl radical made up of two aryl groups,
which are selected independently from a group consisting of phenyl and naphthyl, and
which are linked together via a single bond,
Hh1 is a bisheteroaryl radical made up of two heteroaryl groups,
which are selected independently from a group consisting of monocyclic 5- or 6-membered heteroaryl radicals comprising one or two heteroatoms, each of which is selected from the group consisting of nitrogen, oxygen and sulfur, and
which are linked together via a single bond,
Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
Ha4 is a heteroarylaryl radical made up of a heteroaryl group selected from a group consisting of partially saturated fused bicyclic 9- or 10-membered heteroaryl radicals comprising a heteroatom-free benzene ring and one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha4 is bonded via said aryl moiety to the to the parent molecular group,
R7 is hydroxyl, or Cycl, in which Cycl is a ring system of formula Ia 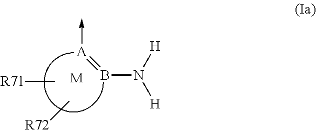
in which
A and B are C (carbon),
R71 and R72 are independently hydrogen, halogen, 1-4C-alkyl, or 1-4C-alkoxy,
M with inclusion of A and B is either a ring Ar2 or a ring Har2, in which Ar2 is a benzene ring,
Har2 is a monocyclic 5- or 6-membered unsaturated heteroaromatic ring comprising one to three heteroatoms, each of which is selected from the group consisting of nitrogen, oxygen and sulfur, and/or a salt or solvate of these compounds,
further comprising a TLR7 and/or TLR8 agonist for use in the treatment of cancer.

2. The pharmaceutical combination product comprising a compound of Formula (I) and a TLR7 and/or TLR8 agonist for use in the treatment of cancer according to claim 1,
wherein in the compound of formula (I),
R1, R4 and R5 are independently hydrogen, 1-4C-alkyl, halogen, or 1-4C-alkoxy,
R2 and R3 are independently hydrogen or 1-4C-alkyl,
R6 is -T1-Q1, in which T1 is a bond or 1-4C-alkylene,
either
Q1 is substituted by R61 and/or R62, and is Aa1, Hh1, Ha1, Ha2, Ha3 or Ah1,
or
Q1 is unsubstituted, and is Ha2 or Ha3,
in which
R61 is 1-4C-alkyl, phenyl-1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, hydroxy-1-4C-alkyl, 1-4C-alkoxy-1-4C-alkyl, 1-4C-alkylsulphonylamino, tolylsulphonylamino, phenylsulphonylamino, 1-4C-alkylcarbonylamino, carbamoyl, sulphamoyl, mono- or di-1-4C-alkylaminocarbonyl, mono- or di-1-4C-alkylaminosulphonyl, -T2-N(R611)R612, or —U-T3-N(R613)R614, in which
T2 is a bond or 1-4C-alkylene,
R611 is hydrogen, 1-4C-alkyl, 3-7C-cycloalkyl, 3-7C-cycloalkylmethyl, hydroxy-2-4C-alkyl or 1-4C-alkoxy-2-4C-alkyl,
R612 is hydrogen or 1-4C-alkyl,
or
R611 and R612 together and with inclusion of the nitrogen atom, to which they are bonded, form a heterocyclic ring Het1, in which
Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
U is —O— (oxygen) or —C(O)NH—,
T3 is 2-4C-alkylene,
R613 is hydrogen, 1-4C-alkyl, 3-7C-cycloalkyl, 3-7C-cycloalkylmethyl, hydroxy-2-4C-alkyl or 1-4C-alkoxy-2-4C-alkyl,
R614 is hydrogen or 1-4C-alkyl,
or R613 and R614 together and with inclusion of the nitrogen atom, to which they are bonded, form a heterocyclic ring Het2, in which Het2 is morpholino, thiomorpholino, S-oxo-thiomorpholino, S,S-dioxo-thiomorpholino, piperidino, pyrrolidino, piperazino, or 4N-(1-4C-alkyl)-piperazino,
R62 is 1-4C-alkyl, 1-4C-alkoxy or halogen,
Aa1 is a bisaryl radical made up of two aryl groups,
which are selected independently from a group consisting of phenyl and naphthyl, and which are linked together via a single bond,
Hh1 is a bisheteroaryl radical made up of two heteroaryl groups,
which are selected independently from a group consisting of monocyclic 5- or 6-membered heteroaryl radicals comprising one or two heteroatoms, each of which is selected from the group consisting of nitrogen, oxygen and sulfur, and
which are linked together via a single bond,
Ah1 is an arylheteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
R7 is hydroxyl, or Cycl, in which Cycl is a ring system of formula Ia 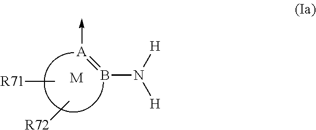
in which
A and B are C (carbon),
R71 and R72 are independently hydrogen, halogen, 1-4C-alkyl, or 1-4C-alkoxy,
M with inclusion of A and B is either a ring Ar2 or a ring Har2, in which
Ar2 is a benzene ring,
Har2 is a monocyclic 5- or 6-membered unsaturated heteroaromatic ring comprising one to three heteroatoms, each of which is selected from the group consisting of nitrogen, oxygen and sulfur,
and/or the salts or solvates or of these compounds
and
a TLR7 and/or TLR8 agonist.

3. The pharmaceutical combination product comprising a compound of Formula (I) and a TLR7 and/or TLR8 agonist for use in the treatment of cancer according to claim 1,
wherein in the compound of formula (I),
R1, R4 and R5 are independently hydrogen, 1-4C-alkyl, halogen, or 1-4C-alkoxy,
R2 and R3 are independently hydrogen or 1-4C-alkyl,
R6 is -T1-Q1, in which T1 is a bond, or 1-4C-alkylene,
either
Q1 is substituted by R61 and/or R62, and is Aa1, Hh1, Ha1, Ha2, Ha3 or Ah1,
or Q1 is unsubstituted, and is Ha2 or Ha3,
in which
R61 is 1-4C-alkyl, 1-4C-alkoxy, hydroxyl, trifluoromethyl, cyano, halogen, completely fluorine-substituted 1-4C-alkoxy or 1-4C-alkoxy wherein more than half of the hydrogen atoms are replaced by fluorine atoms, or -T2-N(R611)R612, in which
T2 is a bond or 1-4C-alkylene,
R611 and R612 are independently hydrogen or 1-4C-alkyl,
or
R611 and R612 together and with inclusion of the nitrogen atom, to which they are bonded, form a heterocyclic ring Het1, in which
Het1 is morpholino, thiomorpholino, S-oxo-thiomorpholino, S,S-dioxo-thiomorpholino, piperidino, pyrrolidino, piperazino, or 4N-(1-4C-alkyl)-piperazino,
R62 is 1-4C-alkyl, 1-4C-alkoxy or halogen,
Aa1 is a bisaryl radical made up of two aryl groups,
which are selected independently from a group consisting of phenyl and naphthyl, and which are linked together via a single bond,
Hh1 is a bisheteroaryl radical made up of two heteroaryl groups,
which are selected independently from a group consisting of monocyclic 5- or 6-membered heteroaryl radicals comprising one or two heteroatoms, each of which is selected from the group consisting of nitrogen, oxygen and sulfur, and
which are linked together via a single bond,
Ah1 is an aryl-heteroaryl radical made up of an aryl group selected from a group consisting of phenyl and naphthyl, and a heteroaryl group selected from a group consisting of monocyclic 5- or 6-membered heteroaryl radicals comprising one or two heteroatoms, each of which is selected from the group consisting of nitrogen, oxygen and sulfur, whereby said aryl and heteroaryl groups are linked together via a single bond, and whereby Ah1 is bonded via said heteroaryl moiety to the parent molecular group,
Ha1 is a heteroarylaryl radical made up of a heteroaryl group selected from a group consisting of monocyclic 5- or 6-membered heteroaryl radicals comprising one or two heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha1 is bonded via said aryl moiety to the to the parent molecular group,
Ha2 is a heteroarylaryl radical made up of a heteroaryl group selected from a group consisting of fused bicyclic 9- or 10-membered heteroaryl radicals comprising one, two or three heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha2 is bonded via said aryl moiety to the parent molecular group,
Ha3 is a heteroarylaryl radical made up of a heteroaryl group selected from a group consisting of monocyclic 5-membered heteroaryl radicals comprising three or four heteroatoms, each of which is selected from the group consisting of nitrogen, oxygen and sulfur, and an aryl group selected from a group consisting of phenyl and naphthyl, whereby said heteroaryl and aryl groups are linked together via a single bond, and whereby Ha3 is bonded via said aryl moiety to the to the parent molecular group,
R7 is hydroxyl, or Cycl, in which Cycl is a ring system of formula Ia 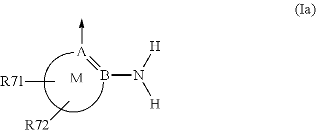
in which
A and B are C (carbon),
R71 and R72 are independently hydrogen, halogen, 1-4C-alkyl, or 1-4C-alkoxy,
M with inclusion of A and B is either a ring Ar2 or a ring Har2, in which Ar2 is a benzene ring,
Har2 is a monocyclic 5- or 6-membered unsaturated heteroaromatic ring comprising one to three heteroatoms, each of which is selected from the group consisting of nitrogen, oxygen and sulfur,
and/or a salt or a solvate of these compounds.

4. The pharmaceutical combination product comprising a compound of Formula (I) and a TLR7 and/or TLR8 agonist for use in the treatment of cancer according to claim 1, wherein in the compound of formula (I) is (E)-N-(2-amino-phenyl)-3-{1-[4-(1-methyl-1H-pyrazol-4-yl)-benzenesulfonyl]-1H-pyrrol-3-yl}-acrylamide or a salt or solvate thereof.

5. The pharmaceutical combination product comprising a compound of Formula (I) and a TLR7 and/or TLR8 agonist for use in the treatment of cancer according to claim 1, wherein in the compound of formula (I) is (E)-N-(2-amino-phenyl)-3-{l-[4-(1-methyl-1H-pyrazol-4-yl)-benzenesulfonyl]-1H-pyrrol-3-yl}-acrylamide or salt thereof, wherein the salt is the tosylate salt.

6. The pharmaceutical combination product comprising a compound of Formula (I) and a TLR7 and/or TLR8 agonist for use in the treatment of cancer according to claim 1, wherein the TLR7 and/or TLR8 agonist is selected from the group comprising Gardiquimod, Imiquimod and R848 (resiquimod).

7. The pharmaceutical combination product comprising a compound of Formula (I) and a TLR7 and/or TLR8 agonist for use in the treatment of cancer according to claim 1, wherein the TLR7 and/or TLR8 agonist is resiquimod.

8. The pharmaceutical combination product comprising a compound of Formula (I) and a TLR7 and/or TLR8 agonist for use in the treatment of cancer according to claim 1, wherein the compound of formula (I) and the TLR7 and/or TLR8 agonist are administered concomitantly or separately.

9. The pharmaceutical combination product comprising a compound of Formula (I) and a TLR7 and/or TLR8 agonist for use in the treatment of cancer according to claim 1, wherein the compound of formula (I) and the TLR7 and/or TLR8 agonist are administered separately.

10. The pharmaceutical combination product comprising a compound of Formula (I) and a TLR7 and/or TLR8 agonist for use in the treatment of cancer according to claim 1, wherein the compound of Formula (I) is administered initially and the TLR7 and/or TLR8 agonist is administered subsequently.

11. The pharmaceutical combination product comprising a compound of Formula (I) and a TLR7 and/or TLR8 agonist for use in the treatment of cancer according to claim 1, wherein the compound of Formula (I) is formulated for oral administration and the TLR7 and/or TLR8 agonist is formulated for parenteral or enteral administration.

12. The pharmaceutical combination product comprising a compound of Formula (I) and a TLR7 and/or TLR8 agonist for use in the treatment of cancer according to claim 1, wherein the cancer is selected from the group comprising hepatocarcinoma, adrenocortical carcinoma, AIDS-related cancers including AIDS-related lymphoma, anal cancer, basal cell carcinoma, bile duct cancer, bone cancer, brain tumors including brain stem glioma, cerebellar astrocytoma, cerebral astrocytoma, malignant glioma, ependymoma, medulloblastoma, supratentorial primitive neuroectodermal tumors, visual pathway and hypothalamic glioma, breast cancer, bronchial adenomas/carcinoids, Burkitt's lymphoma, gastrointestinal, carcinoma of unknown primary site, central nervous system lymphoma, cervical cancer, chronic myeloproliferative disorders, colon cancer, colorectal cancer, cutaneous T-cell lymphoma, endometrial cancer, ependymoma, esophageal cancer, extracranial germ cell tumor, extragonadal germ cell tumor, ovarian germ cell tumor, eye cancer including intraocular melanoma and retinoblastoma, gallbladder cancer, gastrointestinal carcinoid tumor, gestational trophoblastic tumor, glioma, childhood brain stem glioma, head and neck cancer, hematologic cancer, adult and childhood (primary) hepatocellular cancer, hypopharyngeal cancer, islet cell or pancreatic cancer, renal cancer, laryngeal cancer, acute lymphoblastic leukemia, adult and childhood acute myeloid leukemia, chronic lymphocytic leukemia, chronic myelogenous leukemia, hairy cell leukemia, lip and oral cavity cancer, liver cancer, lung cancer, including non-small cell lung cancer and small cell lung cancer, Hodgkin's lymphoma, non-Hodgkin's lymphoma, primary central nervous system lymphoma, Waldenstrom's macroglobulinemia, merkel cell carcinoma, mesothelioma, metastatic squamous neck cancer with occult primary site, multiple endocrine neoplasia syndrome, multiple myeloma/plasma cell neoplasm, mycosis fungoides, myelodysplastic syndromes, myelodysplastic myeloproliferative diseases, multiple myeloma, chronic myeloproliferative disorders, nasal cavity and paranasal sinus cancer, nasopharyngeal cancer, neuroblastoma, oral cancer, oropharyngeal cancer, osteosarcoma/malignant fibrous histiocytoma of bone, ovarian cancer, ovarian epithelial cancer, ovarian low malignant potential tumor, pancreatic cancer, parathyroid cancer, penile cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter cancer, transitional cell cancer, rhabdomyosarcoma, salivary gland cancer, Ewing's sarcoma, Kaposi's sarcoma, soft tissue sarcoma, uterine sarcoma, sezary syndrome, skin cancer, including melanoma and non-melanoma skin cancer, small intestine cancer, squamous cell carcinoma, gastric cancer, supratentorial primitive neuroectodermal tumors, testicular cancer, thymoma, thymoma and thymic carcinoma, thyroid cancer, trophoblastic tumor, gestational, endometrial uterine cancer, uterine sarcoma, vaginal cancer, vulvar cancer, Waldenstrom's macroglobulinemia, Wilms' tumor.

13. The pharmaceutical combination product comprising a compound of Formula (I) and a TLR7 and/or TLR8 agonist for use in the treatment of cancer according to claim 1, wherein the cancer is selected from the group comprising cancer of the prostate, bladder, kidney, muscle, ovary, skin, lung, pancreas, breast, cervix, colon, liver, connective tissue, placenta, bone, brain, uterus, salivary gland, or testes.
